(19) United States
(12) Patent Application Publication
(10) Pub. No.: US 2013/0266543 A1
(43) Pub. Date: Oct. 10, 2013

(54) Multipotent Adult Stem Cell Population

(71) Applicant: Bernardo Nadal-Ginard, Madrid (ES)

(72) Inventor: Bernardo Nadal-Ginard, Madrid (ES)

(21) Appl. No.: 13/801,213

(22) Filed: Mar. 13, 2013

Related U.S. Application Data
(62) Division of application No. 13/446,466, filed on Apr. 13, 2012.
Application No. 13/446,466 is a continuation of application No. 13/367,069, filed on Feb. 6, 2012, now abandoned.
Application No. 13/367,069 is a continuation of application No. 13/163,587, filed on Jun. 17, 2011, now abandoned.
Application No. 13/163,587 is a continuation of application No. 12/886,538, filed on Sep. 20, 2010, now abandoned.
Application No. 12/886,538 is a continuation of application No. 12/657,710, filed on Jan. 25, 2010, now abandoned.
Application No. 12/657,710 is a continuation of application No. 12/387,862, filed on May 8, 2009, now abandoned.
Application No. 12/387,862 is a continuation of application No. PCT/IB2009/005636, filed on May 8, 2009.
(60) Provisional application No. 61/127,067, filed on May 8, 2008.

(51) Int. Cl. A61K 35/34

(52) U.S. Cl. CPC A61K 35/34
USPC 424/93.7; 435/325; 435/34; 435/7.1

(57) ABSTRACT

The present invention relates to the identification, isolation, expansion and characterization of a specific type of adult stem cell. These adult stem cells are characterized in that they naturally express many of the markers of totipotency, which have hitherto generally been limited to embryonic cell populations. The cells of the invention display an unprecedented capacity for multi potency; they are able to differentiate into cell types of mesodermal, endodermal and ectodermal origin. These adult stem cells may be used as therapeutic agents including, without limitation, for the regeneration of tissue, particularly for regeneration of damaged cardiac tissue, such as myocardium.

### FIELD OF THE INVENTION

The present invention relates to the identification, isolation, expansion and characterization of a specific type of adult stem cell. These adult stem cells are characterised in that they naturally express many of the markers of totipotency, which have hitherto generally been limited to embryonic cell populations. The cells of the invention display an unprecedented capacity for multipotency; they are able to differentiate into cell types of mesodermal, endodermal and ectodermal origin. These adult stem cells may be used as therapeutic agents including, without limitation, for the regeneration of tissue, particularly for regeneration of damaged cardiac tissue, such as myocardium.

### BACKGROUND

Totipotent stem cells have a specific phenotype, and, amongst other things, are capable of differentiating into any type of cell, including those derived from endoderm, ectoderm and mesoderm. Classically, embryonic stem cells were thought to be the only type of cells which exhibited this functionality. Embryonic stem cells must be isolated from embryos, and for this reason, their use in therapy raises a number of ethical considerations. In addition, embryonic stem cells are not genetically identical to adult hosts in need of stem cell based therapeutics, which could lead to rejection of the stem cells by the adult host, particularly after differentiation into cell types that express MHC I or MHC II.

More recently, adult stem cells have been described which apparently have the capacity to differentiate into multiple different tissues (Beltrami et al., 2003. Cell 114: 763-776). These adult stem cells were originally perceived to represent adult remnants of embryonic stem cells. However, these adult stem cells in fact show a different phenotype and a more restricted development potential from that of embryonic stein cells. It appears as if these cells may be descendants of a more primitive adult stem cell, and so may already be partially committed to a specific differentiation fate. As such, they may only be capable of differentiating into the various cell types of the organ from which they originated.

From a therapeutic perspective, it has long been established that embryonic stem cells have the capacity to facilitate tissue regeneration upon injection into a damaged tissue. However, as mentioned above, this causes a variety of ethical problems, not least due to the method by which these cells must be collected. Furthermore, injection of cells of this type into adult animals generates teratomas in a large percentage of cases, so posing a very significant limitation to their use in a therapeutic context. Finally, as discussed above, such stem cells are not genetically identical to the adult host and therefore may be rejected by the host.

This latter drawback appeared to have been solved by the newly described “induced stem cells” (iES cells) (Takahashi et al., 2007. Cell 131: 1-12), whereby transformation of a variety of somatic stem cells with 4 genes commonly expressed in bona fide embryonic stem cells caused the somatic cells to acquire the multipotent characteristics of an embryonic stem cell, whilst retaining the genetic identity of the somatic cell. Unfortunately, it is now clear that these adult-derived multipotent cells also display a tendency to form teratomas upon injection into adult animals, and their use as a therapeutic is therefore limited.

There therefore remains an on-going requirement to identify a population of stem cells which have the potential to differentiate into mesoderm, endoderm and ectoderm derived parenchymal cells, but which do not have a propensity to form teratomas upon injection into patients. Further, there is a need for such cells that may be isolated from an adult so as to be genetically identical to the adult and therefore not at risk of immune rejection.

### SUMMARY OF THE PREFERRED EMBODIMENTS

An aspect of the present invention relates to the discovery of a population of non-germ adult stem cells that can be found in tissue from non-embryonic mammals, including at least mouse, rat, pig and human. These adult stem cells, which are present within the post-natal individual at all ages from infancy to senescence have a phenotype and developmental potential that is different from stem cell types, embryonic and adult, that have so far been described.

These adult stem cells have a remarkable multipotent capacity. As defined below, the cells are therefore capable of differentiating into the cells types derived from the three embryonic cell layers: mesoderm, endoderm and ectoderm.

These cells naturally express at least one of a variety of biochemical markers at the mRNA and protein level, some of which have been previously associated with a capacity for totipotency. Interestingly, these cells naturally express four transcription factor-encoding genes which have recently been shown to be able to confer totipotency when transfected into adult somatic fibroblasts: oct4, sox2, c-myc and Klf-4 (Takahashi et al, Cell 2007, 131 pg. 1-12). The cells also express Nanog, which is one of the best studied genes contributing to the multipotent state. Thus the cells of the invention, despite being present in post-natal and adult tissue, possess many of the biochemical and developmental properties that are classically associated with embryonic stem cells.

The adult stem cell population which is one embodiment of the invention is characterised by natural expression of one or more of the markers c-kit, Nanog and Oct-4 at a significant level. By “natural expression” is meant that the cells have not been manipulated recombinantly in any way, i.e., the cells have not been artificially induced to express these markers or to modulate these markers' expression by introduction of exogenous genetic material, such as introduction of heterologous (non-natural) or stronger promoters or other regulatory sequences operably linked to either the endogenous genes or exogenously-introduced forms of the genes. Natural expression is from genomic DNA within the cells, including introns between the exon coding sequences where these exist. Natural expression is not from cDNA. Natural expression can if necessary be proven by any one of various methods, such as sequencing out from within the reading frame of the gene to check that no extraneous heterogenous sequence is present.

The cells may also naturally express one or more of Rex1, Mph1, Eed and Mlc2a. The cells may also naturally express one or more of MDR-1, TERT, CD133, Gata-4, Gata-6, SOX-2, klf-4, c-myc, CD90, CD166, SSEA-1, and Bmi-1. The cells may also naturally express one or more of Isl-1, FoxD3, Mel-18, M33, Mph1/Rae-28, SDF1/CXCL12, BMP2, BPM-4, Wnt-3A, Wnt-4, and Wnt-11.

The adult stem cell population can also be characterised by a lack of natural expression of certain markers at any significant level, many of which are associated with cellular differentiation. Specifically, the cells of the isolated adult stem cell population do not naturally express one or more of Cd11b, CD13, CD14, CD29, CD31, CD33, CD36, CD38, CD49f, CD62, CD73, CD105, and CD106 at a significant level.

Another characteristic feature of the cells of the invention is that they do not elicit an immune response when brought into contact with cells of the immune system of an unmatched host or when injected into an unmatched allogenic recipient. One reason for this low level immunogenicity is that the cells of the invention do not express or express low levels of either MHC I in the case of the mouse or HLA major antigens in the case of the human. In addition, these cells may not express detectable levels or very low levels of co-adjuvant genes. Included within the definition of MHCI co-adjuvant genes are CD86, CD80, CD40, tapasin, TAP, calreticulin, calnexin and Erp57. This list is included by way of illustration only, and is not intended to be limiting. Another reason for the low immunogenicity is that the cells of the invention do not express MHC II, or that they express MHC II at a very low level. Further, even if the cells differentiate into cell types that express MHC I or MHC II, the adult stem cell, unlike embryonic stem cells, may be isolated from the subject intended to receive such stem cells so that the cells are genetically identical to the subject, and the potential problem of rejection does not arise.

Significant functional differences also exist between embryonic stem cells and the cells of the invention. It is well known in the art that embryonic stem cells, when transplanted into immunodeficient or syngeneic animals, have a very high propensity to generate teratomas in a dose-dependent manner. However, the cells of the invention do not exhibit any such propensity, and their potential use as therapeutics is therefore greatly enhanced.

The cells of the invention can be isolated from any one of a number of tissues, including, for example, cardiac tissue, brain, skeletal muscle, ovary, testicle and bone marrow. This list is provided by way of example only, and is not intended to be limiting.

When taken from cardiac tissue, such cells may for example be isolated from cardiac biopsies obtained during cardiac surgery, by means of a biopsy catheter during cardiac catheterism, or from the hearts of sacrificed animals. When isolated from cardiac tissue, the cells of the invention can easily differentiate into the three main cardiac cell lineages: cardiomyocytes, and smooth and endothelial cells of the microvasculature. Such differentiation has been shown by the inventors to occur both in vivo and in vitro. Upon differentiation, the cells secrete a large battery of growth factors and cytokines which are able to protect the myocardium from ischemic damage, inhibit the inflammatory response which occurs following myocardial death, and activate the growth and differentiation of the resident cardiac stem cells, their progenitors and precursors which contribute to the regeneration of the damaged contractile cells and microvasculature.

The progeny of a single clonal cell can be expanded through hundreds of passages for several years without the appearance of detectable chromosomal abnormalities, or the loss of the growth and differentiation properties of the cells.

When grown in suitable culture media, the cells of the invention can be induced to differentiate into a large variety of cell types, such as cardiac muscle cells, skeletal muscle cells, neurons, glia, smooth muscle, endothelial cells, skeletal muscle, bone, adipose tissue, etc., among other examples which will be clear to those of skill in the art. Cells of the invention are tripotent, and therefore have the capacity to differentiate into cells typical of each of the three embryonic cell layers; endoderm, ectoderm and endoderm, and thus to differentiate into any of the cells in the body.

The cells isolated from heart, brain, bone marrow and skeletal muscle show a tendency to spontaneously differentiate in vitro into cell types of their tissue of origin, such as neurons and glia, cells of the red and white cell blood lineage, and skeletal myocytes. However, when grown in culture media specific for other cell types, they readily differentiate into these other types. The frequency of “trans-differentiation” of the cell originated from different tissue types is similar.

In one embodiment, cells of the invention may differentiate into neural cells when cultivated as embryoid bodies (EBs) by the hanging drop method in differentiation medium I (DMI) or differentiation medium II (DMII) supplemented with specific differentiation factors. DMI contains 20% FCS, 2 mM L-glutamine, 1× MEM non-essential aminoacids, 0.1 mM β-ME, 50 μg/ml gentamycin, 0.1 mg/ml streptomycin, 100 U/ml penicillin (Sigma), 250 ng/ml amphotericin B, and 205 ng/ml sodium deoxycholate (Fungizone, Invitrogen) in Dulbecco's medium (DMEM, Gibco). DMII contains 15% DCC-treated FBS, 1× ITS supplement (Invitrogen), 2 mM L-glutamine, 1×MEM non-essential aminoacids, 0.1 mM β-ME, 50 μg/ml gentamycin, 0.1 mg/ml streptomycin, 100 U/ml penicillin (Sigma), 250 ng/ml amphotericin B, and 205 ng/ml sodium deoxycholate (Fungizone, Invitrogen) in Dulbecco's medium (DMEM, Gibco),

In one embodiment, 20 μl drops of differentiation medium containing cardiac Oct4^{pos} cells (n=80) may be placed on the lids of bacteriological Petri dishes filled with PBS containing 50 μg/ml gentamycin, 0.1 mg/ml streptomycin, 100 U/ml penicillin (Sigma), 250 ng/ml amphotericin B, 205 ng/ml sodium deoxycholate (Fungizone, Invitrogen) and cultured in hanging drops for up to 3 days. In one aspect of this embodiment, the cells may be cultured in hanging drops for 1 day, 2 days or 3 days. The cells may subsequently be cultured in bacteriological petri dishes for up to 4 days; for example, the cells may be cultured in bacteriological petri dishes for 1 day, 2 days, 3 days or 4 days. Following culture in hanging drops and bacteriological petri dishes the cells may be transferred to gelatine-coated dishes.

In one aspect, neural differentiation may be induced by culturing the cells of the invention in DMI or DMII supplemented with 100 ng/ml FGFb, 20 ng/ml EGF (Peprotech) and 1× B27 supplement with vitamin A (Invitrogen).

In one aspect, endothelial differentiation may be induced by culturing the cells of the invention in DMI or DMII supplemented with 10⁻⁸ dexamethasone (Sigma) and 10 ng/ml vascular endothelial growth factor (VEGF, Peprotech).

In one aspect, smooth muscle differentiation may be induced by culturing the cells of the invention in DMI or DMII supplemented with 50 ng/ml platelet-derived growth factor-BB (PDGF-BB, Peprotech).

In one aspect, cardiomyogenic lineage differentiation may be induced by culturing the cells of the invention in DMI or DMII supplemented with one or more of 1% dimethyl sulfoxide (DMSO), 10 μM 5-azacytidine, 10 μM oxytocin, 10⁻⁸ M retinoic acid, 0.1 mM ascorbic acid (Sigma), 29 nM FGFb, 2.5 ng/ml transforming growth factor beta-1 (TGFβ1), 4 nM cardiotrophin-1 (Peprotech), and 40 nM thrombin (Sigma).

In one aspect, differentiation of the cells of the invention into spontaneously beating cardiac cells may be induced by culturing the cells of the invention with 100 nM Oxytocin for 72 hours to generate cardiospheres prior to transferring the cells to laminin-coated plastic dishes with a myo-cardiogenic medium consisting of α-MEM (base medium), supplemented with 2% FBS, dexamethasone (1 μM), ascorbic acid (50 μg/ml), β-glycerophosphate (10 mM), TGF-β1 (5 ng/ml), BMP2 (10 ng/ml), and BMP4 (10 ng/ml) (FIG. 27B). After 4 days, TGF-β1, BMP2, and BMP4 may be removed from the media. For the remaining 10 days, the media may be supplemented with the canonical Wnt inhibitor, Dickkopf-1 (DKK-1; 150 ng/ml).

Included within the scope of the invention are methods of treatment of a human or animal patient through cellular therapy. Such cellular therapy encompasses the application of the stem cells of the invention to the patient through any appropriate means. Specifically, such methods of treatment involve the regeneration of damaged tissue. In accordance with the invention, a patient can be treated with allogeneic or autologous adult stem cells. “Autologous” cells are cells which originated from the same organism into which they are being re-introduced for cellular therapy, for example in order to permit tissue regeneration. However, the cells have not necessarily been isolated from the same tissue as the tissue they are being introduced into. An autologous cell does not require matching to the patient in order to overcome the problems of rejection. “Allogeneic” cells are cells which originated from an individual which is different from the individual into which the cells are being introduced for cellular therapy, for example in order to permit tissue regeneration, although of the same species. Some degree of patient matching may still be required to prevent the problems of rejection.

### BRIEF DESCRIPTION OF FIGURES

FIG. 1. Cellular and molecular characterization of stem cells from the adult murine heart

a; Q-PCR analysis in adult cardiac cells enriched and sorted using immunomagnetic beads for SSEA-1, c-kit and Sea-1, comparing mRNA levels of several genes involved in ESCs multipotency and maintenance of an undifferentiated state. b; Flow cytometry diagrams showing the purification of a c-kit^{pos} population from the small cells fraction of the adult murine myocardium. c; Illustrative example of the high purity (95%) of a sample sorted for c-kit. d; Flow cytometry graph showing c-kit expression and faint intrinsic Oct4-EGFP fluorescence in a representative sample of cardiac small cells.

FIG. 2. Oct4-EGFP^{pos} cells are present throughout the adult myocardium

a; Double immunostaining of freshly isolated adult murine cardiac cells expressing Oct4 and c-kit. b-k; Immunostaining for EGFP in cryosections showing presence of Oct4-EGFP^{pos} cells in different regions of the adult mouse heart. b, c; Higher density of Oct4-EGFP^{pos} cells in the region surrounding the outflow tract (Oft), at two magnifications. d-f; Examples of Oct4-EGFP^{pos} cells (*) in the ventricle, at two magnifications. g, h; An Oct4-EGFP^{pos} cell (*) in the septum, at two magnifications. i, j; Illustrative example of Oct4-EGFP^{pos} cell (*) in atrial epicardium and endocardium, at different magnifications. k, Confocal microscope z-axis projection of a representative Oct4-EGFP^{pos} cell. a, c-k; Counterstaining of nuclei with DAPI. Scale bars are 10 μm (a, g, h, k) and 50 μm (b-f, I, j).

FIG. 3. Spatial and age-related distribution of Oct4 EGFP^{pos} cells in the murine myocardium

a; Immunohistochemistry for EGFP showing representative examples of Oct4-EGFP^{pos} cells in different regions of the myocardium of newly born, 2 week old, 2 months old and 24 months old mice. The scale bar represents 10 μm. b; Histogram showing the abundance of Oct4-EGFP^{pos} cells in different regions of the murine heart with age.

FIG. 4. Molecular characterization of Oct4 cells from the adult murine heart

a; RT-PCR analysis of freshly isolated murine cardiac c-kit^{pos} cells. b; RT-PCR analysis of a representative Oct4-EGFP^{pos} long-term expanded adult mouse cardiac clone. c; Q-PCR comparative analysis of mRNA expression levels in freshly isolated cardiac c-kit^{pos} cells, a representative Oct4-EGFPpos long-term expanded adult mouse cardiac clone and ES-D3 mouse embryonic stem cells. M, 100 bp DNA markers. b, c; Different sets of primers (*, **) have been used to confirm the results. GAPDH mRNA was amplified as an internal control.

FIG. 5. In vitro differentiation of a mouse adult cardiac Oct4 clone

a, b; Examples of embryoid bodies formed with an Oct4 mouse cardiac clone by the “hanging drop” method (a) and after plating on a poly-L-Lysine-coated slide (b). c-e; Immunofluorescence for GFP in a representative embryoid body (c, EB) and in the cells that migrated attached to the slide (d, e). f-l, Immunofluorescence for neurofilament-H in the embryoid bodies (f-h, EB) and the migrating cells (i-l). a, b; Transmitted light. c-l; Fluorescent light. d-h, l, Counterstaining of nuclei with DAPI. Scale bars are 500 μm (a, b) and 50 μm (c-l). m; RT-PCR analysis showing in vitro differentiation of a cardiac Oct-4^{pos} lone into the neural, cardiomyogenic, smooth muscle and vascular endothelial lineages after culturing the embryoid bodies with specific differentiation media; U, undifferentiated, M, 100 bp DNA markers; GAPDH mRNA was amplified as an internal control.

FIG. 6. Cardiac Oct4 cells generate chimerism in chicken and mouse embryos

a: PCR analysis of genomic DNA obtained from the body and head regions of chicken embryos five days after injection into the amniotic cavity of 1×10⁵/1×10⁶ adult mouse cardiac cells derived from a single LacZ^{pos}/Oct4-EGFP^{pos} clone expanded in vitro. b; PCR analysis of genomic DNA obtained from 13.5 dpc mouse embryos (n=20) 10 days after injection into the blastocyst of 10-15 adult cardiac c-kit^{pos} cells freshly isolated from LacZ/Oct4-EGFP mice. a, b; GAPDH mRNA was amplified as an internal control. c; Flow cytometry analysis of the c-kit^{pos} sorted population used for injection into 3.5 dpc wild type blastocysts. d, e; Illustrative pictures of the least chimeric (d) and the most chimeric (e) 13.5 dpc embryos after X-gal staining. f-I, X-gal staining and immunohistochemistry for β-galactosidase showing the specific staining of cells derived from the injected cells in the intestine (In), peritoneum (Pe) and liver (Li) of chimeric embryos.

FIG. 7. In vitro differentiation of rat tripotent adult stem cells isolated from the rat heart

A, pseudo-embryoid bodies in suspension; E, one embryoid body attached initiating differentiation; B, c-kit staining of the CSCs; C and D, like ES cells the CSCs also secrete large amounts of nestin; F, cell differentiated into striated cardiac myocytes; G, cells differentiated into vascular smooth muscle cells; H, cell differentiated into capillaries.

FIG. 8. Chimerism in adult mice injected with an adult murine cardiac Oct4 clone

a-z; Chimerism screening in 1-3 month-old mice (n=71) derived from blastocysts which were injected at 3.5 dpc with 10-15 adult mouse cardiac cells obtained after expansion of a single LacZ^{pos}/Oct4-EGFP^{pos} clone. a; PCR analysis of genomic DNA isolated from tail biopsies of the injected mice one week after birth. b; PCR analysis of genomic DNA extracted from different tissues of the injected mice 1-3 months after birth. a, b; GAPDH was amplified as an internal control. M, 100 bp DNA markers. c-h, Spleen. i-k; bone marrow, l-n; lung. o, p; skin. q; intestine. r; heart. s-v; liver. w; brain. x-z; skeletal muscle. c, d, f-I, k-r, t-y; X-gal and immunohistochemistry for β-galactosidase; transmitted might. e, j, s, z; X-gal (black dots) and immunofluorescence for desmin and DAPI counterstaining of nuclei; fluorescent light. Scale bars are 50 μm (c, h, r, v) and 10 μm (d-g, i-q, s-u, w-z).

FIG. 9. Normal male caryotype of a Oct-4^{pos} porcine CSC

The cell was initially isolated from a juvenile male in June of 2005 and subsequently subjected to several rounds of cloning and sub-cloning.

FIG. 10. Human Oct 4^{pos} cells grow more rapidly than c-kit^{pos} cells. Left panel: clone 1 week after plating. Right panel: same clone 10 days after plating.

FIG. 11. Growth of human left ventricle explants for the isolation of Oct 4^{pos} cells.

FIG. 12. Characterization and isolation of cardiac mouse “side population” of cells.

Fluorometric histograms of cells from the heart (upper left panel) in which the side population is shown in green. This population disappears when the cells are treated with verapamil (upper right panel) which confirms their identity. The behaviour of the cardiac cells is identical to that of the bone marrow cells, as shown in the bottom left panel in which mononuclear bone marrow cells have been sorted with the same parameter used for the cardiac cells. The bottom right panel shows the phenotype of the cardiac “side population”. Only the Sca 1^{neg} c-kit^{pos} correspond to the Oct 4^{pos} cells of this invention

FIG. 13. PCR analysis of human Oct-4^{pos} CSCs isolated from the left ventricle.

FIG. 14. Human Oct-4^{pos} clone of cells induced to differentiate after forming pseudo-embryoid bodies.

Left panel: A, attached embryoid body a few days after plating; B, same body a few days later; C, DAPI staining to identify all nuclei; D, c-kit immunostaining. Note that many of the cells at the periphery are already c-kit^{neg} and Oct-4^{neg}. E, immunostaining for Oct-4. Note that only the cells at the centre of the clone remain fully undifferentiated and Oct-4^{pos}. Right panel: Same clone two weeks later. Upper panel: differentiated cardiac myocytes identified by the sarcomeric cardiac myosin; middle panel: smooth muscle vascular cells identified by immunostaining against smooth muscle myosin; bottom panel: endothelial cells identified by staining against von Villebrand factor. Nuclei were stained with DAPI.

FIG. 15. Human CSCs do not express either MHC-I locus or co-activator molecules.

This is in contrast with the robust expression of MHC-I in mesenchymal stem cells and MHC-I and CD-40 in adult fibroblasts.

FIG. 16. Myocardial sections of an infarcted ventricle from an immuno-deficient nu/nu rat, 10 days after the infarct and the inoculation of 1×10⁵ Oct-4^{pos} cells.

The human cells have differentiated into cardiac myocytes (left panel) stained with antibodies against cardiac myosin, arteriolar smooth muscle (middle panel), stained with anti-vascular smooth muscle myosin, and endothelial cell (left panel), stained with anti von Villebrand factor. The human cells can be identified by the punctate pattern of their nuclei produced by the hybridization of Alu-family human-specific DNA sequences. The rat nuclei (larger) are negative for the Alu sequences.

FIG. 17. Identification of the c-kit^{pos} Oct4^{pos} cells in tissue sections.

Section of rat ventricular myocardium stained with DAPI (lower left panel) to identify all the nuclei in the field. The four c-kit^{pos} in a cluster were labelled with an anti-c-kit monoclonal antibody (in red in the upper left panel) while the single c-kit^{pos} Oct4^{pos} in the cluster is identified in green in the upper right panel. The lower right panel show the merged image of the other three panels, which documents that there is a single c-kit^{pos} Oct4^{pos} cell in the image.

FIG. 18. Schematic representation of the sequence of development/differentiation of a single myocardial Oct4^{pos} cell.

The most primitive cell in this schematic representation is the c-kit^{pos} Oct4^{pos} which gives origin to the three main myocardial cell types: myocytes, endothelial and smooth muscle vascular cells. Upon differentiation the c-kit^{pos} Oct4^{pos} cell downregulates the expression of the multipotency genes and become c-kit^{pos} Oct4^{neg}. It is this cell population which generates three different cell lineages by turning on the expression of cell specific transcription factors. In a mutually exclusive manner each cell lineage gives origin to one of the three main myocardial cell lineages: endothelial, vascular smooth muscle and cardiomyocytes, as shown in the figure.

FIG. 19. In vitro functionally, anatomically and biochemically differentiated c-kit^{pos} Oct4^{pos} cells

Cloned c-kit^{pos} Oct4^{pos} cells grown in “differentiation medium” develop a fully differentiated phenotype as shown by bi-nucleation (in blue, stained with DAPI), fully developed sarcomeres shown with an antibody specific for sarcomeric α-actinin (in green) and with well formed gap junctions, as identified with an antibody specific for Connexin 43 (in red). This cell prior to fixation and staining was spontaneously beating.

FIG. 20. Self-renewal capability and cloning efficiency of the c-kit^{pos} Oct4^{pos} cells from different species.

Cells c-kit^{pos} Oct4^{pos} isolated from mouse, rat and different regions of the human heart were tested for their self-renewal capability by means of single cell cloning. Independently of the species or region of the myocardium from which the cells originated, all cell isolated showed a very high cloning efficiency for primary cells, which is evidence of their self-renewal capability.

FIG. 21. Isolation of c-kit^{pos} Oct4^{pos} cells by culturing cardiac small cells in “growth medium” supplemented with only 1% FCS.

The image shows an spontaneously formed pseudo-embryoid body of mouse cardiac cells transgenic for a construct driving GFP under the control of the Oct4 promoter. After two weeks in culture, many clones like the one shown were evident in the plate. Only the cells of these clones are GFP positive, while all the single cells remaining in the culture are GFP negative.

FIG. 22. Tropism of the c-kit^{pos} Oct4^{pos} cells for the tissue of origin when introduced into the systemic circulation. c-kit^{pos} Oct4^{pos} cells transgenic for GFP (panel A) were injected into the tail vein of rats after producing myocardial damage. As shown in panel B, large clusters of GFP positive cells can be identified in the ventricular myocardium at 7 days post-injection. These cells differentiate into integrated and striated myocytes at 2 weeks as shown in panel C.

FIG. 23. Expression of the major multipotency genes, Tert and NRx2.5 in freshly isolated murine c-kit^{pos} Oct4^{pos} cells.

The panels of the image show different fields of freshly isolated rat c-kit^{pos} Oct4^{pos} cells stained with antibodies specific for Nanog, Sox-2, Tert and NRx2.5. All the nuclei in the field are stained blue with Dapi while the proteins of interest are in green. It can be observed that most of the cells in each image are positive for the protein tested.

FIG. 24. Isolation, cloning and expansion of c-kit^{pos} Oct4^{pos} cells from two regions of the mouse brain.

The left panel shows a field of c-kit^{pos} Oct4^{pos} cells from a clone of a cell isolated from the forebrain of an adult mouse. All the cells are strongly positive for Oct4. The right panel shows two Oct4 positive cells from a very early clone of a cell isolated from the paraventricular region.

FIG. 25. Frequency of c-kit^{pos} Oct4^{pos} cells among the Lin^{neg} c-kit^{pos} cells.

Panel E-H shows microscopic images that document the frequency of c-kit^{pos} cells among the small cells isolated from the adult rat myocardium. The nuclei of all the cells are stained in blue with DAPI (panel G). Panel E shows the only c-kit^{pos} cell in the field. Panel F shows the same field stained with an specific antibody against Oct4. There are two Oct4^{pos} cells. Panel H shows the images from panels E to G merged. It is clear that the field contains a single c-kit^{pos} Oct4^{pos} cell.

The right hand graph panel shows the frequency of the c-kit^{pos} Oct4^{pos} cells among the Lin^{neg} c-kit^{pos} cells, which in this example were ˜3%.

FIG. 26. Frequency of c-kit^{pos} Oct4^{pos} cells among the Lin^{neg} c-kit^{pos} cells determined by FACTS sorting.

The bottom panel shows the cytometric histograms of a Lin^{neg} c-kit^{pos} cell population analyzed to identify the Oct4^{pos} cells among them. The upper panel shows the same cell population after cytospin and staining for Oct4.

FIG. 27. A stage-specific cocktail of cardiopoietic growth factors induces CSC cardiospheres to differentiate with high efficiency into the cardiomyocyte lineage and initiate rhythmic beating.

(A) Newly generated cardiospheres are in a primitive state, shown by the expression of stemness markers, such as c-kit (a; green), Oct-4 (b; green), Sox-2 (c; green), Bmi-1 (d; green) and Wnt3a (e; green) as well as showing commitment to the cardiomyocyte lineage by expressing NRx2.5 (f; green). (B) An outline of the stage-specific protocol used for the differentiation of CSC cardiospheres into rhythmic beating cardiomyocytes. (C) At day 8, the cells within the cardiospheres had a changed morphology (a), and immunostaining for c-MHC (red) showed that all the cells within the cardiosphere had differentiated into cardiomyocytes (b). The differentiated cells exhibited sacromeric structures (z lines and dots) within the cell cytoplasm (c; actinin sarcomeric, green) with gap junction formation (c, connexion 43, red). (D) Real-time RT-PCR data showing the fold change of transcripts in differentiated cardiosphere CSCs for c-kit, Oct-4, Gata-4. NRx2.5, cTnI, Cn43, β-MHC and α-MHC following the stage specific cardiomyocyte differentiation protocol.

FIG. 28. Isolation, cloning and characterization of c-kit^{pos} Oct4^{pos} cells from different regions of the adult porcine myocardium.

Panels A and B show a high and low magnification of a c-kit^{pos} Oct4^{pos} cell clone isolated according to the protocol outlined in Examples 15 and 16. Panel C compares the cloning efficiency of c-kit^{pos} Oct4^{pos} cells isolated from the atria, ventricle and apex of a 3 months-old Large White pig compared with the cloning efficiency of mouse and rat myocardial cells. Panel B shows low magnification fields to document that the majority of the cells from the clone shown in panels A and B, express c-kit, Flk-1, Tert, Oct4, Nanog, Isl-1, Bmi-1, and Nkx-2.5. The blow-up squares show a few cells at higher magnification.

FIG. 29. Isolation of c-kit^{pos} Oct4^{pos} cells from the human bone marrow compared with mouse and pig c-kit^{pos} Oct4^{pos} cells isolated from the mouse and pig myocardium.

Human c-kit^{pos} Oct4^{pos} cells isolated from the human bone marrow as described in example 21. All the cells in the field have been labelled with DAPI. The images show that the majority of the c-kit^{pos} cells are also positive for Oct4 using different antibodies. The comparison with the murine and porcine cells emphasizes the similarity of these cell types among different species.

FIG. 30. Stability of the self-renewal and multipotency gene expression phenotype of human cells maintained in culture for up 50 passages (now these cells have reached more than 96 passages).

Panel A-D show the histograms of the cells at the time of their isolation. Panel E shows the partition of the small cells into the different cell populations. Panel F show the pattern of expression of important multipotency and lineage-specific genes at passage 50 at the protein level by immunohistochemistry. Panel G compares the quantitative level of expression at the mRNA level of the same genes shown in panel F. The stability of the phenotype is apparent. Panel H shows the high level of clonability of these cells, an expression of their self-renewal potential. Panel E confirms this high self-renewal as determined by the pseudo-embryoid body formation.

FIG. 31. Karyotype of the human clone shown in FIG. 30 at passage 96.

The karyotype shown in the figure corresponds to a female and both the number of chromosomes, their morphology as well as their G banding pattern (not shown) are normal.

FIG. 32. Optimization of the culture medium for human c-kit^{pos} Oct4^{pos} cells.

The contribution of different growth factors in the growth and maintenance of self-renewal capability of human c-kit^{pos} Oct4^{pos} cells isolated from the adult human myocardium, also known as c-kit^{pos} Lin^{neg} CSCs, was tested by means of cell growth assays (BrdU incorporation, upper left panel), self-renewal capability tested by means of cloning assays (Clonogenesis, upper right panel), loss of self-renewal as indicated by the expression of lineage-specific genes (CTnI positive cells, upper lower panel). The effect of the factors on the expression of multipotency and differentiated genes, respectively, is shown in the lower right panel.

### DETAILED DESCRIPTION OF PREFERRED EMBODIMENTS

### Definitions

As used herein, the following terms and phrases shall have the meanings set forth below. Unless defined otherwise, all technical and scientific terms used herein have the same meaning as commonly understood by one of ordinary skill in the art to which this invention belongs.

The articles “a” and “an” refer to one or to more than one (i.e., to at least one) of the grammatical object of the article. By way of example, “an element” means one element or more than one element.

“Adult” means post-embryonic. With respect to the stem cells of the present invention, the term “adult stem cell” means that the stem cell is isolated from a tissue or organ of an animal at a stage of growth later than the embryonic stage. In one aspect, the stem cells of the invention may be isolated at the post-natal stage. The cells may be isolated preferably from a mammal, such as a human. Adult stem cells are unlike embryonic stem cells, which are defined by their origin, the inner cell mass of the blastocyst. Adult stem cells according to the invention may be isolated from any non-embryonic tissue, and will include neonates, juveniles, adolescents and adult patients. Generally the stem cell of the present invention will be isolated from a non-neonate mammal, and more preferably from a non-neonate human. These adult stem cells are characterized in that, in their undifferentiated state, they express telomerase, and they do not show gap junctional intercellular communication (GJIC) and do not have a transformed phenotype.

A “biocompatible implant” is any article intended for implantation, which is considered to be suitable for such implantation, and is considered unlikely to cause an adverse reaction. By “likely to cause an adverse reaction” is intended to mean that the article will cause an adverse reaction following implantation in less than about 70% of cases. In some embodiments, the article will cause an adverse reaction in less than about 80%, less than about 85%, less thank about 90%, less than about 95%, less than about 99% or more of cases. By way of example, a biocompatible implant may include, an autologous or allogeneic organ or tissue.

By “cardiac tissue” is meant any tissue that is present within the heart of a subject. Such cardiac tissue includes myocardium. Such cells may comprise a primary cell culture or an immortalized cell line. The cardiac tissue may be from any organism possessing cardiac tissue. Preferably, the cardiac tissue is mammalian; more preferably the cardiac tissue is human. Cardiac tissue cells can be isolated, for example, from the hearts of sacrificed animals, from small cardiac biopsies obtained during cardiac surgery, or by means of a biopsy catheter during cardiac surgery. The source of cardiac tissue or the method of isolation of the cardiac tissue is not critical to the invention.

The term “cellular composition” refers to a preparation of cells, which preparation may include, in addition to the cells, non-cellular components such as cell culture media, e.g. proteins, amino acids, nucleic acids, nucleotides, co-enzyme, anti-oxidants, metals and the like. Furthermore, the cellular composition can have components which do not affect the growth or viability of the cellular component, but which are used to provide the cells in a particular format, e.g., as polymeric matrix for encapsulation or as a pharmaceutical preparation.

The term “chromatic rearrangement” is intended to cover any rearrangement of the chromosomal structure, which allows the chromosomal structure to differ from the normal, expected chromosomal structure. By way of example, the term encompasses chromosome translocation, chromosomal breakage and chromosome multiplication.

The term “clonogenic” relates to the clonal proliferation capacity of the cells of the invention. The term is intended to convey that the cells proliferate by dividing to form clones, which further divide to form more clones, and in this way to increase in number and expand the cell population.

The terms “comprise” and “comprising” are used in the inclusive, open sense, meaning that additional elements may be included.

The term “culture” refers to any growth of cells, organisms, multicellular entities, or tissue in a medium. The term “culturing” refers to any method of achieving such growth, and may comprise multiple steps. The term “further culturing” refers to culturing a cell, organism, multicellular entity, or tissue to a certain stage of growth, then using another culturing method to bring the cell, organism, multicellular entity, or tissue to another stage of growth. A “cell culture” refers to a growth of cells in vitro. In such a culture, the cells proliferate, but they may not organize into a tissue per se. A “tissue culture” refers to the maintenance or growth of tissue, e.g., explants of organ primordial or of an adult organ in vitro so as to preserve its architecture and function. A “monolayer culture” refers to a culture in which cells multiply in a suitable medium while being principally attached to each other and to a substrate. Furthermore, a “suspension culture” refers to a culture in which cells multiply while suspended in a suitable medium. Likewise, “conditioned media” refers to the cultivation of cells or explants in a continuous flow of fresh medium to maintain cell growth, e.g. viability. In one aspect said cells may be stem cells and in another aspect the cells may be embryonic stem cells or the cells of the invention. The term “conditioned media” refers to the supernatant, e.g. free of the cultured cells/tissue, resulting after a period of time in contact with the cultured cells such that the media has been altered to include certain paracrine and/or autocrine factors produced by the cells and secreted into the culture. A “confluent culture” is a cell culture in which all the cells are in contact and thus the entire surface of the culture vessel is covered, and implies that the cells have also reached their maximum density, though confluence does not necessarily mean that division will cease or that the population will not increase in size thereafter.

The term “culture medium” or “medium” is recognized in the art, and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for bacterial culture is a medium. Similarly, a powder mixture that when mixed with water or other liquid becomes suitable for cell culture may be termed a “powdered medium”. “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the invention while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (IMDM).

In one aspect the growth media may be any one of Media I-IV, as these are described herein.

The media may be medium I, which comprises 10% embryonic stem cell-qualified fetal bovine serum (ES-FBS, Invitrogen); 10 ng/ml mouse basic fibroblast growth factor (FGFb, PeproTech), 20 ng/ml mouse endothelial growth factor (EGF, PeproTech), 10 ng/ml mouse leukaemia inhibitory factor (LIF, Chemicon); 6.7 ng/ml sodium selenite, 10 μg/ml insulin, 5.5 μg/ml transferring, 2 μg/ml ethanolamine (ITS, Invitrogen); 50 μg/ml gentamycin, 0.1 mg/ml streptomycin and 100 U/ml penicillin (Sigma); 250 ng/ml amphotericin B, and 205 ng/ml sodium deoxycholate (Fungizone, Invitrogen) in 1:1 Dulbecco's Modified Eagle's Medium/Nutrient Mixture F-12 Ham (DMEM/F12, Sigma).

The media may be medium II, which is the same as Medium I, but wherein the serum is depleted of differentiation factors and other high molecular weight proteins by treatment with DCC solution, prepared as follows: 0.45 g of dextran T500 and 4.5 g activated charcoal (Sigma) were stirred overnight at 4° C. in 1800 ml 0.01M Tris-HCl (Sigma), pH 8.0 in a tightly closed Erlenmeyer bottle. DCC solution was centrifuged at 2000 g for 20 min in 50 ml plastic tubes, the supernatant was discarded and new DCC solution was added to the same tubes and centrifuged again, in order to obtain “double pellets”. After inactivating the FBS 30 min at 56° C., 50 ml of FBS was mixed with each double pellet and transferred to a glass bottle, incubating the mixture for 45 min at 45° C. under shaking. Afterwards, the mixture was centrifuged 20 min at 2000 g and the supernatant was mixed with a new DCC double pellet and incubated again 45 min at 45° C. in a glass bottle under shaking. After centrifuging 20 mM at 2000 g, the FBS supernatant was sterilized through a 0.22 μm low protein binding filter.

The media may be medium III which comprises 10 ng/ml mouse basic fibroblast growth factor (FGFb, PeproTech), 10 ng/ml mouse endothelial growth factor (EGF, PeproTech), 10 ng/ml mouse leukaemia inhibitory factor (LIF, Chemicon); 0.1 mM 2-mercaptoethanol, 1 mM L-glutamate, 15 nM sodium selenite, 25 μg/ml BSA (Sigma); 0.5× Bottenstein's N-2 supplement, 0.5×B27 supplement without vitamin A (Invitrogen); 50 μg/ml gentamycin, 0.1 mg/ml streptomycin and 100 U/ml penicillin (Sigma); 250 ng/ml amphotericin B, and 205 ng/ml sodium deoxycholate (Fungizone, Invitrogen) in 1:1 Neurobasal (Invitrogen) and DMEM/F12 (Sigma) media.

The media may be medium IV, which comprises 10% embryonic stem cell-qualified fetal bovine serum (ES-FBS), 5% horse serum (Invitrogen); 10 ng/ml mouse basic fibroblast growth factor (FGFb, PeproTech), 20 ng/ml mouse endothelial growth factor (EGF, PeproTech), 10 ng/ml mouse leukaemia inhibitory factor (LIF, Chemicon); 5 mU/ml erythropoietin, 50 μg/ml porcine gelatin, 0.2 mM L-glutathione, 50 μg/ml gentamycin, 0.1 mg/ml streptomycin and 100 U/ml penicillin (Sigma); 250 ng/ml amphotericin B, and 205 ng/ml sodium deoxycholate (Fungizone, Invitrogen) in F-12K nutrient mixture with Kaighn's modification (Invitrogen), pH 7.4.

Differentiation medium, also known as embryoid body formation medium, may be a complex medium designed to trigger the commitment of the cells of the invention to the differentiation pathway and to downregulate their self-renewal. In one aspect, the differentiation medium may be differentiation medium I (DMI) or differentiation medium II (DMII) supplemented with specific differentiation factors. DMI contains 20% FCS, 2 mM L-glutamine, 1×MEM non-essential aminoacids, 0.1 mM (3-ME, 50 μg/ml gentamycin, 0.1 mg/ml streptomycin, 100 U/ml penicillin (Sigma), 250 ng/ml amphotericin B, and 205 ng/ml sodium deoxycholate (Fungizone, Invitrogen) in Dulbecco's medium (DMEM, Gibco). DM II contains 15% DCC-treated FBS, 1×ITS supplement (Invitrogen), 2 mM L-glutamine, 1×MEM non-essential aminoacids, 0.1 mM β-ME, 50 μg/ml gentamycin, 0.1 mg/ml streptomycin, 100 U/ml penicillin (Sigma), 250 ng/ml amphotericin B, and 205 ng/ml sodium deoxycholate (Fungizone, Invitrogen) in Dulbecco's medium (DMEM, Gibco).

“Dedifferentiation” refers to the loss of characteristics of a specialized cell, and its regression into an undifferentiated or less differentiated state. The dedifferentiated cell may become redifferentiated into a cell of the same cell type as before the dedifferentiation, or into a cell of a different type.

The term “differentiation” refers to the formation of cells expressing markers known to be associated with cells that are more specialized and closer to becoming terminally differentiated cells that are incapable of further division or differentiation. For example, in a pancreatic context, differentiation might be seen in the production of islet-like cell clusters containing an increased proportion of beta epithelial cells that produce increased amounts of insulin. The terms “further” or “greater” differentiation refers to cells that are more specialized and closer to becoming terminally differentiated cells incapable of further division or differentiation than the cells from which they were cultured. The term “final differentiation” refers to cells that have become terminally differentiated cells incapable of further division or differentiation.

An “embryoid body” or a “pseudo-embryoid body”, which in this application are used as synonyms, is an aggregate of cells that under the culture conditions given in the application start to differentiate into different cell types. Preferably said cells are the adult stem cells of the invention.

An “embryonic stem cell” is a totipotent cell isolated from a very early embryo. These cells are not differentiated and have the capacity to differentiate into endoderm, ectoderm and endoderm, and further to differentiate into any of the cells in the body. The embryonic stem cell is generally isolated from a very early mammalian embryo, such as a human embryo.

The terms “Oct4 cell” is used to describe the c-kit^{pos} adult stem cells of the invention which, in addition to Oct4, may also express many other genes which characterize the multipotent state. These cells, which may be isolated from cardiac tissue, when isolated from this source are referred like “Oct-4 CSCs” or “CSCs”—Cardiac Stem Cells—)

The term “expressed” is used to describe the presence of a marker within a cell. In order to be considered as being expressed, a marker must be present at a detectable level. By “detectable level” is meant that the marker can be detected using one of the standard laboratory methodologies such as PCR, blotting or FACS analysis. A gene is considered to be expressed by a cell of the population of the invention if expression can be reasonably detected after 30 PCR cycles, which corresponds to an expression level in the cell of at least about 100 copies per cell. The terms “express” and “expression” have corresponding meanings. At an expression level below this threshold, a marker is considered not to be expressed. The comparison between the expression level of a marker in an adult stem cell of the invention, and the expression level of the same marker in another cell, such as for example an embryonic stem cell, may preferably be conducted by comparing the two cell types that have been isolated from the same species. Preferably this species is a mammal, and more preferably this species is human. Such comparison may conveniently be conducted using a reverse transcriptase polymerase chain reaction (RT-PCR) experiment.

“Fluorescence activated cell sorting (FACS)” is a method of cell purification based on the use of fluorescent labeled antibodies. The antibodies are directed to a marker on the cell surface, and therefore bind to the cells of interest. The cells are then separated based upon the fluorescent emission peak of the cells.

“Gap junction intercellular communication” is a mechanism of exchange of small molecules, such as intracellular signaling molecules, and ions between cells. The small molecules and ions are exchanged through gap junctions; structures which connect the cytoplasm of adjacent cells.

“Immuno-affinity purification” is a method of cell purification using immobilized antibodies directed to a marker on the cell surface. The sample is applied to a column containing the immobilized antibodies, and the cells of interest are bound by the immobilized antibody. Following a washing step, the cells of interest are eluted from the column using a competitor with higher affinity for the immobilized antibody.

The term “including” is used herein to mean “including but not limited to”. “Including” and “including but not limited to” are used interchangeably.

The term “isolated” indicates that the cell or cell population to which it refers is not within its natural environment. The cell or cell population has been substantially separated from surrounding tissue. In some embodiments, the cell or cell population is substantially separated from surrounding tissue if the sample contains at least about 75%, in some embodiments at least about 85%, in some embodiments at least about 90%, and in some embodiments at least about 95% adult stem cells. In other words, the sample is substantially separated from the surrounding tissue is the sample contains less than about 25%, in some embodiments less than about 15%, and in some embodiments less than about 5% of materials other than the adult stem cells. Such percentage values refer to percentage by weight. The term encompasses cells which have been removed from the organism from which they originated, and exist in culture. The term also encompasses cells which have been removed from the organism from which they originated, and subsequently re-inserted into an organism. The organism which contains the re-inserted cells may be the same organism from which the cells were removed, or it may be a different organism.

“Marker” refers to a biological molecule whose presence, concentration, activity, or phosphorylation state may be detected and used to identify the phenotype of a cell.

A “medical device intended for implantation” is any artificial medical device which is intended to be implanted into a patient. Such devices may be intended for implantation into any part of the patient's body. The device may have any one or more of a number of functions, including but not limited to, providing structural support, repairing damaged tissue and maintaining natural and exogenous components in the correct position and orientation within the patient's body.

The term “multipotent” refers to a cell which is capable of giving rise to multiple different types of cell. Specifically, the term refers to a cell which is able to differentiate into cell types of mesodermal, endodermal and ectodermal origin.

“Natural expression” refers to the endogenous expression of one or more genes in a cell. For expression to be considered natural, the cell will express the gene without the need for any recombinant manipulation to introduce the gene or any of its regulatory elements into the cell or to modulate these genes' expression by introduction of exogenous genetic material. The term “recombinant manipulation” refers to any sort of manipulation of the genetic material contained within the cell, wherein genetic material is combined with other genetic material with which it is not naturally associated. This includes, by way of example only, gene insertion, gene deletion, and insertion of a heterologous (non-natural) or stronger promoter or other regulatory element operably linked to either the endogenous gene or an exogenously introduced version of the gene, including insertion of an exogenous promoter or regulatory element, or insertion of an endogenous promoter or regulatory element at a position at which it would not be expected to occur. The naturally expressed gene will not contain or be associated with any heterologous sequences, and in particular, will not contain any retroviral sequences, whether promoter sequences, regulatory sequences, or otherwise.

Natural expression is from genomic DNA within the cells, and so each gene that is naturally expressed may include introns between the exons within its coding sequence. The naturally expressed gene will show an intron-exon structure which is identical to that found within a non-manipulated cell. Natural expression is not from cDNA. Natural expression can if necessary be proven by any one of various methods, such as sequencing out from within the reading frame of the gene to check that no extraneous heterogenous sequence is present. The copy number of the gene can also be checked as the natural copy number, for example, using a technique such as fluorescence in situ hybridisation. The gene will thus be present in the genome in its natural genome context, and the histone condensation state will be such as to allow appropriate expression of the gene.

The terms “naturally expressing”, “naturally expresses”, and “naturally expresses” have their corresponding meanings.

The term “passage” refers to a method of sub-culturing cells. Passaging is required when a large number of cells are being grown, as without it the cells would exhaust the nutrient supply of the media, become compressed against each other and die. Generally, cells are grown in a flask or dish with a supply of nutrient media, where they adhere to the bottom of the dish, and can become confluent in 2-3 days. In order to passage the cells, the media is removed and the cells are generally washed before being treated with trypsin to reduce their adherence to the surface on which they are grown. The cells are then suspended in a liquid, generally PBS, before an appropriate number of cells are transferred to a new flask or dish.

A “patient”, “subject” or “host” to be treated by the method of the invention may mean either a human or non-human animal and is preferably a mammal, more preferably a human.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. The term “phenotype” refers to the observable characteristics of a cell, such as size, morphology, protein expression, etc.

The term “totipotent” refers to a cell which, when placed into the proper environment (e.g. an early blastocyst) is capable of generating a complete and viable new individual completely derived from this cell.

The term “pluripotent” refers to cells which are capable of differentiating into a number of different cell types. The term also implies that all the progeny of a pluripotent cell correspond to derivates of a single embryonic cell layer. These cells do not necessarily have to possess a tripotent capacity; the capacity to differentiate into mesoderm, endoderm and ectoderm, but they are not terminally differentiated, and therefore maintain the capacity to differentiate into a number of different cell types.

In the context of this application the term “tripotent” refers to a cell which, although it may not be totipotent, is capable of generating cell types corresponding to the three layers of the early embryo; mesoderm, endoderm and ectoderm.

The term “progenitor cell” refers to a cell that has the capacity to create progeny that are more differentiated than itself. For example, the term may refer to an undifferentiated cell or a cell differentiated to an extent short of final differentiation which is capable of proliferation and giving rise to more progenitor cells having the ability to generate a large number of mother cells that can in turn give rise to differentiated, or differentiable daughter cells. In a preferred embodiment, the term progenitor cell refers to a generalized mother cell whose descendants (progeny) specialize, often in different directions, by differentiation, e.g., by acquiring completely individual characters, as occurs in progressive diversification of embryonic cells and tissues. A progenitor cell is more differentiated than a true stem cell and has already somewhat restricted the multipotent capacity of the true stem cell. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. However, it will be apparent to one skilled in the art that the cells of the present invention naturally possess a tripotent capacity, without recombinant manipulation. By this definition, stem cells may also be progenitor cells, as well as the more immediate precursors to terminally differentiated cells.

“Proliferation” refers to an increase in cell number. “Proliferating” and “proliferation” refer to cells undergoing mitosis.

The term “recombinant manipulation” refers to any sort of manipulation of the genetic material contained within a cell. This includes, by way of example only, gene insertion, gene deletion, and insertion of a promoter or other regulatory element into a cell, including insertion of an exogenous promoter or regulatory element, or insertion of an endogenous promoter or regulatory element at a position at which it would not be expected to occur.

The term “self-renewing” should be understood to represent the capacity of a cell to reproduce itself whilst maintaining the original proliferation and differentiation properties of cells of the invention. Such cells proliferate by dividing to form clones, which further divide into clones and therefore expand the size of the cell population without the need for external intervention, without evolving into cells with a more restricted differentiation potential.

The “side population” is a sub-population of cells distinguished from the main population of cells by one or more markers employed to separate these cells. The side population can generally be distinguished through flow cytometry or fluorescence activated cell sorting (FACS) analysis, and by definition includes cell which have distinguishing biological characteristics from the rest of the cell population.

As used herein, the term “solution” includes a pharmaceutically acceptable carrier or diluent in which the cells of the invention remain viable.

The term “substantially pure” as used herein, refers to a population of stem cells that is at least about 75%, in some embodiments at least about 85%, in some embodiments at least about 90%, and in some embodiments at least about 95% pure, with respect to other cells that make up a total cell population. For example, with respect to cardiac tissue-derived stem cell populations, this term means that there are at least about 75%, in some embodiments at least about 85%, in some embodiments at least about 90%, and in some embodiments at least about 95% pure, cardiac stem cells compared to other cells that make up a total cell population. In other words, the term “substantially pure” refers to a population of stem cells of the present invention that contain fewer than about 25%, in some embodiments fewer than about 15%, and in some embodiments fewer than about 5%, of lineage committed cells in the original unamplified and isolated population prior to subsequent culturing and amplification.

“Support” as used herein refers to any device or material that may serve as a foundation or matrix for the growth of cardiac tissue-derived stem cells.

“Telomerase” is the enzyme responsible for adding telomeric repeats to the telomeres which are situated at the ends of eukaryotic chromosomes. The role of telomerase is to solve the end replication problem, and to prevent the progressive shortening of telomeres, which leads towards senescence. Telomerase is not generally activated in adult somatic cells.

“Therapeutic agent” or “therapeutic” refers to an agent capable of having a desired biological effect on a host. Chemotherapeutic and genotoxic agents are examples of therapeutic agents that are generally known to be chemical in origin, as opposed to biological, or cause a therapeutic effect by a particular mechanism of action, respectively. Examples of therapeutic agents of biological origin include growth factors, hormones, and cytokines. A variety of therapeutic agents are known in the art and may be identified by their effects. Certain therapeutic agents are capable of regulating cell proliferation and differentiation. Examples include chemotherapeutic nucleotides, drugs, hormones, non-specific (non-antibody) proteins, oligonucleotides (e.g., antisense oligonucleotides that bind to a target nucleic acid sequence (e.g., mRNA sequence)), peptides, and peptidomimetics.

“Tissue regeneration” is the process of increasing the number of cells in a tissue following a trauma. The trauma can be anything which causes the cell number to diminish. For example, an accident, an autoimmune disorder or a disease state could constitute trauma. Tissue regeneration increases the cell number within the tissue and enables connections between cells of the tissue to be re-established, and the functionality of the tissue to be regained.

The term “totipotent” refers a stem cell with the capacity to differentiate into a cell of any cell type. Embryonic stem cells are totipotent. The cells of the invention are not embryonic stem cells.

The term “tripotent” refers to a stem cell with the capacity to differentiate into a cell from the mesoderm, endoderm and ectoderm cell layers. Cells according to the invention are considered tripotent, and are capable of differentiating into at least one cell type of each of an endodermal type, an ectodermal cell type and a mesodermal cell type, without recombinant manipulation of the cells.

By “tropism” is meant the ability of the cell to home toward a particular tissue, organ or area. For example, the cells of the invention may show preferential tropism for their organ of origin.

Tripotent Capacity

In one aspect of the invention, the adult stem cell population is characterised in that the cells have tripotent capacity or potential. As defined above, this tripotent potential allows the cells to develop into cells derived from the endoderm, mesoderm or ectoderm. In certain aspects, the adult stem cell population is tripotent if the cells of the adult stem cell population are capable of differentiating into at least one cell type of each of an endodermal type, an ectodermal cell type and a mesodermal cell type without recombinant manipulation of the cells. In certain embodiments, the cell population is considered to have tripotent potential if at least about 70% of the cells of the isolated adult stem cell population show tripotent capacity. In other embodiments, at least about 80%, at least about 90% or at least about 95% of the cells of the adult stem cell population show tripotent potential. In yet other embodiments, at least about 99% or even 100% of the cell population show tripotent potential. Tripotent potential can be determined by forming the cells into embryoid bodies and culturing the embryoid bodies in specific differentiation media. The cells can then be amplified and differentiation confirmed by quantitative PCR, using lineage-restricted transcripts. It should be noted that none of the adult stem cells known until now are “tripotent” or even “bipotent”.

The cells of the invention are able to remain in culture as undifferentiated cells through a number of passages, and do not differentiate until they are provided with appropriate differentiating media. Upon administration of appropriate differentiating media, the cells of the invention are capable of differentiating into any one of mesoderm, ectoderm or endoderm.

Indeed, the cells of the invention can be induced to differentiate into any cell type upon addition of the appropriate differentiation media. Specifically, the adult stem cells of the invention have the ability to differentiate into any of the tissues found within the animal from which the adult stem cells were isolated. In some embodiments, the stem cell population is considered to have the potential to differentiate into any tissue if at least about 70% of the cells of the isolated adult stem cell population have this ability. In other embodiments, at least about 80%, at least about 90% at least about 95%, 99% or even 100% of the cell population should show the potential to differentiate into any tissue. As described previously, the ability of an isolated adult stem cell population to differentiate into any tissue can be measured by culturing a portion of the isolated adult stem cell population in appropriate differentiating media for production of a specific tissue. Quantitative PCR using lineage-derived transcript amplification can then be conducted to ascertain whether differentiation has occurred.

In one aspect, the cells of the isolated adult stem cell population have the capacity to differentiate into any tissue cell-type of the body. Within a further aspect of the invention, the cells of the isolated adult stem cell population have the capacity to differentiate into cardiac tissue, spleen tissue, bone marrow, lung tissue, skin, intestinal tissue, liver tissue, brain tissue and skeletal muscle. This list is provided by way of illustration only, and is not intended to be exhaustive. It will be understood by one skilled in the art that in order to induce differentiation of the isolated adult stem cells into a specific tissue type, a tissue specific differentiation media may be required. For example, in order to induce the cells of the invention to differentiate into bone marrow, a bone marrow specific differentiation media may be required. It will be further appreciated by one of skill in the art that in order to detect successful differentiation into a specific tissue type, a lineage-derived transcript must be detected. For example, in order to detect differentiation into bone marrow, a bone marrow-derived transcript might be amplified, for example using specific primers during a quantitative PCR experiment.

One of the characteristic features of the cell population of the invention is the ability to culture the cells in basic medium for a prolonged period of time without differentiation occurring. In one aspect of the invention, the cell population can be grown in basic medium without becoming differentiated.

The stem cell population of the invention can be passaged at least 100 times, in some embodiments at least 200 times, and in some embodiments at least 300 times, in basic media without undergoing differentiation from a cell type with potential for tripotency.

In terms of exposure time, the cell population of the invention can be passaged in basic media for a number of months, and in some embodiments years without undergoing differentiation. In some embodiments at least, the cell population of the invention can be passaged for a period of at least 1 year, in some embodiments at least 2 years, and in some embodiments at least 3 years, without undergoing differentiation. The ability to grow without undergoing differentiation means that the isolated adult stem cell population retains its tripotent capacity, as defined above.

The cell population of the invention can be cultured in basic media without undergoing chromatographic rearrangement during the passaging steps. In some embodiments at least, the cells are considered not to have undergone chromatographic rearrangement if at least about 70% of the cells of the isolated adult stem cell population do not show any chromatographic rearrangement. In some embodiments at least about 80%, in some embodiments at least about 90%, in some embodiments at least about 95%, and in some embodiments at least 99% or more of the cells of the cell population do not show any chromatographic rearrangement. Chromatographic rearrangement can be conveniently detected by means of producing a karyotype image of the chromosomes of a cultured cell of the invention, and comparing this with a freshly isolated cell of the invention.

In certain embodiments, the cells of the population of the invention are clonogenic. In some embodiments, the isolated adult stem cell population is considered to be clonogenic if at least about 70% of the cells of the isolated adult stem cell population are clonogenic. In some embodiments at least about 80%, in some embodiments at least about 90%, in some embodiments at least about 95%, and in some embodiments at least 99% or more of the cells of the isolated adult stem cell population are clonogenic.

Cell Markers

The invention provides a population of isolated adult stem cells, wherein the cell population essentially comprises only cells of the invention, i.e. the cell population is pure. In many aspects, the cell population comprises at least about 80% (in other aspects at least 85%, 90%, 91%, 92%, 93%, 94%, 95%, 96%, 97%, 98%, 99%, 99.5%, 99.9% or 100%) of the adult stem cells of the invention.

The isolated adult stem cell of the invention is characterised in that it has a distinctive expression level for certain markers, some of which have previously been used to denote totipotency, and is distinguished from embryonic stem cells. It is shown herein that the isolated adult stem cells of the invention express many markers at a detectable level, but at a level lower than their expression in embryonic stem cells and so these adult stem cells are without any doubt different from embryonic stem cells.

The adult stem cell population of the invention is considered to express a marker if at least about 70% of the cells of the population show detectable expression of the marker. In other aspects, at least about 80%, at least about 90% or at least about 95% or at least about 97% or at least about 98% or more of the cells of the population show detectable expression of the marker. In certain aspects, at least about 99% or 100% of the cells of the population show detectable expression of the markers. Expression may be detected through the use of an RT-PCR experiment or through fluorescence activated cell sorting (FACS). It should be appreciated that this list is provided by way of example only, and is not intended to be limiting.

The markers described below are considered to be expressed by a cell of the population of the invention, if expression can be reasonably detected after 30 PCR cycles, which corresponds to an expression level in the cell of at least about 100 copies per cell.

In a primary embodiment, the invention relates to an adult stem cell population characterised in that the cells of the population express one or more of the markers c-kit, Nanog and Oct-4.

The term c-kit includes c-kit and any orthologs thereof, included but not limited to CD117, Fdc, Gsfsco1, Gsfsco5, Gsfsow3, SCO1, SCO5, SOW3, Ssm, Tr-kit, and KIT.

The term Nanog includes Nanog and any orthologs thereof, including but not limited to 2410002E02Rik, ENK, ecat4 homeobox transcription factor Nanog, and homeobox transcription factor Nanog-delta 48.

The term Oct-4 includes Oct-4 and any orthologs thereof, including but not limited to Pou5f1, POU domain class 5 transcription factor 1, Oct-3, Oct-3/4, Oct3, Otf-3, Otf-4, Otf3-rs7, and Otf3 g.

In certain embodiments, the cell population is further characterised in that the cells express one or more of the markers c-kit, Nanog and Oct-4 at a detectable level, but at a level lower than the expression level of these markers in embryonic stem cells. In some embodiments, the cells express one or more of c-kit, Nanog and Oct-4 at a level which is between ⅓ and 1/10 of the level of their expression in embryonic stem cells, although in some isolates the average expression level may be lower or higher than this value. In each isolate there may be cells with a marker expression level that is higher than expression level in an average embryonic stem cell, although in most cells the expression level of the markers is lower than the expression level in embryonic stem cells. The comparison between the expression level of the markers in an adult stem cell of the invention, and the expression level of the markers in an embryonic stem cell may be conducted by comparing an adult stem cell and an embryonic stem cell that have been isolated from the same species. Preferably this species is a mammal, and more preferably this species is human. Such comparison may conveniently be conducted using a reverse transcriptase polymerase chain reaction (RT-PCR) experiment.

The cells of the population of the invention may express c-kit at a level which is lower than the level of expression of c-kit in an embryonic stem cell of the same species. In some embodiments, the cells may express c-kit at a level of between 10⁻³ and 10⁻⁶ mRNA copies per cell relative to the expression level of the protein GAPDH. The normalisation of the expression level relative to GAPDH is a procedure well known to those skilled in the art.

The cells of the population of the invention may also express Nanog at a level which is lower than the level of expression of Nanog in an embryonic stem cell. In some embodiments, the cells of the invention may express Nanog at a level of between 10⁻² and 10⁻³ mRNA copies per cell relative to GAPDH.

The cells of the population of the invention may also express Oct-4 at a level which is lower than the level of expression of Oct-4 in an embryonic stem cell. In some embodiments, the cells of the invention may express Oct-4 at a level of between 10⁻³ and 10⁻⁴ mRNA copies per cell relative to GAPDH.

In a further embodiment, the cell population of the invention may be characterised in that the cells of the isolated adult stem cell population may also express one, two, three or all of the markers Rex1, Mph1, Eed, SSEA-1 and Mlc2a, at a level lower than the level of expression of these markers in embryonic stem cells.

The term Rex1 includes Rex-1 and any orthologs thereof, including but not limited to ZFP42, zinc finger protein 42 homolog, ZNF754, REX1 transcription factor and zinc finger protein 42.

The term Mph1 includes Mph1 and any orthologs thereof, including but not limited to YIR002C and Mph1p.

The term Eed includes Eed and any orthologs thereof, including but not limited to embryonic ectoderm development, 1(7)5Rn, 17Rn5, lusk, lethal, Chr 7, and Rinchik 5.

The term Mlc2a includes Mlc2a and any orthologs thereof, including but not limited to My17, myosin light polypeptide 7, MLC-2alpha, MLC2a, MYL2A, Mylc2a, and RLC-A. In some embodiments, the cells express Rex1, Mph1, Eed and Mlc2a at a level which is at least 10 times, 100 times or even 1000 times less than the level of their expression in embryonic stem cells.

In a further embodiment, the cell population of the invention may be characterised in that the cells of the isolated adult stem cell population also express one, two, three, four, five, six, seven, eight, nine or all ten of the markers MDR-1, TERT, CD133, Gata-4, Gata-6, SOX-2, klf-4, c-myc, CD90, CD166 and Bmi-1, at a level lower than the level of expression of these markers in embryonic stem cells.

The term MDR-1 includes MDR-1 and any orthologs thereof, including but not limited to ABCB1, ATP-binding cassette sub-family B (MDR/TAP), ABC20, CD243, CLCS, GP170, MDR1, MGC163296, and P-gp, PGY1.

The term TERT includes TERT and any orthologs thereof, including but not limited to telomerase reverse transcriptase, EST2, TCS1, TP2, TRT, hEST2, and telomerase catalytic subunit.

The term CD133 includes CD133 and any orthologs thereof, including but not limited to prominin 1, AC133, MSTP061, PROML1, hProminin, hematopoietic stem cell antigen, and prominin-like 1.

The term Gata-4 includes Gata-4 and any orthologs thereof, including but not limited to GATA-4 zinc-finger transcription factor.

The term Gata-6 includes Gata-6 and any orthologs thereof, including but not limited to transcription factor GATA-6, and MGC79905.

The term SOX-2 includes Sox-2 and any orthologs thereof, including but not limited to K08A8.2.

The term klf-4 includes klf-4 and any orthologs thereof, including but not limited to Kruppel-like factor 4, EZF, GKLF, and endothelial Kruppel-like zinc finger protein.

The term c-myc includes c-myc and any orthologs thereof, including but not limited to Myc, myelocytomatosis oncogene, AU016757, Myc2, Niard, Nird, c-myc proto-oncogene, and myc proto-oncogene protein.

The term CD90 includes CD90 and any orthologs thereof, including but not limited to Thy1, thymus cell antigen 1, theta, T25, Thy-1, Thy-1.2, Thy1.1, and Thy 1.2.

The term CD166 includes CD166 and any orthologs thereof, including but not limited to Alcam, activated leukocyte cell adhesion molecule, AI853494, BEN, DM-GRASP, MGC27910, MuSC, and SC1.

The term Bmi-1 includes Bmi-1 and any orthologs thereof, including but not limited to Bmi1 polycomb ring finger oncogene, RP23-396N6.2, AW546694, Bmi-1, Pcgf4, B lymphoma Mo-MLV insertion region 1; polycomb group ring finger 4.

In a further embodiment, the cell population of the invention may be characterised in that the cells of the isolated adult stem cell population also express one, two, three, four, five, six, seven, eight, nine, ten or all eleven of the markers Isl-1, FoxD3, Mel-18, M33, Mph1/Rae-28, SDF1/CXCL12, BMP2, BPM-4, Wnt-3A, Wnt-4, and Wnt-11, in some variations, at a level lower than the level of expression of these markers in embryonic stem cells.

The term Isl-1 includes Isl-1 and any orthologs thereof, including but not limited to Inhibitor of Serine protease Like protein, and R10H1.4.

The term FoxD3 includes FoxD3 and any orthologs thereof, including but not limited to forkhead box D3, fkd6, fkh6, forkhead-6, zgc:111934, fork head domain protein 6, and mother superior.

The term Mel-18 includes Mel-18 and any orthologs thereof, including but not limited to PCGF2, polycomb group ring finger 2, MGC10545, RNF110, ZNF144, ring finger protein 110, and zinc finger protein 144.

The term M33 includes M33 and any orthologs thereof, including but not limited to Cbx2, chromobox homolog 2, RP23-458A23.7, MOD2, pc, M33 polycomb-like protein; chromobox homolog 2, and homobox homolog 2.

The term Mph1/Rae-28 includes Mph1, Rae-28, and any orthologs thereof, including but not limited to Phc1, polyhomeotic-like 1, Edr, Edr1, and AW557034.

The term SCF1/CXCL12 includes SCF1, CXCL12 and any orthologs thereof, including but not limited to chemokine (C-X-C motif) ligand 12 (stromal cell-derived factor 1), PBSF, SCYB12, SDF-1a, SDF-1b, SDF1, SDF1A, SDF1B, TLSF-a, TLSF-b, TPAR1, stromal cell-derived factor 1 delta, stromal cell-derived factor 1 gamma, and stromal cell-derived factor 1a. The term BMP2 includes BMP2 and any orthologs thereof, including but not limited to bone morphogenetic protein 2 and BMP2A.

The term BMP-4 includes BMP-4 and any orthologs thereof, including but not limited to bone morphogenetic protein 4, MGC100779, bmp-4, zbmp-4, zgc:100779, and etID309887.17.

The term Wnt-3A includes Wnt-3A and any orthologs thereof, including but not limited to wingless-type MMTV integration site family, member 3, WNT-3A, Wnt-3a homolog; Wnt3a variant 3, wingless-type MMTV integration site family member 3a, and wingless-type MMTV integration site family member 3A.

The term Wnt-4 includes Wnt-4 and any orthologs thereof, including but not limited to wingless-type MMTV integration site family member 4, RP1-224A6.7, SERKAL, WNT-4, OTTHUMP00000044725, and WNT-4 protein.

The term Wnt-11 includes Wnt-11 and any orthologs thereof, including but not limited wingless-type MMTV integration site family member 11.

The cell population of the invention may also be characterised in that the cells do not express a particular selection of markers at a detectable level. Many of these are indicative of a differentiated or partially differentiated cell. As defined herein, these markers are said be to be negative markers.

In some embodiments, the stem cell population of the invention is considered not to express a marker if at least about 70% of the cells of the isolated adult stem cell population should not show detectable expression of the marker. In other embodiments, at least about 80%, at least about 90% or at least about 95% or at least about 97% or at least about 98% or at least about 99% or 100% of the cells of the stem cell population should not show any detectable expression of the marker. Again, lack of detectable expression may be proven through the use of an RT-PCR experiment or using FACS.

The markers described above are considered not to be expressed by a cell population of the invention, if expression cannot be reasonably detected at a level of 30 cycles of PCR, which corresponds to an expression level in the cell of less than about 100 copies per cell.

In one embodiment, the cell population is further characterised in that the cells do not express one, two, three, four, five, six, seven, eight, nine, ten, eleven, twelve, or all thirteen of the markers Cd11b, CD13, CD14, CD29, CD31, CD33, CD36, CD38, CD49f, CD62, CD73, CD105, and CD106 at a detectable level. As described above, it is possible for these markers not to be expressed despite a small amount of residual expression persisting.

The term cd11b includes cd11b and any orthologs thereof, including but not limited to Itgam, integrin alpha M, CD11b/CD18, CR3, CR3A, F730045J24Rik, Ly-40, MAC1, Mac-1, Mac-1a, CD11B (p170); Mac-1 alpha; cell surface glycoprotein MAC-1 alpha subunit; complement component receptor 3 alpha, complement component receptor 3 alpha-a, complement receptor type 3, leukocyte adhesion receptor M01, and macrophage antigen alpha.

The term CD13 includes CD13 and any orthologs thereof, including but not limited to ANPEP, alanyl (membrane) aminopeptidase (aminopeptidase N, aminopeptidase M, microsomal aminopeptidase, CD13, p150), APN, LAP1, PEPN, gp150, aminopeptidase M, aminopeptidase N, membrane alanine aminopeptidase, and microsomal aminopeptidase. The term CD14 includes CD14 and any orthologs thereof.

The term CD29 includes CD29 and any orthologs thereof, including but not limited to ITGB1, integrin, beta 1 fibronectin receptor, beta polypeptide, MDF2, MSK12, FNRB, GPIIA, MDF2, MSK12, VLAB, OTTHUMP00000046253, OTTHUMP00000063731, OTTHUMP00000063732; OTTHUMP00000063733, fibronectin receptor beta subunit, integrin VLA-4 beta subunit, and integrin beta 1.

The term CD31 includes CD31 and any orthologs thereof, including but not limited to PECAM1, platelet/endothelial cell adhesion molecule, CD31/EndoCAM, PECAM-1, and CD31/EndoCAM adhesion molecule.

The term CD33 includes CD33 and any orthologs thereof, including but not limited to FLJ00391, SIGLEC-3, SIGLEC3, p67.

The term CD36 includes CD36 and any orthologs thereof, including but not limited to DKEY-27K7.2, zgc:92513, and fatty acid translocase.

The term CD28 includes CD28 and any orthologs thereof, including but not limited to ADP-ribosyl cyclise, and cyclic ADP-ribose hydrolase.

The term CD49f includes CD49f and any orthologs thereof, including but not limited to Itga6, integrin alpha 6, RP23-5K9.4, 5033401O05Rik, AI115430, and Cd49f.

The term CD62 includes CD62 and any orthologs thereof, including but not limited to SELP, selectin P, granule membrane protein 140 kDa, CD62P, FLJ45155, GMP140, GRMP, LECAM3, PADGEM, PSEL, granulocyte membrane protein, leukocyte-endothelial cell adhesion molecule 3, and platelet alpha-granule membrane protein.

The term CD73 includes CD73 and any orthologs thereof, including but not limited to NT5E, 5′-nucleotidase, ecto, RP11-321N4.1, E5NT, NT, NT5, NTE, eN, eNT, 5′ nucleotidase (CD73), 5′ nucleotidase, OTTHUMP00000040565, Purine 5-Prime-Nucleotidase, and ecto-5′-nucleotidase.

The term CD105 includes CD105 and any orthologs thereof, including but not limited to ENG, endoglin, RP11-228B15.2, END, FLJ41744, HHT1, ORW, and ORW1.

The term CD106 includes CD106 and any orthologs thereof, including but not limited to VCAM1, vascular cell adhesion molecule 1, DKFZp779G2333, INCAM-100, and MGC99561.

However, the cell population in some embodiments of the invention is characterised in that the cells express one or more of the markers Cd11b, CD13, CD14, CD29, CD31, CD33, CD36, CD38, CD49f, CD62, CD73, CD105, and CD106 at a level that is lower than the level of their expression in embryonic stem cells. The comparison between the expression level of the markers in the adult stem cell of the invention, and the expression level of the markers in an embryonic stem cell may be conducted with an adult stem cell and an embryonic stem cell isolated from the same species. Preferably this species is a mammal, and more preferably this species is human. Such a comparison may be conducted using a reverse transcriptase polymerase chain reaction (RT-PCR) experiment.

In a further embodiment, the adult stem cell population may also express markers which show the commitment of the cells of the invention to a specific tissue. In particular, when the cells of the invention are isolated from cardiac tissue and, in some embodiments after long term propagation, they may express one or more of the following markers MEF2C, GATA-4, ANF, MCL2a, MCL2v, Msi-1, P75, Pax3, P0, Nestin or NRx2.5. Cells displaying such markers may be obtained after more than 80 population doublings, more than 90 population doublings, more than 100 population doublings, more than 120 population doublings or more than 130 population doublings.

The markers described above are considered not to be expressed by a cell population of the invention, if expression cannot be reasonably detected at a level of about 10-20 copies per cell.

In a further embodiment, the adult stem cell population expresses telomerase. In some embodiments, the stem cell population of the invention is considered to express telomerase if at least about 70% of the cells of the isolated adult stem cell population show detectable expression of telomerase. In other embodiments, at least about 80%, at least about 90% or at least about 95% or at least about 97% or at least about 98% or at least about 99% or 100% of the cells of the stem cell population show detectable telomerase expression.

Telomerase is considered to be expressed by a cell population of the invention, if expression can be reasonably detected at the RNA level following 30 cycles of PCR, and by the in vitro “telomerase reaction” at the protein level, which corresponds to an expression level in the cell of less than about 1/10 of the enzyme activity detected in a HeLa cell, which corresponds to a level of less than about 100 molecules per cell. The normal level of telomerase expression of a single cell of the invention is higher than 100-10,000 normal somatic cells

In some embodiments, the adult stem cell population will show at least about 100-10,000 times more telomerase expression than a somatic adult stem cell. In other embodiments, the adult stem cell population will show at least about 100 times, at least about 1000 times, at least about 10,000 or at least about 100,000 more telomerase expression than a somatic adult cell. This telomerase expression may be stable and may be maintained through multiples passages. In some embodiments, the telomerase expression may be maintained through 10 passages, through 50 passages, through 100 passages, through 200 passages, through 300 passages or more. In some embodiments the telomerase expression may be maintained by the cloned and/or the subcloned cells. The comparison between the adult stem cell population and the somatic adult cell may be performed using cells isolated from individuals of the same species. Preferably, the cells are isolated from a mammal, and more preferably the cells are isolated from a human. In some embodiments the cells are isolated from the same individual. Such analysis may be performed using an RT-PCR experiment. It will be clear to a person skilled in the art that this method is provided by way of illustration only, and that other detection methods known in the art may also be used.

Cellular Morphology

The cellular morphology of the cells of the invention is an important aspect of the invention. Unlike the adult stem cells that have previously been isolated from non-neonate tissue, the isolated adult stem cells of the invention have cellular morphology which resembles a totipotent embryonic stem cell. The cells of the invention are of a very small size, for example about 5 μm in diameter. The cells have a large nucleus with loose chromatin surrounded by a rim of cytoplasm. When placed in culture, the cells of the invention attach very slowly to the cell culture dish and may remain in suspension for more than 24 hours, and in some embodiments up to 72 hours, a property which can be exploited for their enrichment. In vivo, the cells of the invention are located individually in the interstitial left by the differentiated cells of tissue, and are often surrounded by other small c-kit positive cells that have already lost the expression of Oct-4, Nanog and SSEA-1 in the murine cells and SSEA4 in the human cells. The latter cells may be the progeny of the cells of the invention, which, until recently had been considered to be the true cardiac stem cells (Beltrami et al., 2003. Cell 114: 763-776.) Interestingly, the inventors have discovered that when there are several lineage negative, c-kit positive cells in an interstitium of a tissue, there is only one cell among them that is positive for Oct4 or any of the other multipotency gene markers, strongly suggesting that this is the true adult tissue stem cell while the other c-kit positive cells in the vicinity are its progeny and represent progenitors and/or precursors for the different tissue-specific cell types (FIG. 17). In normal tissues almost all the cells of the invention are quiescent as determined being negative for Ki-67 expression, lack of uptake of BrdU except in very long labelling pulses and once labelled retaining the label for a very long time (up to several months). All these characteristics are those expected for true tissue stem cells.

Cellular Functionality

A major characteristic of the isolated adult stem cells of the present application is their ability to both self-renew and to produce progeny committed to the differentiation pathway. As discussed above, cells that have previously been considered adult stem cells in the art are in fact considered by the inventors to be the progeny of the cells of the invention and to have already committed to the differentiation pathway and restricted their developmental potential, generally to the production of parenchymal cells of the tissue of origin. On the contrary, the adult stem cells of the invention thus have the capacity to maintain the pluripotent capacity as well as the capacity to produce progeny committed to the differentiation pathway. Also within this characteristic, the adult stem cell of the present invention has the capacity to produce progeny which will differentiate into partially or full differentiated cells. The capacity of the adult stem cell of the invention to produce differentiated of its progeny can occur in vivo or in vitro. In certain aspects, the isolated adult stem cell population is considered to be capable of controlling the differentiation capacity of its progeny if at least about 70% of the cells of the isolated adult stem cell population are capable of producing differentiated progeny. In some embodiments, at least about 80%, at least about 90% or at least about 95%, 99% or more of the cells of the isolated adult stem cell population are capable of differentiating.

This differentiation is controlled, at least in part, by exogenous factors added to the culture medium or secreted by the surrounding tissue cells and also through a paracrine effect initiated by the cells of the invention over their progeny cells, in particular surrounding progeny cells. This paracrine effect is caused by the secretory activity of the cells of the invention, which is high in cell growth factors, multiple different cytokines and chemokines that are capable of stimulating or inhibiting the growth, differentiation and locomotion of the progeny cells. As shown in FIG. 19, in some embodiments the cells of the invention differentiate into beating cardiac myocytes with well formed sarcomeres and which assemble into functional syncitia through gap junctions containing connexin 43, as in the myocardium.

The adult stem cell population of the invention is also capable of forming pseudo embryoid bodies. In certain embodiments, the isolated adult stem cell population is considered to be capable of forming pseudo embryoid bodies if at least about 70% of the cells of the isolated adult stem cell population are capable of forming pseudo embryoid bodies. In some embodiments, at least about 80%, at least about 90% or at least about 95%, 99% or more of the cells of the isolated adult stem cell population are capable of forming embryoid bodies. Conventionally, such pseudo embryoid bodies are produced by the hanging drop method. However, the cells of the invention form embryoid bodies readily when cultured in growth medium in bacterial culture dishes which are not coated with negative charge. It will be clear to a person skilled in the art that this definition is not intended to be limiting, and that any method known in the art for the production of embryoid bodies may be utilised. The formation of pseudo embryoid bodies is required to allow the cells to differentiate into spontaneously beating cardiac myocytes when allowed to attach to a culture dish in the presence of the appropriate differentiation medium. The ability of the cells of the invention to form pseudo embryoid bodies when plated at low density in bacterial dishes is a characteristic of their ability to self-renew which can be exploited for their isolation and separation from the progenitors and precursors from the same tissue which are still positive for the expression of c-kit. Under low density cultures or when plated as single cells in Terasaki plates, only the cells expressing the multipotency genes (i.e. the cells of this invention) are capable of forming pseudo embryoid bodies, while the progenitors and precursors derived from them are not. The progenitors and precursors are only able to participate in the formation of pseudo embryoid bodies when plated at high density when the pseudo embryoid bodies are formed by cell aggregation and not by clonal expansion of a single cell.

The adult stem cell population is also capable of self-renewing. That is, the cells of the invention give rise to daughter cells with the same characteristics and development potential (tripotency) as the mother cell. This characteristic can be determined through the capacity of a culture of the cells on the invention to undergo multiple passages without loosing its tripotency. Human, mouse, rat and pig cells of the invention have been passed for more than 100 passages without a loss of the tripotency. A more stringent assay of self-renewal is by testing the characteristic of single cell clones and clones derived from these clones. As shown in FIG. 20, the cloning frequency of the cells of the invention is extremely high for primary cells of any type (−15%). Up to three rounds of cloning for the cells of each species have shown the stability of robustness of the self-renewal capability of these cells. More impressive, starting from a single cell, we have expanded the culture it up to 5×10¹¹ cells without changes in karyotype, cell surface markers and developmental potential. Consequently, in certain aspects of the invention, the isolated adult stem cell population is considered to be capable of self-renewing if at least about 70% of the cells of the isolated adult stem cell population are capable of self-renewing. In some embodiments, at least about 80%, at least about 90% or at least about 95%, 99% or more of the cells of the isolated adult stem cell population are capable of self-renewing, when cultivated in “growth medium” as described below, preferably in mass cell cultures. If tested by the cloning assay described in Example 3, where the cells of the invention are plated at 0.5 cells per well in Terasaki plates in some embodiments up to 95% of the clones are identical to the starting cell population. In some embodiments at least about 80%, at least about 90% or at least about 95%, 99% or more of the clones are identical to the starting cell population. To maintain the self-renewing property of the cells, the cells may need to be grown in growth medium or in cloning medium which comprises a mixture of “conditioned medium” and “growth medium”. In some embodiments, said cloning medium contains at least about 20% of “conditioned medium”, at least about 30%, at least about 40%, at least about 50% or more of “conditioned medium”. In some embodiments, said cloning medium contains at least about 20% of “growth medium”, at least about 30%, at least about 40%, or at least about 50% or more of “growth medium”.

The cloning medium may comprise about 50% “growth medium” and about 50% “conditioned medium”.

The growth medium may be Medium I, II, III or IV.

The growth media may be medium I, which comprises 10% embryonic stem cell-qualified fetal bovine serum (ES-FBS, Invitrogen); 10 ng/ml mouse basic fibroblast growth factor (FGFb, PeproTech), 20 ng/ml mouse endothelial growth factor (EGF, PeproTech), 10 ng/ml mouse leukaemia inhibitory factor (LIF, Chemicon); 6.7 ng/ml sodium selenite, 10 μg/ml insulin, 5.5 μg/ml transferring, 2 μg/ml ethanolamine (ITS, Invitrogen); 50 μg/ml gentamycin, 0.1 mg/ml streptomycin and 100 U/ml penicillin (Sigma); 250 ng/ml amphotericin B, and 205 ng/ml sodium deoxycholate (Fungizone, Invitrogen) in 1:1 Dulbecco's Modified Eagle's Medium/Nutrient Mixture F-12 Ham (DMEM/F12, Sigma).

The growth media may be medium II, which is the same as Medium I, but wherein the serum was depleted of differentiation factors and other high molecular weight proteins by treatment with DCC solution, prepared as followed: 0.45 g of dextran T500 and 4.5 g activated charcoal (Sigma) were stirred overnight at 4° C. in 1800 ml 0.01M Tris-HCl (Sigma), pH 8.0 in a tightly closed Erlenmeyer bottle. DCC solution was centrifuged at 2000 g for 20 min in 50 ml plastic tubes, the supernatant was discarded and new DCC solution was added to the same tubes and centrifuged again, in order to obtain “double pellets”. After inactivating the FBS 30 min at 56° C., 50 ml of FBS was mixed with each double pellet and transferred to a glass bottle, incubating the mixture for 45 min at 45° C. under shaking. Afterwards, the mixture was centrifuged 20 min at 2000 g and the supernatant was mixed with a new DCC double pellet and incubated again 45 min at 45° C. in a glass bottle under shaking. After centrifuging 20 min at 2000 g, the FBS supernatant was sterilized through a 0.22 μm low protein binding filter.

The growth media may be medium III which comprises 10 ng/ml mouse basic fibroblast growth factor (FGFb, PeproTech), 10 ng/ml mouse endothelial growth factor (EGF, PeproTech), 10 ng/ml mouse leukaemia inhibitory factor (LIF, Chemicon); 0.1 mM 2-mercaptoethanol, 1 mM L-glutamate, 15 nM sodium selenite, 25 μg/ml BSA (Sigma); 0.5× Bottenstein's N-2 supplement, 0.5×B27 supplement without vitamin A (Invitrogen); 50 μg/ml gentamycin, 0.1 mg/ml streptomycin and 100 U/ml penicillin (Sigma); 250 ng/ml amphotericin B, and 205 ng/ml sodium deoxycholate (Fungizone, Invitrogen) in 1:1 Neurobasal (Invitrogen) and DMEM/F12 (Sigma) media.

The growth media may be medium IV, which comprises 10% embryonic stem cell-qualified fetal bovine serum (ES-FBS), 5% horse serum (Invitrogen); 10 ng/ml mouse basic fibroblast growth factor (FGFb, PeproTech), 20 ng/ml mouse endothelial growth factor (EGF, PeproTech), 10 ng/ml mouse leukaemia inhibitory factor (LIF, Chemicon); 5 mU/ml erythropoietin, 50 μg/ml porcine gelatin, 0.2 mM L-glutathione, 50 μg/ml gentamycin, 0.1 mg/ml streptomycin and 100 U/ml penicillin (Sigma); 250 ng/ml amphotericin B, and 205 ng/ml sodium deoxycholate (Fungizone, Invitrogen) in F-12K nutrient mixture with Kaighn's modification (Invitrogen), pH 7.4.

The “conditioned medium” may be a growth medium for stem cells, which has been used to feed a mass culture of stems cells, embryonic stem cells or cells of the invention for at least about 12 hours, at least about 24 hours, at least about 48 hours or least about 72 hours, removed and sterilized by any suitable mean, preferably by filtration, prior to use, if required.

In some embodiments, the isolated adult stem cell population does not show gap junction intercellular communication (GJIC). In certain aspects, the isolated adult stem cell population is considered not to show GJIC if at least about 70% of the cells of the isolated adult stem cell population do not show GJIC. In some embodiments, at least about 80%, at least about 90% or at least about 95%, 99% or more of the cells of the isolated adult stem cell population do not show GJIC. In certain aspects, the isolated adult stem cell population is considered not to show GJIC if the level of CJIC is at least about 70% less than the level of GJIC shown by an adult somatic cell. In other embodiments, the adult stem cell population will show at least about 80% less than the level of GJIC shown by an adult somatic cell, at least about 90% less, at least about 95% less, at least about 97% less, at least about 98% less, at least about 99% less or 100% less than the level of GJIC shown by an adult somatic cell. The comparison between the adult stem cell population and the somatic adult cell may be performed using cells isolated from the same species. Preferably this species is a mammal, and more preferably this species is a human. In some embodiments, the cells may be isolated from the same individual. GJIC may be measured using fluorescent dye transfer measurement. It will be clear to a person skilled in the art that this method if provided by way of illustration only, and that other detection methods known in the art may also be used. However, the cells of the invention, when induced to differentiate into cardiac tissue, using DMI or DMII supplemented with differentiation one or more of Wnt5a, TGFβ-1, BMP-4 and BMP-2, develop anatomical and functional gap junctions containing connexion 43 (GJ43). In certain aspects, the isolated adult stem cell population is considered to develop anatomical and functional gap junctions containing connexion if at least about 70% of the cells of the isolated adult stem cell population develop GJ43, when they are induced to differentiate into cardiac tissue. In some embodiments, at least about 80%, at least about 90% or at least about 95%, 99% or more of the cells of the isolated adult stem cell population develop GJ43, when they are induced to differentiate into cardiac tissue.

Immunogenicity

The adult stem cells of the invention either do not trigger an immune response in vitro or in vivo or trigger an immune response which is substantially weaker than that which would be expected to be triggered upon injection of a cell population into a patient. In certain aspects of the invention, the adult stem cell population is considered not to trigger an immune response if at least about 70% of the cells of the isolated adult stem cell population do not trigger an immune response. In some embodiments, at least about 80%, at least about 90% or at least about 95%, 99% or more of the cells of the isolated adult stem cell population do not trigger an immune response. Preferably the cells of the invention do not trigger an antibody mediated immune response or do not trigger a humoral immune response. When allogeneic cells of the invention are administered by the intracardiac, intravenous or subcutaneous route to allogeneic pigs, rats and mice no allo-antibodies can be detected in the host up to 2 month later (data not shown). More preferably the cells of the invention do not trigger either an antibody mediated response or a humoral immune response in vitro. More preferably still, the cells of the invention do not trigger a mixed lymphocyte immune response. It will be understood by one skilled in the art that the ability of the cells of the invention to trigger an immune response can be tested in a variety of ways. By way of illustration only, it is possible to establish whether the cells of the invention trigger an immune response by culturing the cells of the invention with T-cells from a non-matched individual. In order for cells to be capable of inducing an immune response, the culturing of such cells will result in stimulation of the proliferation of the T-cells. Assays for testing this capability are well known to the skilled reader. By way of illustration only, an exemplary assay for detecting whether cells of the invention elicit an immune response may involve incubating the isolated adult stem cells with T cells from an unmatched individual, and measuring the consequent immune response relative to the immune response elicited with terminally differentiated cells isolated from both a matched and an unmatched individual. It will be apparent to one skilled in the art that the consequent immune response may be measured in a number of different ways. Firstly, the extent of the immune response may be measured by detecting the level of one or more of a number of cytokines associated with production of an immune response. In one embodiment, the immune response may be measured by detecting the levels of one or more cytokines. Cytokines suitable for detection of an immune response may include IL2, IFNλ, TNFβ, IL4, IL5, IL10, IL3, TNFα and TGFβ. It will be clear to one skilled in the art that the levels of one or more of these cytokines may detected in order to measure the extent of the immune response.

In one embodiment, the cells of the invention will be considered not to trigger an immune response if the level of expression of one or more of IL2, IFNλ, TNFβ, IL4, IL5, IL 10, IL3, TNFα and TGFβ, induced following incubation of the isolated adult stem cells with T cells from an unmatched individual, is less than about 50% of the level of expression of one or more of IL2, IFNλ, TNFβ, IL4, IL5, IL10, IL3, TNFα and TGFβ following incubation of an equivalent T cell population with terminally differentiated cells isolated from a non-matched individual. In some embodiments, the level of expression of one or more of IL2, IFNλ, TNFβ, IL4, IL5, IL10, IL3, TNFα and TGFβ induced following incubation of the isolated adult stem cells with T cells from an unmatched individual is less than about at 40%, about 30%, about 25%, about 20% or about 10% or less of the level of expression of one or more of IL2, IFNλ, TNFβ, IL4, IL5, IL10, IL3, TNFα and TGFβ following incubation of a equivalent T cell population with terminally differentiated cells isolated from a non-matched individual.

In another embodiment, the cells of the invention will be considered not to trigger an immune response if the level of expression of one or more of IL2, IFNλ, TNFβ, IL4, IL5, IL10, IL3, TNFα and TGFβ, induced following incubation of the isolated adult stem cells with T cells from an unmatched individual, is more than about 50% of the level of expression of one or more of IL2, IFNλ, TNFβ, IL4, IL5, IL10, IL3, TNFα and TGFβ following incubation of an equivalent T cell population with terminally differentiated cells isolated from a matched individual. In some embodiments, the level of expression of one or more of IL2, IFNλ, TNFβ, IL4, IL5, IL10, IL3, TNFα and TGFβ induced following incubation of the isolated adult stem cells with T cells from an unmatched individual is less than about at 60%, about 70%, about 75%, about 80% or about 90% of the level of expression of one or more of IL2, IFNλ, TNFβ, IL4, IL5, IL10, IL3, TNFα and TGFβ following incubation of a equivalent T cell population with terminally differentiated cells isolated from a matched individual.

In another embodiment, the consequent immune response produced by the assay described above may be measured by detecting the doubling rate of the T cells in the assay. In one embodiment, the cells of the invention will be considered not to trigger an immune response if the doubling rate of T cells from an unmatched individual, following incubation with the isolated adult stem cells, is less than about 50% of the doubling rate of an equivalent population of T following incubation with terminally differentiated cells isolated from a non-matched individual. In some embodiments, the doubling rate induced following incubation of the isolated adult stem cells with T cells from an unmatched individual is less than about at 40%, about 30%, about 25%, about 20% or about 10% or less of the T cell doubling rate following incubation of an equivalent T cell population with terminally differentiated cells isolated from a non-matched individual.

In another embodiment, the cells of the invention will be considered not to trigger an immune response if the doubling rate of TNF-β-stimulated T cells from an unmatched individual, following incubation with the isolated adult stem cells, is more than about 50% of the doubling rate of an equivalent population of T following incubation with terminally differentiated cells isolated from a matched individual. In some embodiments, the T cell doubling rate induced following incubation of the isolated adult stem cells with T cells from an unmatched individual is less than about at 60%, about 70%, about 75%, about 80% or about 90% or more of the doubling rate following incubation of a equivalent T cell population with terminally differentiated cells isolated from a matched individual.

The assays described above are provided by way of illustration only, and are not intended to be exhaustive. The skilled person will be aware of various alternative assays which might be used.

In the mix lymphocyte reactions (MLR) described herein, where human, mouse and pig cells have been tested, mesenchymal (MSCs) and embryonic (ESCs) stem cells have been used for comparison. In all tests, the cells of the invention have shown a stronger immunosuppressive activity than the MSCs and comparable immunosuppressive activity to the ESCs.

Major histocompatibility complexes (MHC) are complexes of glycoproteins which are expressed on a cell's surface and present peptide antigens to the immune system. There are two forms of MHCs, class I (MHC I) and class II (MHC II). MHC I are present on the surface of virtually all cells; they bind peptide antigens generated via cytosolic protein degradation pathway and present these antigens to CD8⁺ T cells. MHC II are present on the surface of professional antigen presenting cells; they bind peptide antigens generated via the endocytic pathway, and present these antigens to CD4⁺ T cells. To date, the only cells known to express neither MHC I nor MHC II are erythrocytes.

Within a further aspect of the invention, the isolated adult stem cells express major histocompatibility complex I (MHC I) at a low level. MHC I is considered to be expressed at a low level if the level of expression is less than about 1/10 of the level of expression in a differentiated cell. This value is provided by way of examples only, and is not intended to be limiting. It is therefore possible that a small amount of residual expression persists but not enough to confer any immune function. In certain aspects of the invention, the adult stem cell population of the invention is considered not to express MHC I if at least about 70% of the cells of the isolated adult stem cell population do not express MHC I. In some embodiments, at least about 80%, at least about 90% or at least about 95%, 99% or more of the cells of the isolated adult stem cell population do not express MHC I. When analysed by immunofluorescence on fixed cells, the level of MHC I expression by the cells of the invention is very heterogeneous. A possible explanation for this is that the heterogeneous expression of MHC I molecules is a reflection of slightly different stages of differentiation of the cells, with those progeny which are more advanced along the differentiation pathway expressing higher levels of MHC I.

Within a further aspect of the invention the adult stem cells of the invention do not express major histocompatibility complex II (MHC II). MHC II is considered not to be expressed by a cell of the invention if expression cannot be reasonably detected at a level of about 1/50 of the level of expression in a differentiated cell. It is therefore possible that a small amount of residual expression persists but not enough to confer any immune function. In certain aspects of the invention, the isolated adult stem cell population is considered not to express MHC II if at least about 70% of the cells of the isolated adult stem cell population do not express MHC II. In some embodiments, at least about 80%, at least about 90% or at least about 95%, 99% or more of the cells of the isolated adult stem cell population do not express MHC II.

In a further embodiment of the invention, the isolated adult stem cells express neither MCH I or MHC II. The definitions of “expressed” are intended to be the same as those given above for MHC I and MHC II respectively. As described previously, this is a remarkable finding since previously, the only cells thought to express neither MIIC I nor MHC II were erthyrocytes.

It will be understood by a person skilled in the art, that the assay described above can be adapted in order to determine the expression levels of MHC I and MHC II respectively. Expression of certain cytokines is known to be induced specifically by activation of T cells through MHC I or MHC II respectively. For example, it is well known in the art that expression of IL2, IFNλ and TNFβ is induced by T cell activation though MHC I. It is also well known in the art that expression of IL4, IL5 and IL10 if induced by T cell activation through MHC II, and that expression of IL3, TNFα and TGFβ is induced by T cell activation through either MHC I or MHC II. It will therefore be evident to one skilled in the art that the expression levels of these cytokines, relative to the expression levels in terminally differentiated cells isolated from both a matched and an unmatched individual when each cell population is separately incubated with T cells from an unmatched individual, will indicate the expression of MHC I and MHC II respectively.

A further characteristic of the adult stem cells of the present invention is their ability not to induce formation of a tumor upon injection of the isolated stem cell of the invention into a host organism. In one aspect of the invention, this tumor is a teratoma. In certain aspects of the invention, the isolated adult stem cell population is considered not to induce formation of a teratoma upon injection into a host if at least about 70% of the cells of the isolated adult stem cell population do not induce production of a teratoma upon subcutaneous or intramuscular injection into a syngeneic and/or immunodeficient host. In some embodiments, at least about 80%, at least about 90% or at least about 95%, 99% or more of the cells of the isolated adult stem cell population do not induce formation of a teratoma upon injection into a host. Administration up to 5×10⁷ cells of the invention into syngeneic rodents and pigs did not produced any detectable teratomas. Administration of 5×10⁶ human cells into immunodeficient mice, intramuscularly and subcutaneously, also did not produce any detectable teratomas (data not shown).

Furthermore, in a further aspect the cells of the invention are not capable to form tumors when injected into the systemic circulation of syngeneic animals and/or immunodeficient mice at a dose of 1×10⁶ cells per animal (maximal tolerated dose for the mouse).

Adult Stem Cell Isolation

The adult stem cells of the invention can be isolated from any non-embryonic tissue. Preferably the tissue is a mammalian tissue, and more preferably the tissue is a human tissue. In certain aspects, the tissue may be obtained from an individual of between 18 and 70 years of age. Preferably, the individual should be of between 18 and 50 years of age in order to obtain cells with robust growth and differentiation properties. Data from humans ranging from 5 up to 85 years of age and mice and rats from birth up to two years show that the cells of the invention are more abundant in young animals and their frequency diminish with age (see FIG. 3, bottom panel). The cells of the invention can be up to 10-20 fold more abundant in a post-puberal/young subject than in a mature/old one.

It is thought that the cells can be isolated from any tissue found in an adult subject. Within one aspect of the invention, the adult stem cells of the invention may be isolated from cardiac tissue. In certain aspects, the isolated adult stem cells of the invention are isolated from non-neonate myocardium. In some embodiments, the isolated adult stem cells of the invention may be isolated from non-neonate atrial or ventricular myocardial walls or interatrial and interventricular septum. The cells can be isolated from the hearts of sacrificed animals, from small cardiac human biopsies obtained during cardiac surgery, or by means of a biopsy catheter during cardiac catheterism. They can also be obtained from hearts harvested for cardiac transplant and also from the excised hearts of recipients of heart transplants.

Within a further aspect of the invention, the adult stem cells may be isolated from side population from cells isolated from the heart, the bone marrow, skeletal muscle and brain. These cells have been isolated from different species such as mouse, rat, pig and human, therefore it is assumed that they are present in all mammals. Preferably, said side population cells express the MDR1 gene and can be detected by their exclusion of the dye Hoechst 33343 when the multidrug transporter is blocked (FIG. 12).

As will be clear to one of skill in the art, the present invention concerns an isolated adult stem cell population per se, and uses thereof. The particular method used for isolation of the cells is not an essential feature of the invention. Nevertheless, with the knowledge of the existence of the stem cell population of the invention, and the characteristic features thereof as detailed above, a number of methods are known in the art which can be utilised to isolate these cells from non-embryonic tissue, and these are described below. This method is provided by way of illustration only, and is not intended to be limiting. In one embodiment, a method of isolating the adult stem cells of the invention may comprise: (a) collecting tissue from a subject; (b) obtaining a cell suspension by either enzymatic digestion or other means of tissue dissociation; (c) sedimenting the cell suspension and resuspending the cells in a culture medium; (d) separating the smaller cells from the majority of the parenchymal cells of the tissue by differential centrifugation; e) removing the so called “lineage positive cells” from the mixture by means of a specific antibody cocktail; f) conjugating the “lineage negative cells” to an antibody specific for a membrane marker diagnostic of the cells on the invention, in this case a species-specific anti c-kit antibody; g) isolating the c-kit positive, Sca 1 negative cells by means of a second antibody either through a immuno-column or by means of immunobeads; h) plating single cells in Tesaki plates at a density of ½ cell/well; i) culturing of the cells for at least about 10 days or after the growth of clones; (j) expanding the cells for at least two culture passages; k) screening the clones for the expression of the multipotency genes; and l) expanding the positive clones for further characterization.

The above protocol can be modified after step e) and the “side population” isolated by cell sorter, followed by proceeding to step h) as above (See FIG. 12).

To further enrich the desired cell population prior to the cloning step is to isolate the cells that are double positive: for c-kit and SSEA1 or SSEA4 depending whether the tissue of origin is rodent or human.

It is also possible to identify the cells of the invention by plating the cells after step g) in mass culture with growth medium supplemented with only 1% of FCS.

After two weeks of culture, in any one of media I-IV described above, clones of rounded cells expressing the multipotency genes appear in the culture (FIGS. 10 and 21). These cells can be physically isolated and expanded with characteristics similar to those isolated by the other protocols described above.

An additional method of isolating the cells of the invention is to plate the cells after step f) or g) at clonal density in bacteriological culture plates to stimulate the formation of pseudo-embryoid bodies in DMI or DMII. Only the cells of the invention form clonal pseudo-embryoid bodies which can be isolated and their cells subcloned to insure purity and expanded. In another embodiment the cells of the invention can be isolated using a reporter vector expressing either a fluorescent protein (eg EGFP, YGFP, etc) or a selectable marker (such as puromycin resistance). The dissociated cells either before or after the selection of the c-kit positive cells are transfected with a lentivirus construct carrying the proper marker driven by one of the four main multipotency gene promoters (Oct4, Sox2, Nanog and Klf4). The cells of the invention can be isolated by fluorescence activated cell sorting after a few days or after all the drug sensitive cells have been eliminated if the selection is by drug resistance.

This isolation method may use reporters driving the expression of a fluorescent protein or a drug selectable marker under the control of one of the major multipotency gene promoters. Once the c-kit^{pos} cells have been removed from the small cell population either by sorting or with the use of immunobeads, the remainder of the cells may be transfected with the corresponding construct and after 1-2 weeks either the fluorescent cells are sorted or the culture is treated with the corresponding cytotoxic drug to kill all the cells which fail to express the drug resistance marker. Over 85% of the drug resistant clones originated in this manner, are composed of cells expressing the endogenous multipotency genes. This approach can be used for the cells from different tissues and species from mouse to man.

From species where it is possible to obtain a whole organ with intact circulatory system such as mouse and rats, the most efficient method to obtain dissociated cells is by retrograde perfusion with a proteolytic solution. When retrograde perfusion is not possible two other approaches can be used:

Any one of a number of physical methods of separation known in the art may be used to select the cells of the invention and distinguish these from other cell types. Such physical methods may involve FACS and various immuno-affinity methods based upon makers specifically expressed by the cells of the invention. As described above, c-kit, Nanog, SSEA1 and Oct-4 are 3 of the cell markers expressed at high levels in the cells of the invention. Therefore, by way of illustration only, the cells of the invention may be isolated by a number of physical methods of separation, which rely on the presence of these markers.

In one embodiment, the cells of the invention may be isolated by FACS utilizing an anti-c-kit antibody. As will be apparent to one skilled in the art, this may be achieved through a fluorescent labeled anti-c-kit antibody, or through a fluorescent labeled secondary antibody with binding specificity for the anti-c-kit antibody. Examples of suitable fluorescent labels includes, but is not limited to, FITC, Alexa Fluor® 488, GFP, CFSE, CFDA-SE, DyLight 488, PE, PerCP, PE-Alexa Fluor® 700, PE-Cy5 (TRI-COLOR®), PE-Cy5.5, PI, PE-Alexa Fluor® 750, and PE-Cy7. This list is provided by way of example only, and is not intended to be limiting.

It will be apparent to a person skilled in the art that FACS analysis using an anti-c-kit antibody will provide a purified cell population. However, in some embodiments, it may be preferable to purify the cell population further by performing a further round of FACS analysis using one or more of the other identifiable markers, preferably Nanog, SSEA1 or Oct-4, but others listed above may also be used.

In another embodiment, the cells of the invention may be isolated by immuno-affinity purification, which is a separation method well known in the art. By way of illustration only, the cells of the invention may be isolated by immuno-affinity purification directed towards c-kit. As will be apparent to one skilled in the art, this method relies upon the immobilisation of anti-c-kit antibodies on a purification column. The cell sample is then loaded onto the column, allowing the appropriate cells to be bound by the anti-c-kit antibodies, and therefore bound to the column. Following a washing step, the cells are eluted from the column using a competitor which binds preferentially to the immobilised anti-c-kit antibody, and permits the cells to be released from the column.

It will be apparent to a person skilled in the art that immuno-affinity purification using an immobilised anti-c-kit antibody will provide a purified cell population. However, in some embodiments, it may be preferable to further purify the cell population by performing a further round of immuno-affinity purification using one or more of the other identifiable markers, for example SSEA-1, and use an aliquot of the isolated clones to ascertain the expression of the intracellular markers such as Nanog, Oct4, Sox, etc.

It will be apparent to a person skilled in the art that the sequential purification steps are not necessarily required to involve the same physical method of separation. Therefore, it will be clear that, for example, the cells may be purified through a FACS step using an anti-c-kit antibody, followed by an immuno-affinity purification step using a SSEA-1 affinity column. In certain embodiments, the cells may be cultured after isolation for at least about 15, at least about 20 days, at least about 25 days, or at least about 30 days. In certain aspects, the cells are expanded in culture longer to improve the homogeneity of the cell phenotype in the cell population.

In one embodiment, a tissue sample from a donor (usually comprising between about 50 and 150 mgs of tissue) is finely minced with razor blades on a tissue dish in a drop of growth medium.

When the particles of tissue are smaller than about 1 mm³ they are placed at least 1 cm apart at the bottom of the dish and individually covered with a glass porta. The dish is filled with growth medium to a height of about 3 mm and incubated in a cell incubator for between about 7 and 10 days. At a rate which is usually inversely correlated with the age of the donor, the tissue explants grow a halo of cells which sprout out of the tissue. When the halo contains a few thousands cells, the remaining carcass of the tissue sample is removed, and the cells are trypsinised, and transferred into a large well. These cells are expanded until there are between about 2×10⁶ and 3×10⁶ cells. The cells are harvested and enriched for the cells of the invention by passing them through a column of beads with anti c-kit antibodies attached to their surface (Miltenyi Biotech). Once the c-kit negative cells have been eluted and discarded, the cells attached to the column are released and plated in growth medium. When the cells have recovered and expanded to between 1×10⁶ and 2×10⁶ cells, they are harvested again and further purified by cell sorting using a fluorescently tagged anti c-kit antibody. The c-kit positive cells are placed in individual wells and allowed to grow as clones. Aliquots of the clones are tested for expression of Oct-4 and/or Nanog. The positive clones are further analyzed for the characteristics described above. The clones that express the appropriate phenotype are chosen for further growth, analysis and use. Aliquots of each clone are stored frozen after every 5 passages.

In another embodiment, the tissue sample is digested with proteolytic enzymes, either in a test tube or, if it is the whole heart e.g. mouse or rat, by retrograde perfusion through the cannulated aorta. When many of the cells of the tissue have become loose, they are separated by size either by running them through a size exclusion column or by differential centrifugation. The large cells, which are usually mainly myocytes are discarded, and the very small cells are passed through the anti c-kit Miltenyi column to obtain the population of c-kit positive cells which contain the cells of the invention at about between 3 and 5% purity. Also contained within this cell population are the most abundant precursors, progenitors and contaminating small differentiated cells. Subsequently the c-kit positive cells are processed as described above.

The purity of the isolates can be enhanced by using, in series, two different Miltenyi columns, one anti c-kit followed by and anti SSEA1. However, this method of purification may result in a decrease in the viability and clonability of the cells obtained. Starting with a tissue sample, in the hand of an experienced investigator, it takes between about 5 and 7 weeks, in some embodiments about 3 to 5 weeks, to obtain the cells of the invention in cloned form.

In certain embodiments, the isolated cells are expanded in culture for at least three culture passages. In other embodiments, the cells are passaged at least four times, at least five times, at least six times, at least seven times, at least eight times, at least nine times, or at least ten times or more. Again, the cells may be passaged more than three times to improve the homogeneity of the cell type in the cell population. Indeed, the cells may be expanded in culture indefinitely so long as the homogeneity of the cell phenotype is improved and differential capacity is maintained.

Cells may be cultured by any technique known in the art for the culturing of stem cells. A discussion of various culture techniques, as well as their scale-up, may be found in Freshney, R. I., Culture of Animal Cells: A Manual of Basic Technique, 4th Edition, Wiley-Liss 2000. In certain embodiments, the cells are cultured by monolayer culture.

Any medium capable of supporting adult stem cells in tissue culture may be used. Media formulations that will support such growth include, but are not limited to, Dulbecco's Modified Eagle's Medium (DMEM), alpha modified Minimal Essential Medium (.alpha.MEM), and Roswell Park Memorial Institute Media 1640 (RPMI Media 1640) and the like. Typically, 0 to 20% Fetal Bovine Serum (FBS) or 1-20% horse serum will be added to the above media in order to support the growth of adult stem cells. However, a defined medium could be used if the necessary growth factors, cytokines, and hormones in FBS are identified and provided at appropriate concentrations in the growth medium. Media useful in the methods of the invention may contain one or more compounds of interest, including, but not limited to antibiotics, mitogenic and differentiative compounds for adult stem cells. The cells will be grown at temperatures between 31° C. to 37° C. in a humidified incubator. The carbon dioxide content will be maintained between 2% to 10% and the oxygen content between 1% and 22%. Cells may remain in this environment for periods of up to 4 weeks.

Antibiotics which can be supplemented into the medium include, but are not limited to penicillin and streptomycin. The concentration of penicillin in the chemically defined culture medium is about 10 to about 200 units per ml. The concentration of streptomycin in the chemically defined culture medium is about 10 to about 200 μg/ml.

The Cells of the Invention have a Strong Tropism for their Tissue of Origin.

It is well known that hematopoietic stem cells have strong tropism for the bone marrow when administered into the systemic circulation. This characteristic has been extensively exploited to facilitate bone marrow reconstitution by the administration of the reconstituting cells trough the systemic circulation. To assess whether a similar trait was also expressed by the cells of the invention, 1×10⁶ Oct4 tripotent cells isolated from a rat heart which had undergone >50 passages and had been maintained in culture for more than 3 years and genetically tagged with a GFP expressing vector, where administered to each of 6 syngeneic rats. These rats had undergone the production of an acute myocardial infarction by ligation of the anterior descending coronary artery a few hours prior to the cell administration. A similar number of infarcted animals were injected with syngeneic GFP positive fibroblast An equal number of non-infarcted animals served as control. The animals were sacrificed two weeks later.

Up to 50% of the Oct4 positive cells were located in the damaged myocardium. These cells had been incorporated into the regenerating tissue and most differentiated into cardiac myocytes. Less than 10% of the GFP positive cells were found in other tissues, mainly the lung. Very few cells were found in the myocardium of the non-infarcted animals and even less in the animals transplanted with fibroblasts independently of whether they have been infarcted or not.

To further analyze this cardiac tropism, GFP positive cells of the invention maintained in culture for more than 5 years in any one of media I-IV as described above, were administered via the intra-coronary route to 20 allogeneic pigs after the production of an acute myocardial infarction by balloon occlusion of the anterior descending coronary artery. At 24, 72 and 168 hours after administration all the injected cells were located within the damaged myocardium. During the administration or at different times after, GFP negative cells could be detected in the systemic circulation or in any animal tissue, including lungs and liver, except in the myocardium. All the cells injected could be accounted for in the myocardium, particularly in the damaged area. In contrast, <5% of cells from the bone marrow injected by a similar procedure home to the myocardium (FIG. 22).

This characteristic of the cells of the invention can be exploited for the reconstitution of the stem cell population of different organs and/or tissues by administering cells directly into the tissue/organ or through the systemic circulation taking advantage of the tropism of the cells for their tissue of origin to increase their local concentration even through peripheral administration.

Therapeutic Use

The cells of the invention are suitable for cellular therapies, including the induction of tissue repair/regeneration in vivo. Therefore, the invention provides a method of treating a patient, wherein the method comprises administering cells of the invention to the patient in an appropriate amount.

Within one aspect, the isolated adult stem cells of the invention or progeny thereof can be used in medicine. In order to be used in medicine, the cells are generally formulated into a composition with a pharmaceutically acceptable carrier. For use in treatment, the cells are generally present in the form of single cells, rather than as clusters or collections of cells.

The pharmaceutically acceptable carrier may comprise a cell culture medium which supports the cells' viability. The medium will generally be serum-free in order to avoid provoking an immune response in the recipient. The carrier will generally be buffered and/or pyrogen-free.

Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. In many embodiments, the solution is stable under the conditions of manufacture and storage and preserved against the contaminating action of microorganisms such as bacteria and fungi through the use of, for example, parabens, chlorobutanol, phenol, ascorbic acid, thimerosal. This list is provided by way of illustration only, and is not intended to be limiting. Solutions that are adult stem cell compositions of the invention can be prepared by incorporating adult stem cells as described herein in a pharmaceutically acceptable carrier or diluent and, as required, other ingredients enumerated above, which has been sterilized by filtration.

Some examples of materials and solutions which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations. This list is provided by way of illustration only, and is not intended to be limiting.

Generally the cells of the invention or progeny thereof are introduced into the body of the patient by injection or implantation. Generally the cells will be directly injected into the tissue in which they are intended to act. A syringe containing cells of the invention and a pharmaceutically acceptable carrier is included within the scope of the invention. A catheter attached to a syringe containing cells of the invention and a pharmaceutically acceptable carrier is included within the scope of the invention.

As discussed above, the adult stem cells of the invention can be used in the regeneration of tissue. In order to achieve this function, cells may be injected or implanted directly into the damaged tissue, where they multiply and eventually differentiate into the required cell type, in accordance with their location in the body. Tissues that are susceptible to treatment include all damaged tissues, particularly including those which may have been damaged by disease, injury, trauma, an autoimmune reaction, or by a viral or bacterial infection.

In one embodiment the cells of the invention or progeny thereof, either in solution, in microspheres or in microparticles of a variety of compositions, will be administered into the artery irrigating the tissue or the part of the damaged organ in need of regeneration. Generally such administration will be performed using a catheter. The catheter may be one of the large variety of balloon catheters used for angioplasty and/or cell delivery or a catheter designed for the specific purpose of delivering the cells to a particular local of the body. Although the cells exhibit a strong tropism for certain tissues (e.g. myocardium) for certain indications it may be desirable to note that most of the cells administered to the patient do not go through the capillary network and into the systemic circulation. For certain uses, the cells may be encapsulated into microspheres made of a number of different biodegradable compounds, and with a diameter of about 15 μm. This method may allow intravascularly administered cells to remain at the site of damage, and not to go through the capillary network and into the systemic circulation in the first passage. The retention at the arterial side of the capillary network may also facilitate their translocation into the extravascular space.

In another embodiment, the cells may be retrograde injected into the vascular tree, either through a vein to deliver them to the whole body or locally into the particular vein that drains into the tissue or body part to which the cells are directed. For this embodiment many of the preparations described above may be used.

An alternative embodiment for the treatment of the myocardium is a transcatheter injection transendocardically, with or without electric mapping with a system such as the Noga system or any similar injection system.

In another embodiment, the cells of the invention or progeny thereof may be implanted into the damaged tissue adhered to a biocompatible implant. Within this embodiment, the cells may be adhered to the biocompatible implant in vitro, prior to implantation into the patient. As will be clear to a person skilled in the art, any one of a number of adherents may be used to adhere the cells to the implant, prior to implantation. By way of example only, such adherents may include fibrin, one or more members of the integrin family, one or more members of the cadherin family, one or more members of the selectin family, one or more cell adhesion molecules (CAMs), one or more of the immunoglobulin family and one or more artificial adherents. This list is provided by way of illustration only, and is not intended to be limiting. It will be clear to a person skilled in the art, that any combination of one or more adherents may be used.

In another embodiment, the cells of the invention or progeny thereof may be embedded in a matrix, prior to implantation of the matrix into the patient. Generally, the matrix will be implanted into the damaged tissue of the patient. Examples of matrices include collagen based matrices, fibrin based matrices, laminin based matrices, fibronectin based matrices and artificial matrices. This list is provided by way of illustration only, and is not intended to be limiting.

In a further embodiment, the cells of the invention or progeny thereof may be implanted or injected into the patient together with a matrix forming component. This may allow the cells to form a matrix following injection or implantation, ensuring that the cells remain at the appropriate location within the patient. Examples of matrix forming components include fibrin glue liquid alkyl, cyanoacrylate monomers, plasticizers, polysaccharides such as dextran, ethylene oxide-containing oligomers, block co-polymers such as poloxamer and Pluronics, non-ionic surfactants such as Tween and Triton‘8’, and artificial matrix forming components. This list is provided by way of illustration only, and is not intended to be limiting. It will be clear to a person skilled in the art, that any combination of one or more matrix forming components may be used.

In a further embodiment, the cells of the invention or progeny thereof may be contained within a microsphere. Within this embodiment, the cells may be encapsulated within the centre of the microsphere. Also within this embodiment, the cells may be embedded into the matrix material of the microsphere. The matrix material may include any suitable biodegradable polymer, including but not limited to alginates, Poly ethylene glycol (PLGA), and polyurethanes. This list is provided by way of example only, and is not intended to be limiting.

In a further embodiment, the cells of the invention or progeny thereof may be adhered to a medical device intended for implantation. Examples of such medical devices include stents, pins, stitches, splits, pacemakers, prosthetic joints, artificial skin, and rods. This list is provided by way of illustration only, and is not intended to be limiting. It will be clear to a person skilled in the art, that the cells may be adhered to the medical device by a variety of methods. For example, the cells may be adhered to the medical device using fibrin, one or more members of the integrin family, one or more members of the cadherin family, one or more members of the selectin family, one or more cell adhesion molecules (CAMs), one or more of the immunoglobulin family and one or more artificial adherents. This list is provided by way of illustration only, and is not intended to be limiting. It will be clear to a person skilled in the art, that any combination of one or more adherents may be used.

In another embodiment the cells of the invention can be administered into the peripheral circulation and through their tropism for the tissue of origin it can be expected that the cells will home to the organ/tissue to be treated.

As described above, the cells of the present invention can be induced to differentiate into any cell type. Such differentiation can be induced either in vitro or in vivo. The therapeutic application may thus require injection or implantation of non-differentiated adult stem cells, which will be induced to differentiate within the tissue. Alternatively, the adult stem cells of the invention can be induced to differentiate into the required cell type in vitro, with the subsequent injection of a pharmaceutically acceptable composition comprising these cells into the damaged tissue.

Due to the high level of growth factors, cytokines and chemokines produced by the cells of the invention, the cells can be used not only for their own differentiation properties but also for their paracrine activities. This allows the cells of the invention to activate the resident stem cells of the tissue to be treated, and may result in a reduction of the inflammatory reaction of the tissue and/or a decrease in the amount of cell death and/or a stimulation of the rate of cell survival of the treated tissue. Such paracrine activities may be induced by the introduction of the cells into the damaged tissue, as already described for several embodiments or by the local application of the cells into hollow organs or onto the external surface of the body for the treatment of a variety of different conditions, including but not limited to chronic ulcers and wound healing in general.

For all the embodiments so far described the cells administered may be either autologous, immunologically matched or heterologous.

The heterologous approach has the advantage that the differentiating cells will be rapidly eliminated by the immune system, thus reducing the risk development of either teratomas or neoplasias derived from the transplanted cells or their descendants. Within this embodiment, the use of heterologous cells may result in the autologous regeneration of the treated tissue, through the paracrine induced stimulation, multiplication and differentiation of the resident stem cells of the recipient.

Autologous or heterologous cells of the invention may be administered systemically either through the arterial or venous route for the treatment of generalized conditions such autoimmune diseases. Within this embodiment, the number of cells administered may be in the range of 1×10⁸ cells per kg of body weight.

The cells of the invention are also suitable for inducing tissue regeneration ex vivo, for instance in tissue prior to transplantation. Thus the invention provides an ex vivo method for modifying tissue, comprising adding a cell of the invention to the tissue. This method allows the adult stem cells of the invention to be differentiated and to propagate and repair the damaged tissue, prior to transplantation.

For use in medicine, the cells will be delivered to the patient in a therapeutically effective amount. The number of cells to be delivered in vivo or ex vivo is based on a number of parameters, including: the body weight of the patient, the severity of tissue damage, and the number of cells surviving within the subject. A typical number of cells may be around 10⁶ to 10⁹ cells, more particularly 10⁷ to 10⁸ cells per kg body weight. It may be necessary to repeat injection or implantation of the cells over several months to achieve the necessary cumulative total mass and/or to replace cells which are dying. Generally, the total number of cells delivered to the patient in a single treatment regiment will be greater than about 1×10⁸. However, the total number of cells delivered may be higher than 1×10¹⁰.

In one embodiment, the isolated adult stem cells may be used in the regeneration of cardiac tissue, including in the regeneration of myocardium. In this embodiment, the cells of the invention may be injected or implanted directly into the damaged cardiac tissue trans-endocardically; using a needle catheter which injects the cells into the myocardium, intra-arterially; using a balloon catheter into the artery irrigating the damaged tissue area, or retrograde; by injecting the cells into the coronary vein draining the damaged area.

In one embodiment, the adult stem cells that are used in any of the methods described above may be autologous with respect to the patient being treated. Within this embodiment, the adult stem cells are isolated from the body of the patient, which may be according to one of the methods described above. These cells are cultured and formulated into a pharmaceutically acceptable composition as described above, before being introduced into the patient from whom they were originally removed, which may be by injection or implantation.

Within a further aspect of the invention, the isolated adult stem cell is heterologous and, therefore, allogeneic with respect to the patient being treated. Within this embodiment, the cells are isolated from a subject, which may be according to one of the methods described above. Generally, the patient will have to be matched with the subject from whom the adult stem cells originated, for example, by MHC haplotype or in some other way in order to avoid rejection. These cells are then cultured and formulated into a pharmaceutically acceptable composition as described above, before being introduced into a patient who is different from the subject from whom they were originally removed, by injection or implantation. As already indicated, unmatched cells may be used to exploit the advantage that the unmatched administered cells and their descendants will be completely eliminated in a matter of weeks, thus eliminating the risk of teratoma formation or the appearance of late neoplasias originating from the transplanted cells or their descendants.

Further within the invention, the isolated adult stem cell is used in the manufacture of a medicament for use in tissue regeneration, including but not limited to regeneration of the liver, certain areas of the brain, such as those associated with Parkinson's disease, and the pancreas. Such regeneration may allow the treatment of Parkinson's disease, type II diabetes, chronic skin ulcers, and autoimmune disorders. This list is provided by way of illustration, and is not intended to be limiting. In certain embodiments, the medicament is for use in the regeneration of cardiac tissue, and may be used for the treatment of any pathology that would benefit from the availability of more and/or better functioning contractile cells (myocytes) or microvasculature, including but not limited to acute myocardial infarction, chronic ischemic cardiomyopathy, cardiomyopathy, and chronic heart failure. This list is provided by way of illustration only, and is not intended to be limiting.

Drug Testing

Many of the long short and long term side effects of drugs administered systemically can be attributed to their deleterious effect on the cell homeostasis of either the target tissue or on other tissues. The tissue stem cells are an essential component of cell homeostasis. The cells of the invention provide a convenient in vitro system where the effect of different drugs as well as antidotes and molecules able to stimulate the multiplication and/or differentiation of the stem cells of a particular tissue can be tested.

### EXAMPLES

### Murine Adult Stem Cells

### Example 1

### Isolation and Culture of Murine Cardiac Stem Cells

The following mouse strains were used to isolate stem cells: Oct4-EGFP (B6; CBA-Tg(Pou5f1-EGFP)2Mnn/J) transgenic mice (29), Rosa26 mice (B6.129S7-Gt(ROSA)26Sor/J) and the inbred C57BL/6 strain (The Jackson Laboratories).

The hearts of adult male mice (4-8 weeks old) were retrogradely perfused through the aorta with 50 ml of 1 mg/ml collagenase type II (300-330 U/mg; Worthington Biochemical Corporation) in Basic Medium: 0.7 g/l Hepes, 0.3 g/1 L-glutamine, 1.25 g/l taurine, 20 U/l insulin, 50 μg/ml gentamycin, 0.1 mg/ml streptomycin and 100 U/ml penicillin (Sigma); 250 ng/ml amphotericin B, 205 ng/ml sodium deoxycholate (Fungizone, Invitrogen) in Eagle's Minimum Essential Medium, Joklik modification (MEM, Sigma), pH 7.3. The perfusion was performed at 37° C. using a distilling column (Ace Glass) coupled to a water bath circulator and a peristaltic pump (Cole-Parmer) that assured a 2.5 ml/min flow rate. After the perfusion with collagenase, the heart was recovered in 0.5% bovine serum albumin (BSA, Sigma) in Basic Medium, at 4° C., washed once, transferred into a sterile beaker under the laminar flow hood and minced into little pieces using scissors. The tissue was mechanically dispersed with a Pasteur plastic pipette. The supernatant was filtered with 40 μm nylon filters (Falcon) and centrifuged 5 min at 4° C., 300 g. The resulting pellet was resuspended in PBS-BSA at 4° C.: 0.5% BSA (Sigma); 2 mM ethylenediaminetetraacetic acid (EDTA, Invitrogen); 50 μg/ml gentamycin, 0.1 mg/ml streptomycin, 100 U/ml penicillin (Sigma); 250 ng/ml amphotericin B, 205 ng/ml sodium deoxycholate (Fungizone, Invitrogen) in phosphate buffer (PBS, Invitrogen), pH 7.2. The cell suspension was incubated for 20 min at 4° C. with a 1:5 dilution of an anti-ckit rat monoclonal antibody conjugated with R-phycoerythrin (Miltenyi Biotec).

The cell suspension was washed once with PBS-BSA at 4° C. and resuspended in the same solution, adding 1 μg/ml propidium iodide (Sigma) in order to discard non-viable cells during the cell sorting. Viable c-kit^{pos} cells were separated by fluorescence-activated cell sorting (FACS) using a high speed MoFlo cell sorter (Cytomation).

For clonal studies, cells were directly sorted in 96-well gelatine-coated plates (Becton Dickinson), with 1 cell/well. Daily screening of the wells allowed verification of the presence of a single cell in each well. Upon reaching 40% confluency, the cells were serially seeded in gelatin-coated 6-well plates and 100 mm dishes. Subsequently, the cells were passaged with 1:5 dilutions. The clones were expanded in vitro and the most rapidly growing ones were selected for further analysis.

Oct4 CSCs isolated from rats were obtained by the same technique.

Cardiac c-kit^{pos} cells were cultured in culture dishes coated with 0.2% gelatine in culture medium at 37° C. and 5% CO₂ in a water-jacketed incubator. Four different media were assayed for their ability to sustain the undifferentiated state of cardiac c-kit^{pos} cells in long-term cultures. Overall, fibroblast growth factor-2 (FGF-2) and epidermal growth factor (EGF), together with leukaemia inhibitory factor (LIF), proved to promote symmetrical self-renewing divisions and to allow the long-term propagation of undifferentiated cardiac c-kit^{pos} cells as adherent cultures on gelatin-coated dishes.

Standard ESC culture conditions were used for growth of mouse ES-D3 mouse embryonic stem cells (ESCs, ATCC).

### Example 2

### C-kit^{pos} Cells Isolated from the Adult Murine Heart Express Multipotency Genes

Following isolation of the cardiac stem cells, they were tested for expression of various markers involved in self-renewal, pluripotency and balance between the undifferentiated and committed state of ESCs.

To this end, total RNA was extracted using Trizol (Invitrogen) followed by DNAse treatment with TURBO DNA-free™ (Ambion) to eliminate contaminating genomic DNA. The obtained RNA was further purified using the RNeasy mini kit (Qiagen). Conventional reverse transcription, using the MultiScribe reverse transcriptase (Applied Biosystems), was performed following the manufacturer's recommendations.

Semiquantitative real-time PCR was performed using SYBR® GREEN (Applied Biosystems) on an ABI PRISM® 7900HT Sequence Detection System (Applied Biosystems). Primers were designed using the Primer Express software (Applied Biosystems) and when possible were selected to span introns to further avoid amplification of contaminating genomic DNA. A primer concentration of 300 nM was found to be optimal in all cases.

The PCR protocol consisted of 1 cycle at 95° C. (10 min) followed by 40 cycles of 95° C. (15 s), 55-62° C. (1 min). A dissociation curve analysis was included after each experiment to confirm the presence of a single product and the absence of primer dimers. Also, a standard curve using multiple concentrations of cDNAs was designed for each sample and pair of primers to assure that the amplification efficiency was similar among the different samples. GAPDH expression was used as a standard. The average threshold cycle number (Ct) for each tested mRNA was used to quantify the relative expression of each gene, following the equation 2—(Ctgene−CtGAPDH).

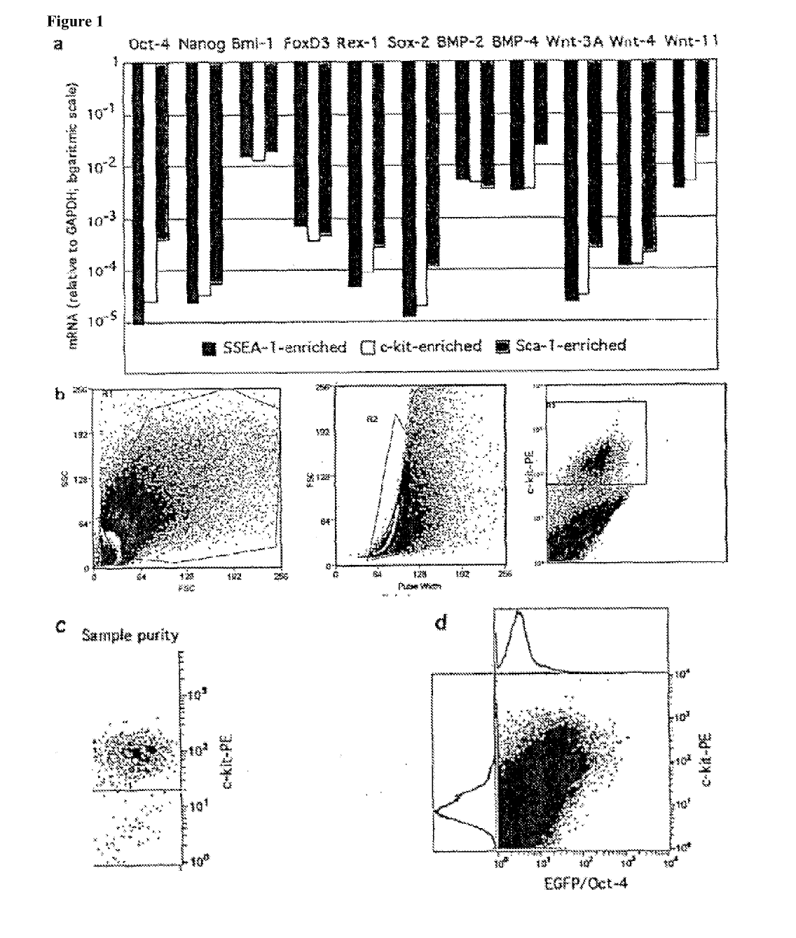

Surprisingly, transcripts for the homeodomain protein Oct4, Nanog, Klf-4, the acidic zinc finger protein Rex-1, the SRY-related HMG box transcription factor Sox-2, the winged-helix transcription factor FoxD3, the Polycomb group protein Bmi-1, the bone morphogenetic proteins BMP-2 and BMP-4, the Wnt family members Wnt-3A, Wnt-4 and Wnt-11 could be amplified in samples enriched for the expression of the stage-specific embryonic antigen-1 (SSEA-1), the receptor for the stem cell factor (c-kit) or the stem cell antigen-1 (Sea-1) (FIG. 1), indicating that these adult cells possess characteristics of embryonic stem cells.

The expression of the major multipotency genes at the mRNA level was confirmed at the protein level, as shown in FIG. 23.

### Example 3

### Direct Enrichment and Isolation of Oct4 Expressing Cells from the Adult Murine Myocardium

Hearts of 6 week old B16J mice were retrogradely perfused through the coronary arteries in a manner and with solutions identical to those described for Example 1. The c-kit positive cells present in the small cell population were also isolated as described above. The cell suspension was washed once with PBS-BSA at 4° C. and resuspended in the same solution, adding 1 μg/ml propidium iodide (Sigma) in order to discard non-viable cells during the cell sorting. Viable cells double positive for c-kit and SSEA1 were separated by fluorescence-activated cell sorting (FACS) using a high speed MoFlo cell sorter (Cytomation). On overage ˜2×10³ double positive cells, c-kit^{pos} SSEA1^{pos}, were isolated per mouse heart, which routinely resulted in >1×10⁴ double positive cells from each pool of 6 hearts.

The double positive cells were either pooled or plated at a concentration of 0.5 cell per well in gelating coated Terasaki plates for the development of clones. An aliquot of the pooled cells was placed on slides by cytospin and analyzed by immunocytochemistry for the expression of the multipotency genes. On overage 65-85% of the cells expressed Oct and Nanog at the protein level.

Aliquots from the clones originated from either the direct plating from the cell sorter or from the pooled cell cultures were analyzed for the expression of the multipotency genes, expanded and either used for further analyses or stored frozen.

### Example 4

### Direct Isolation of Oct4 Cells from Adult Murine Tissue by Selection with an Oct4 and Nanog Reporter Gene Constructs

We have prepared reporter vectors with either GFP or YFP driven by the promoter sequences from the human Oct gene (sequence from nucleotide −3916 to nucleotide −1, just before the initiation of transcription site) or the human Nanog gene promoter (sequence from nucleotide −416 to nucleotide 0). These reporters have been engineered into lentiviruses and viral suspension of high titre prepared according to standard techniques and procedures known to all practitioners of techniques of molecular biology.

c-kit^{pos} cells isolated from either heart, bone marrow or brain from murine tissues by the procedures outlined in the previous examples are plated at low density in growth medium and when attached (between 48 and 72 hours after plating), the cells are transfected with one of the letivirus preparations at a PFU of 10 to 1 using standard protocols for lentiviral infection of mammalian cells. Seventy two hours after the transfection the Oct4 and/or Nanog positive cells can be identified under the fluorescent microscope for either their green or yellow fluorescence. At the desired time the positive cells can be sorted based on their fluorescence and, if desired, cells with high level expression of the marker gene (high fluorescence) specifically sorted. From cell preparations obtained from 6 B16J mice at 6 weeks we isolated 6×10³ Oct4 positive and 5×10⁶ Nanog positive cells from the heart; 3×10³ Oct4 and 2×10³ Nanog positive cells from the bone marrow, and 4×10³ Oct4 and 1×10⁴ Nanog positive cells from the brain. These cells cloned at a very high frequency (>35%) and the clones had similar characteristics as those isolated by other procedures, as described in Example 1. These were no detectable differences between the cells isolated with the Oct4 and the Nonog reporter gene.

### Example 5

### Drug Selection of Cells from Different Murine Tissue Expressing Multipotency Genes

The lentiviral constructs described in Example 4 have been engineered to express the puromaycin resistance gene downstream from the fluorescence protein sequence. These two sequences are linked by and IRES (internal ribosome entry site) forming a polycystronic gene which produces the fluorescent and the drug resistance protein. In this manner, the cells of choice can be selected by growing the culture in a concentration of puromycin that will be lethal or all the cells not expressing the resistance gene (in the case of the murine and human c-kit^{pos} cells there are no survivors in the cultures containing 1 μg/ml puromycin).

c-kit^{pos} cells from mouse hearts prepared as described above were plated at low density in growth medium and when attached were transfected with an Oct-GFP-Puro lentivirus at a PFU of 10 to 1. Three days later the 1 μg/ml puromycin was added to the medium. Two weeks later the plates contained multiple GFP positive clones. Some of these clones were expanded and the phenotype of the cells analyzed by immunohistochemistry and rtPCR. >85% of the clones analyzed had a phenotype indistinguishable from that of the clones isolated in the prior examples described.

### Example 6

### Selection of Murine Cells Expressing Multipotency Genes from Bone Marrow and Myocardium

c-kit positive cells isolated from the myocardium of 8 weeks old Oct4-EGFP (B6; CBA-Tg(Pou5f1-EGFP)₂Mnn/J) transgenic mice (29) were plated at a density of 1×10⁴ cells/cm² in growth medium (see Example 1). After 72 hours, when the majority of the cells had attached, the medium was replaced with fresh growth medium supplemented with 2% FCS instead of the standard 10%. The cells were incubated for the next 10 days without changing the medium. At this time nascent clones of rounded cells had appeared in all the wells. These cells formed small “bunches of grapes” in the next few days which were uniformly expressing GFP as detected by direct immunofluorescence and by histoimmunofluorescence (FIG. 21). A total of 10 clones were picked mechanically, the cells expanded and their phenotype analyzed by rtPCR and immunocytology. All the clones analyzed have similar phenotype to each other and to the clones isolated by the methods described in the examples 1-5.

The description of the methods successful for the isolation, cloning and expansion of the multigene-expressing c-kitpos cells has been limited to those obtained from heart, bone marrow and brain of mouse tissues. The same protocols, however, have proven successful for the isolation of similar cells from the rat, pig and human, as well as from brain, skeletal muscle, and liver.

### Example 7

### Selection of Murine Cells Expressing Multipotency Genes from Brain, Skeletal Muscle and Liver

c-kit^{pos} Oct-4^{pos} cells from mouse brain, skeletal muscle and liver were isolated according to the protocols previously described. Specifically, for isolation of the cells from the liver, the organ was perfused retrogradely to remove most of the blood cells from the tissue. After the elutant from the tissue became clear, 3 gr of a lobe of the liver were finely minced and dounce homogenized with a loose pestle until a cell suspension was obtained. The parenchymal, epithelial and connective tissue cells were removed by differential centrifugation and the population of very small cells isolated. After further enrichment by filtration through a filter mesh with 10 μm diameter, the cells were processed as described in the isolation protocols section. From 3 gr. of minced donor liver in MEM medium, the CD45^{pos} and 34^{pos} were removed with the appropriate Mylteni beads which yielded 17×10⁶ cells. The c-kit^{pos} cells were isolated using an anti mouse c-kit antibody and a Mylteni column as described for Example 1. A total of 1.3×10⁶Lin^{neg} c-kit^{pos} were obtained from the whole sample. Analysis by immunohistochemistry of these cells after cytospinning them of slides showed that ˜1% of the cells were Oct4^{pos} Nanog^{pos}. An aliquot of these cells was plated for cloning. At three weeks the cloning efficiency was 11% of the plated cells. Five clones were selected for expansion and further characterization. All 5 clones showed expression of all major multipotency genes as well as TERT. One of these clones was tested in vitro for its regenerative capacity (see Example 23).

To isolate the cells from skeletal muscle, the thigh muscle of a donor mouse were injected with cardiotoxin according to standard protocols used routinely to induce skeletal muscle regeneration. Five days later, the muscles were dissected, and processed in a manner identical to the myocardial tissue samples after digestion with collagenase. The small cells were isolated by differential centrifugation sollowed by filtration, removal of the CD45^{pos} and 34^{pos} cell cohort with Mylteni beads. With the appropriate anti-c-kit antibody we obtained 0.9×10⁶ Lin^{neg} c-kit^{pos}. Of these, 4% were Oct4^{pos} Nanog^{pos} when examined by immunohistochemistry.

To isolate neural Oct4^{pos} Nanog^{pos} cells, the frontal lobes (including the olfactory bulbs) and the paraventricular zones of a single mouse were dissected and processed separately. After douncing the cells suspensions were processed exactly as described for the liver and skeletal muscle tissues. We obtained 0.6×10⁶ and 0.4×10⁶ Lin^{neg} c-kit^{pos} from the frontal lobe and paraventricular zones, respectively. From these 9 and 11% were Oct4^{pos} Nanog^{pos}. Several clones from each brain region were obtained which exhibited high level of expression of the multipotency genes as shown in FIG. 24. From a phenotypic point of view the cells obtained from the frontal lobe were not distinguishable from those originated from the paraventricular tissue.

### Example 8

### Oct4^{pos} Cells are Present Throughout the Adult Murine Myocardium

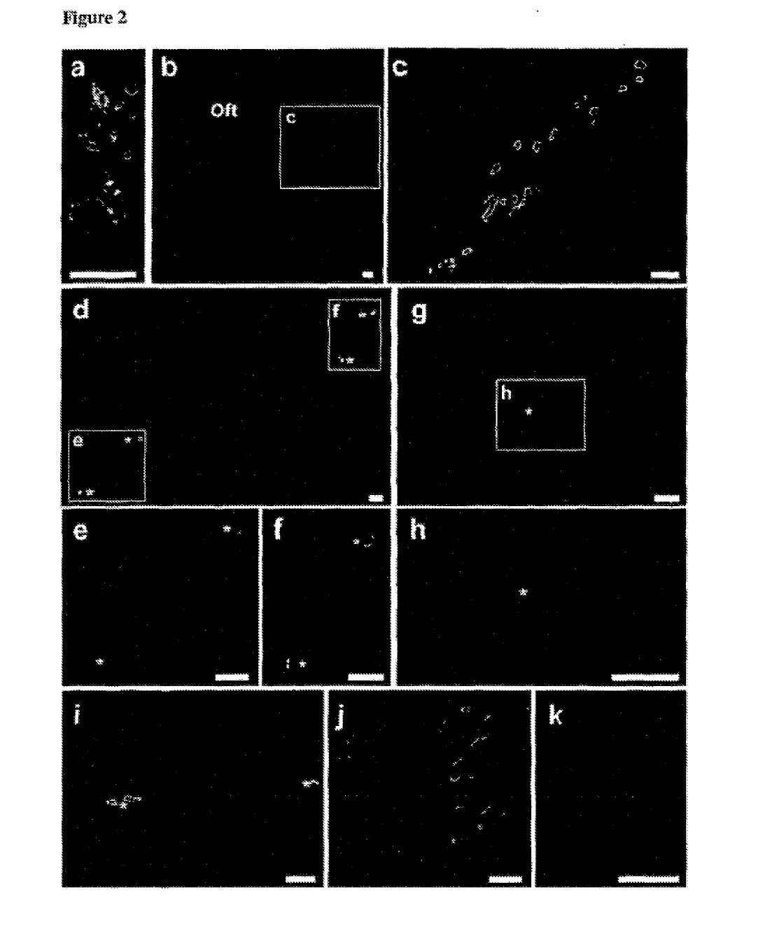

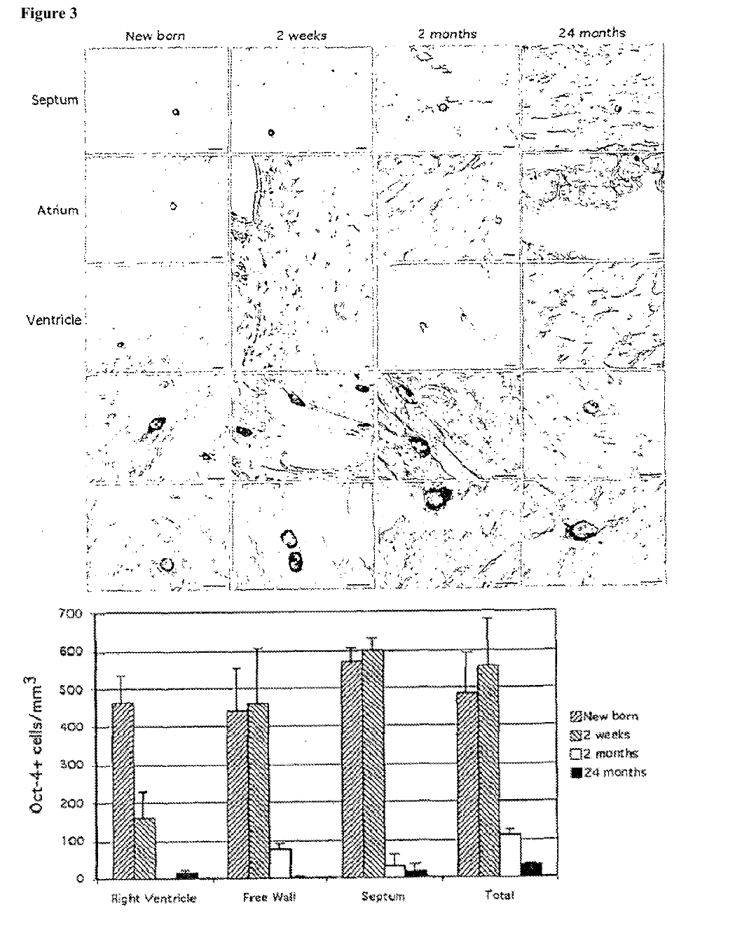

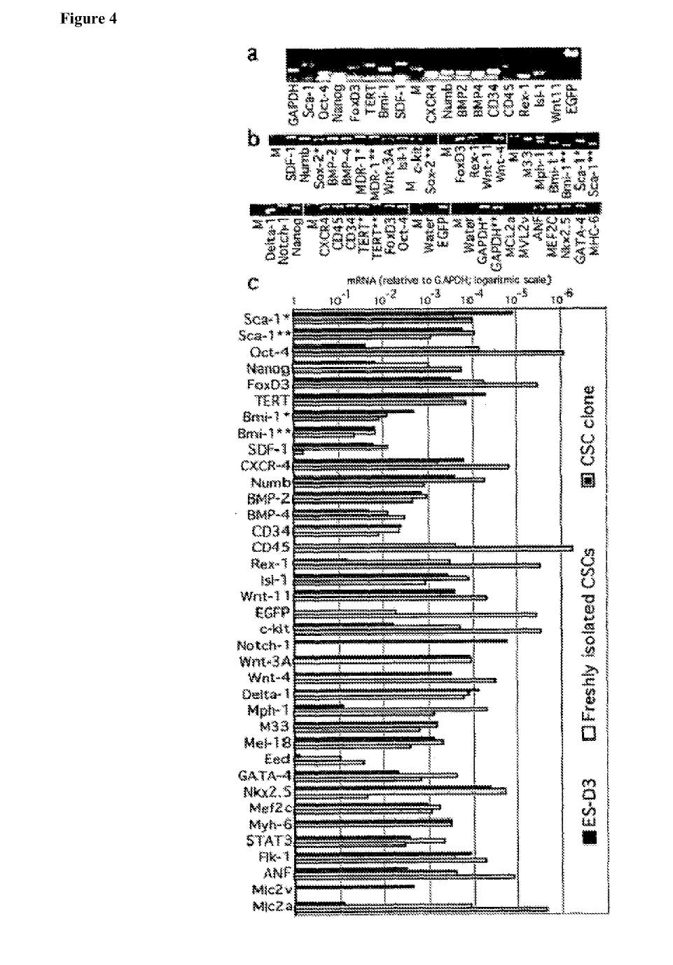

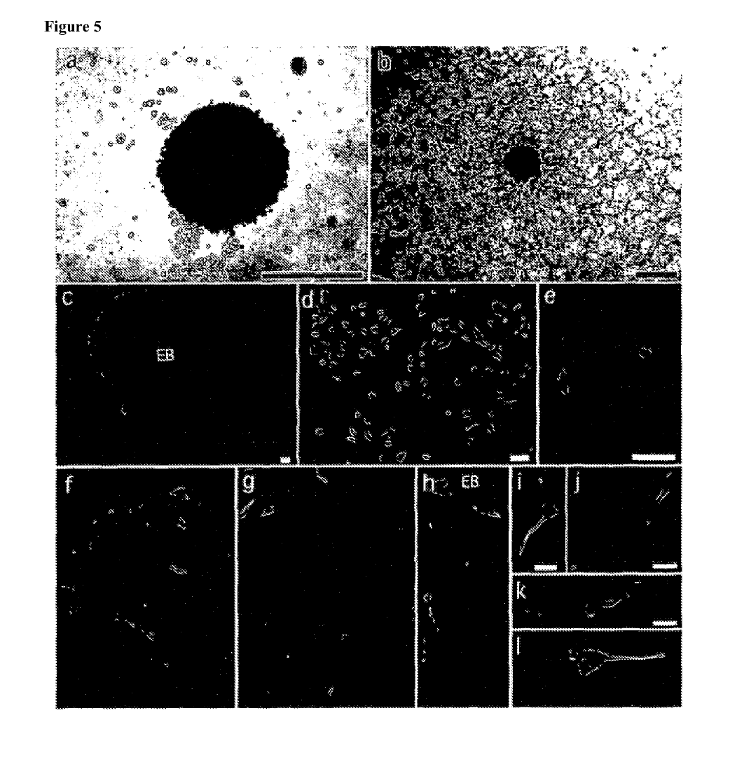

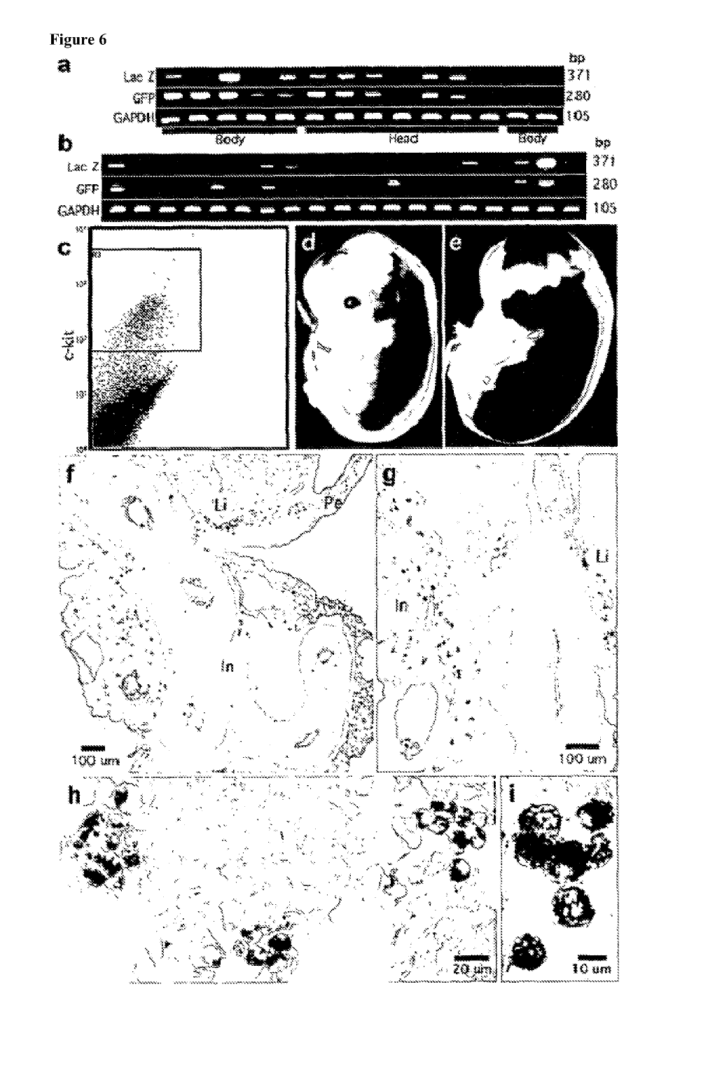

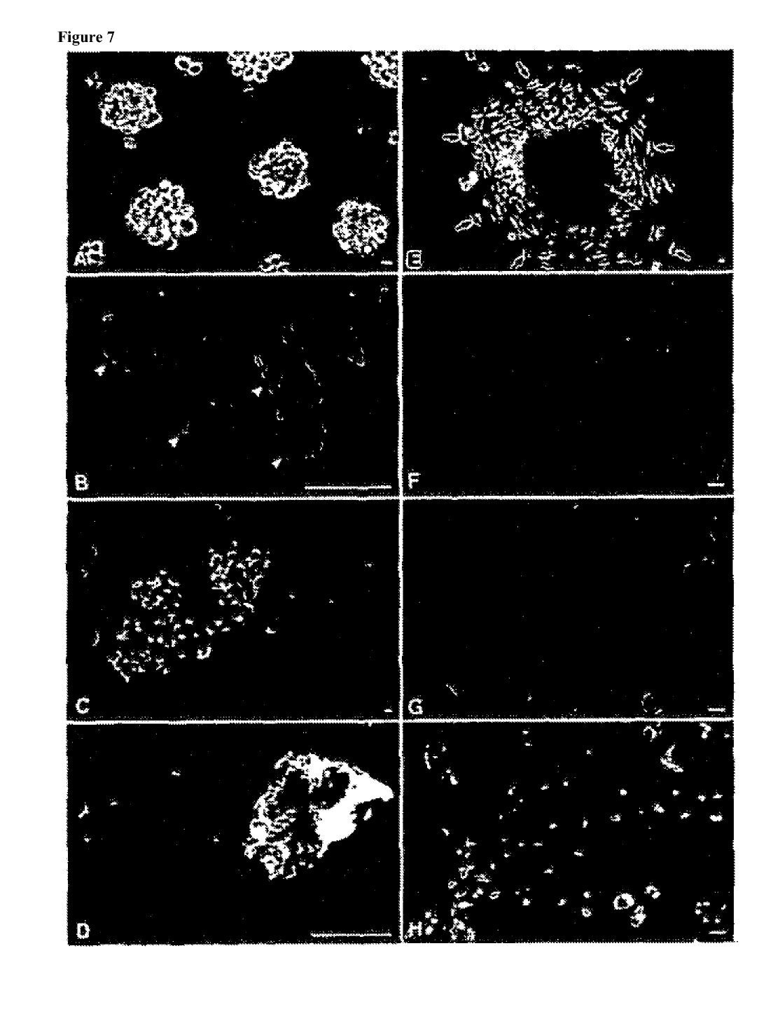

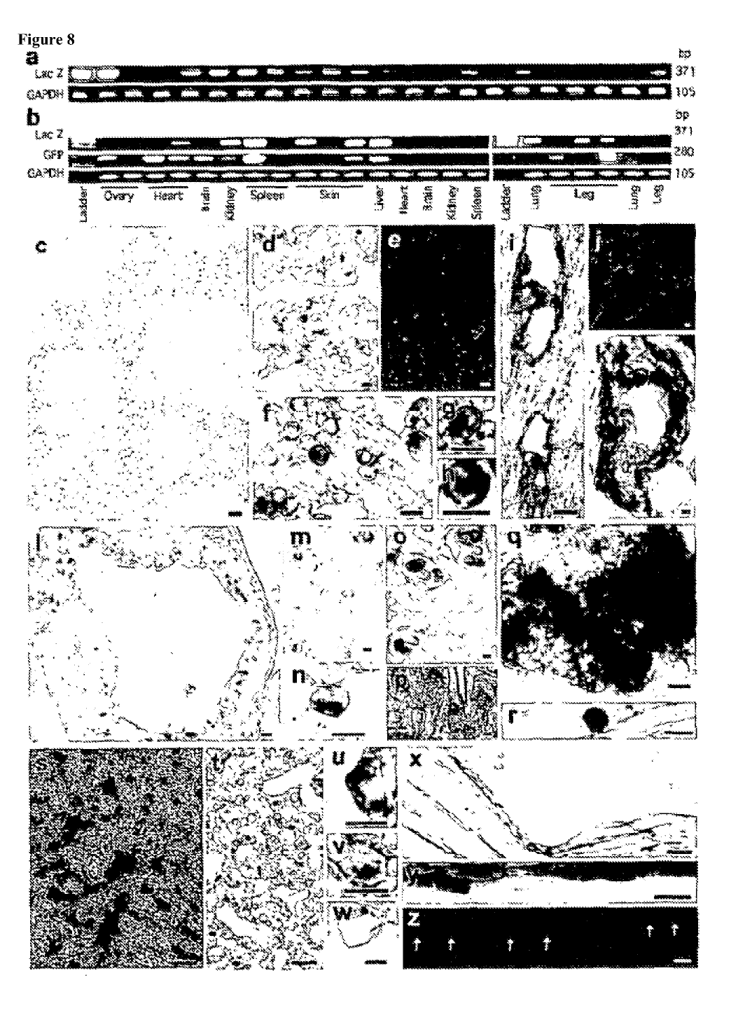

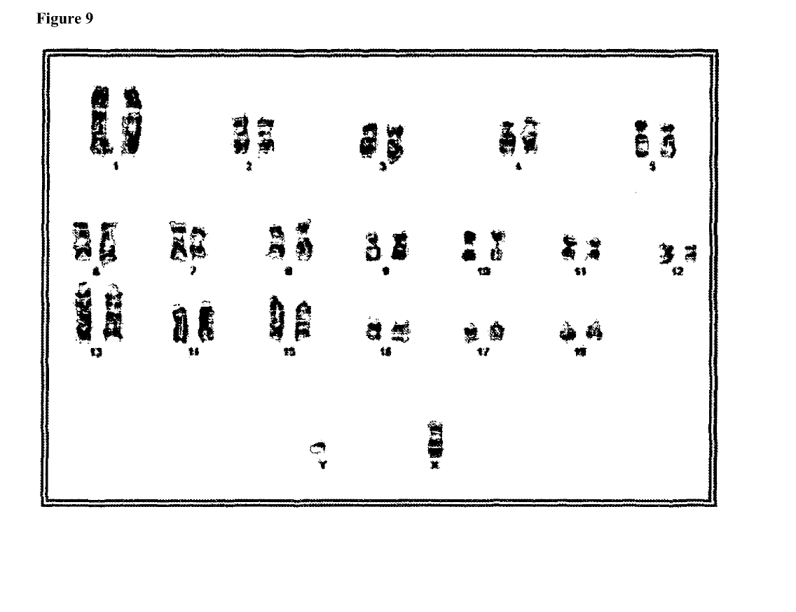

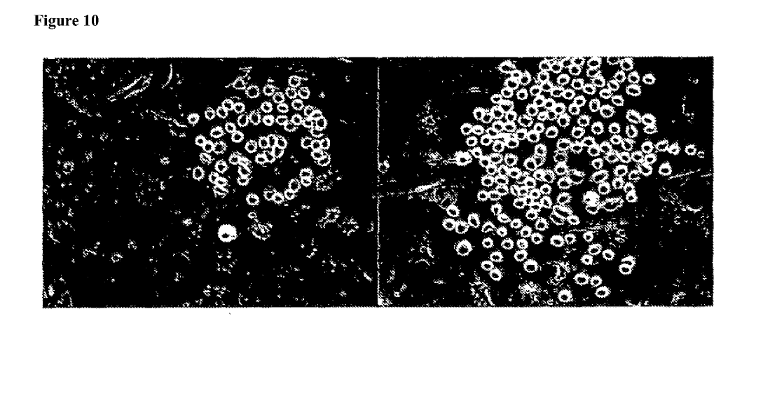

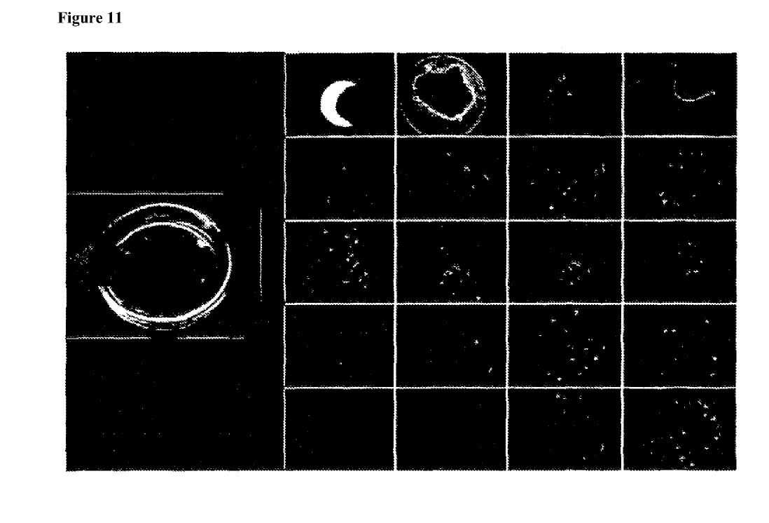

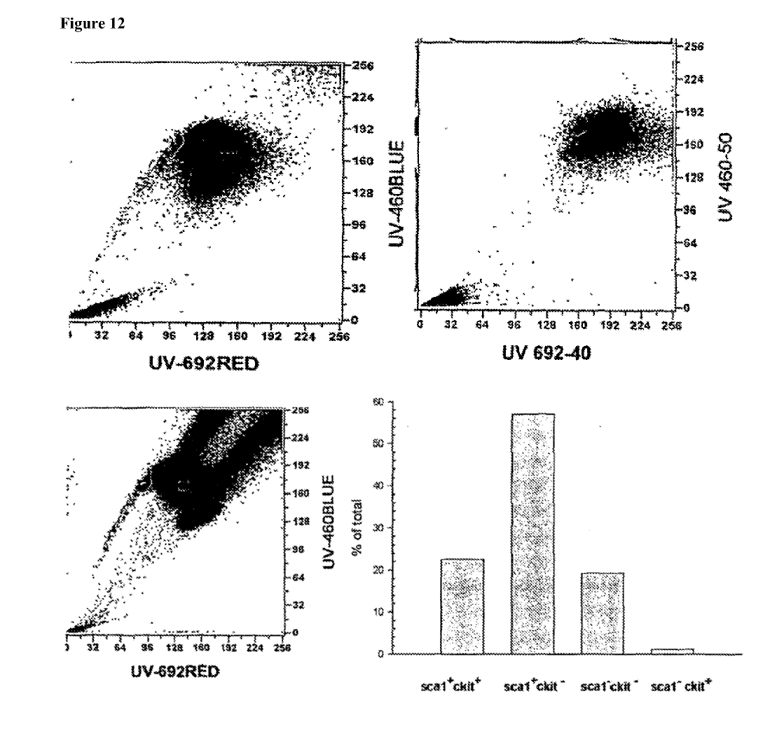

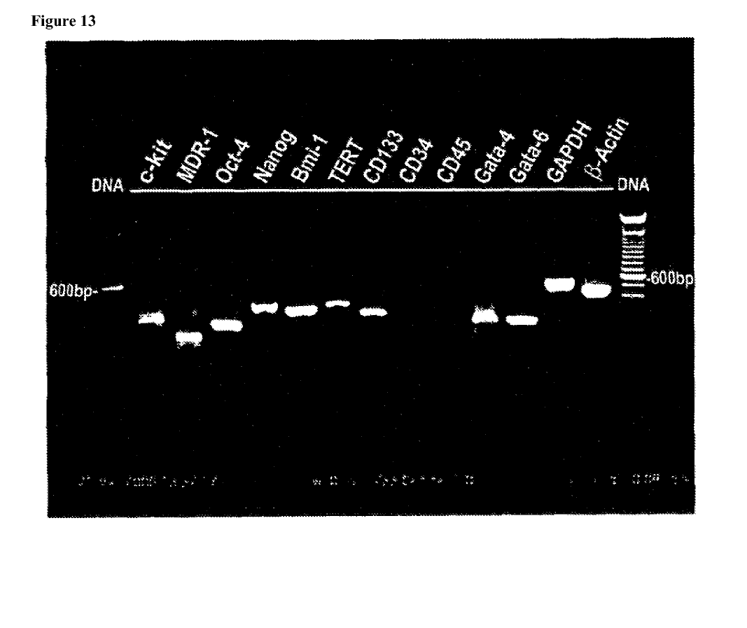

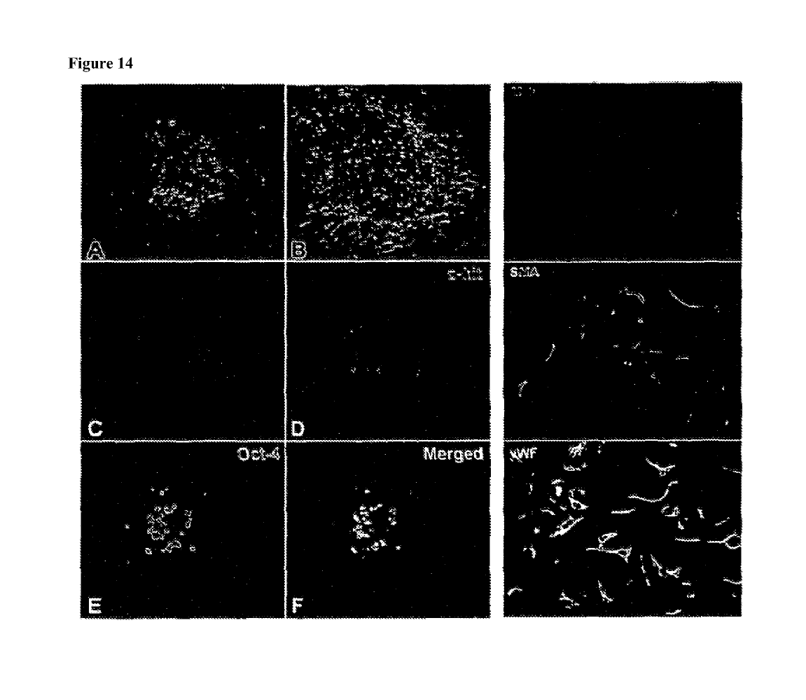

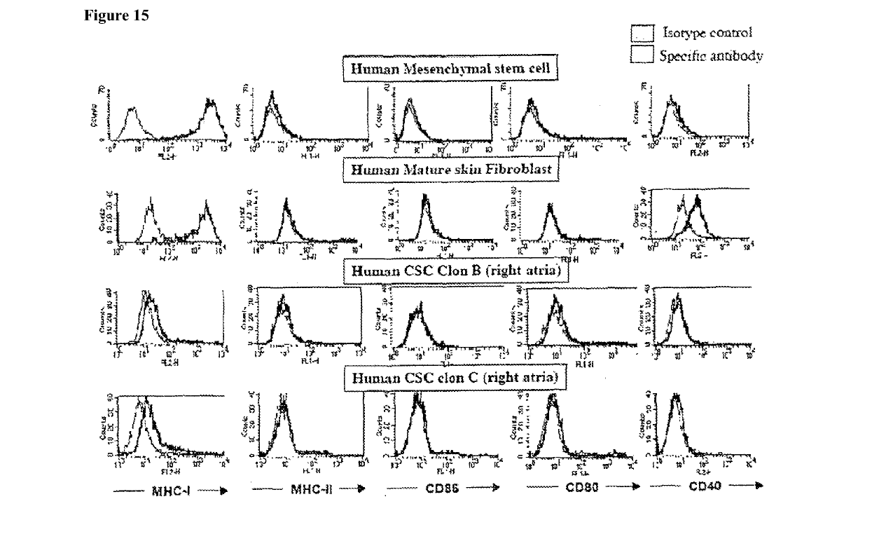

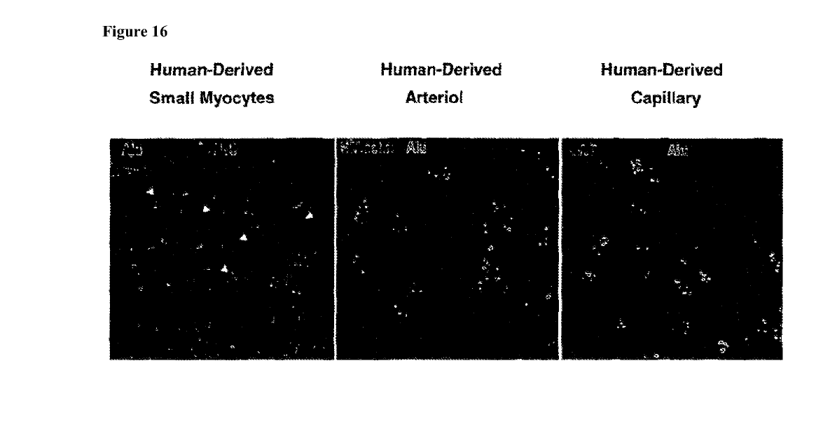

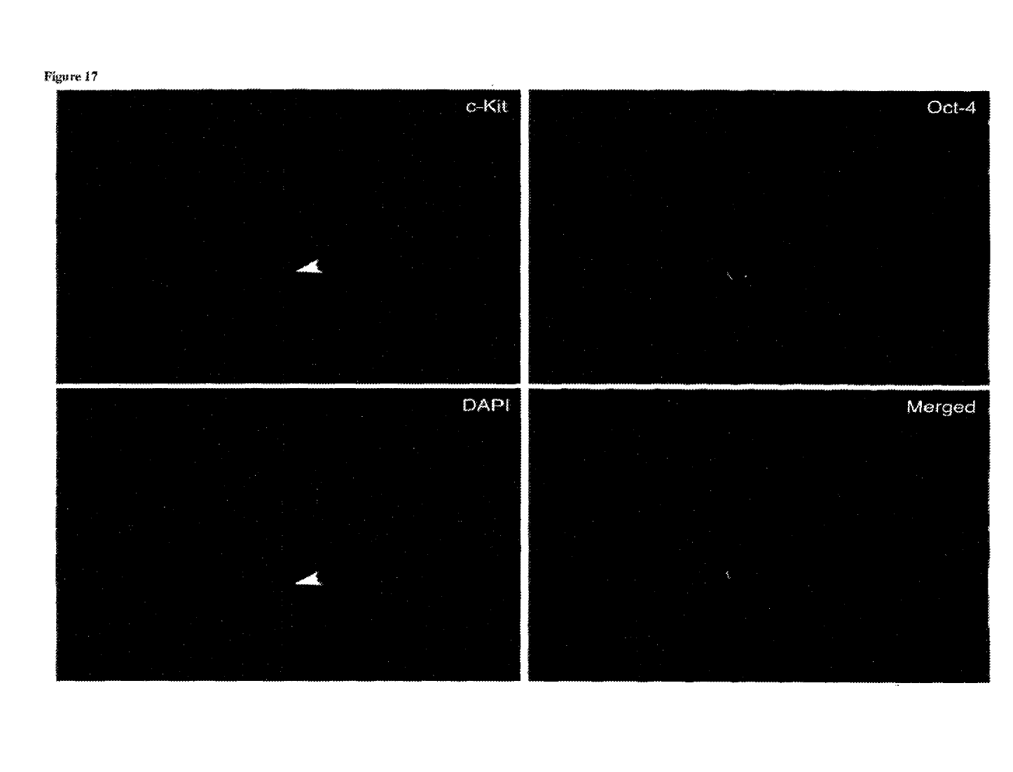

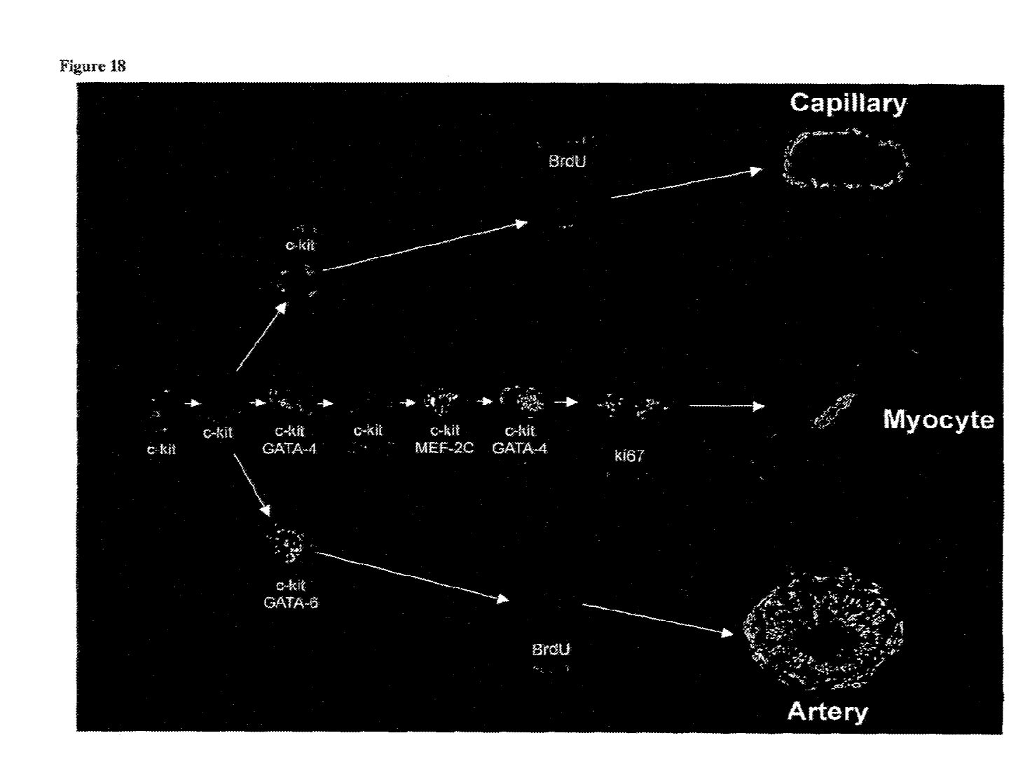

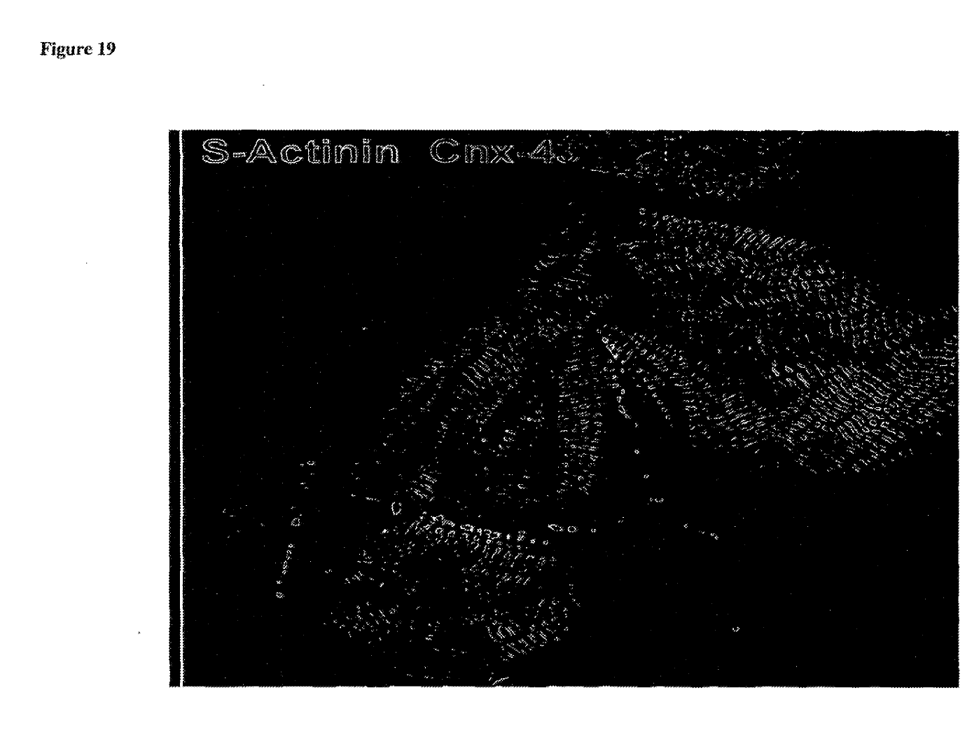

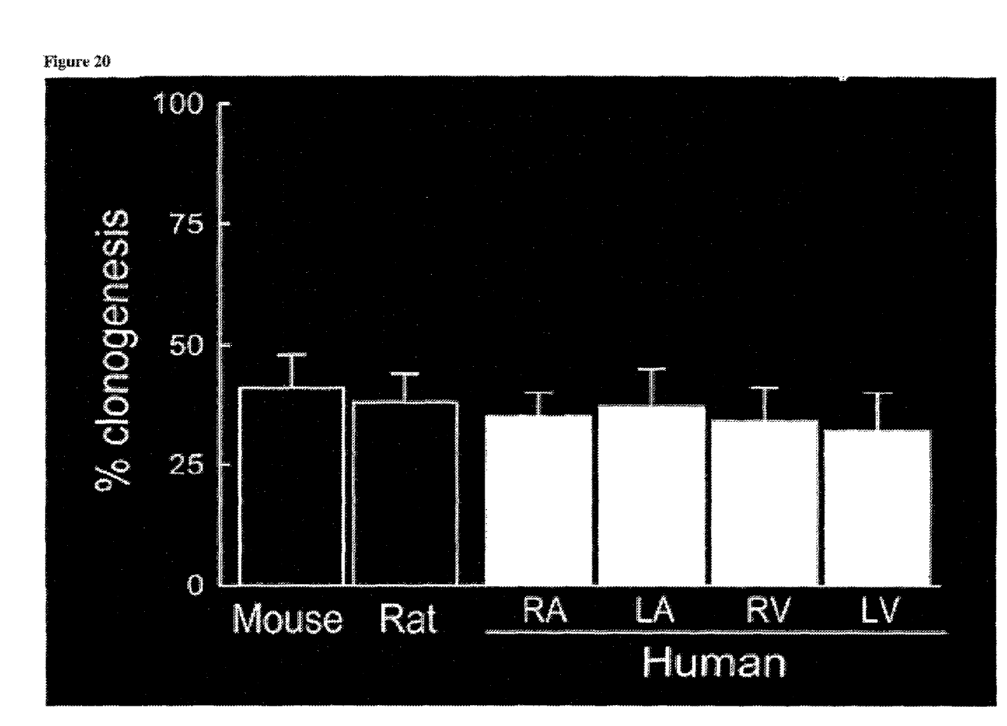

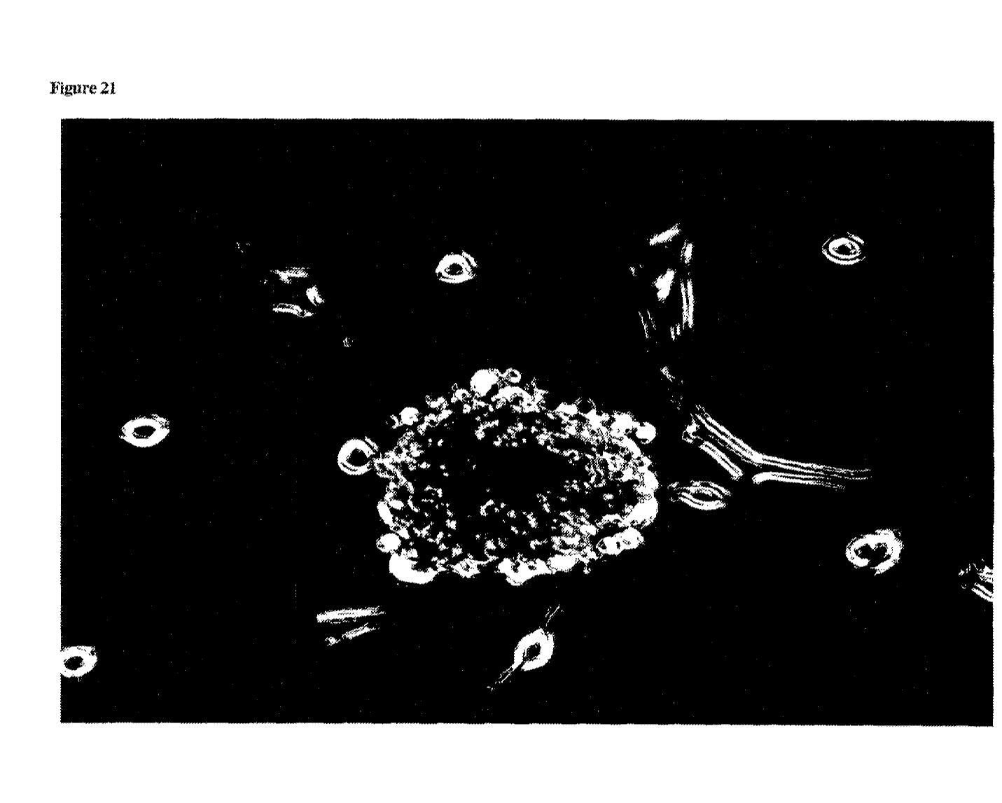

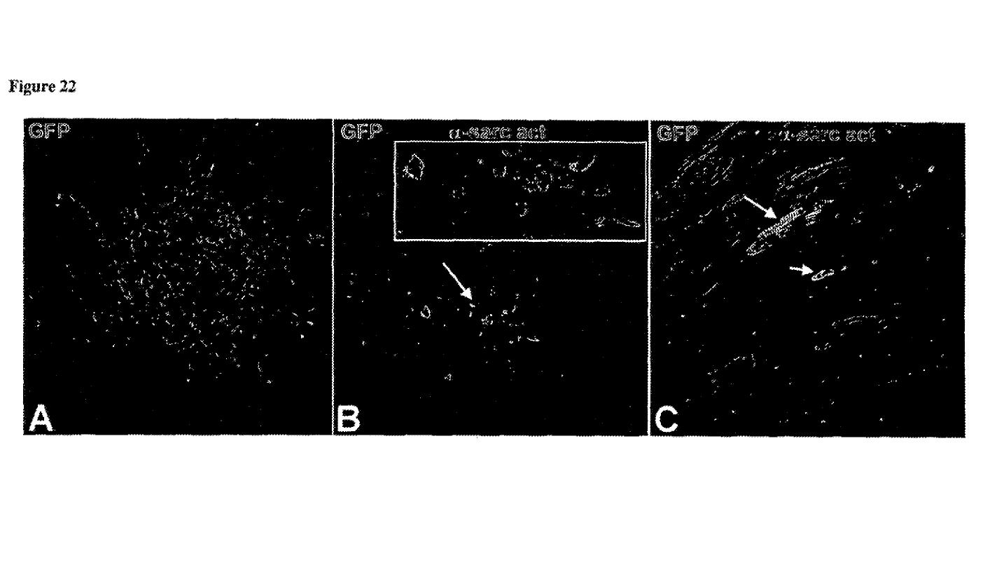

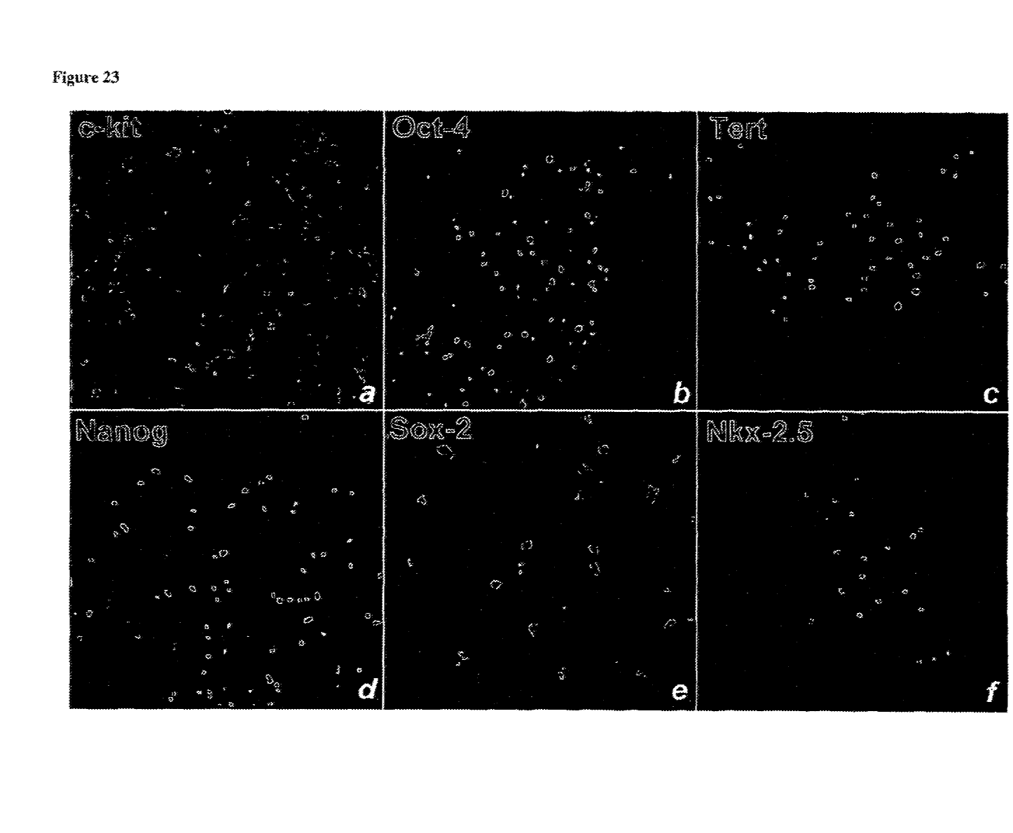

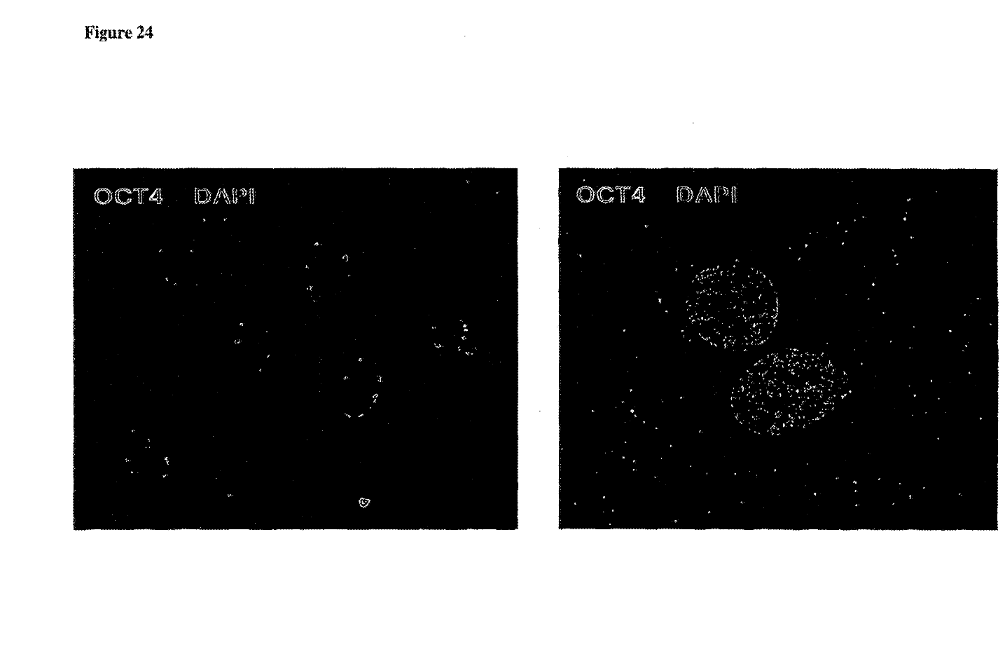

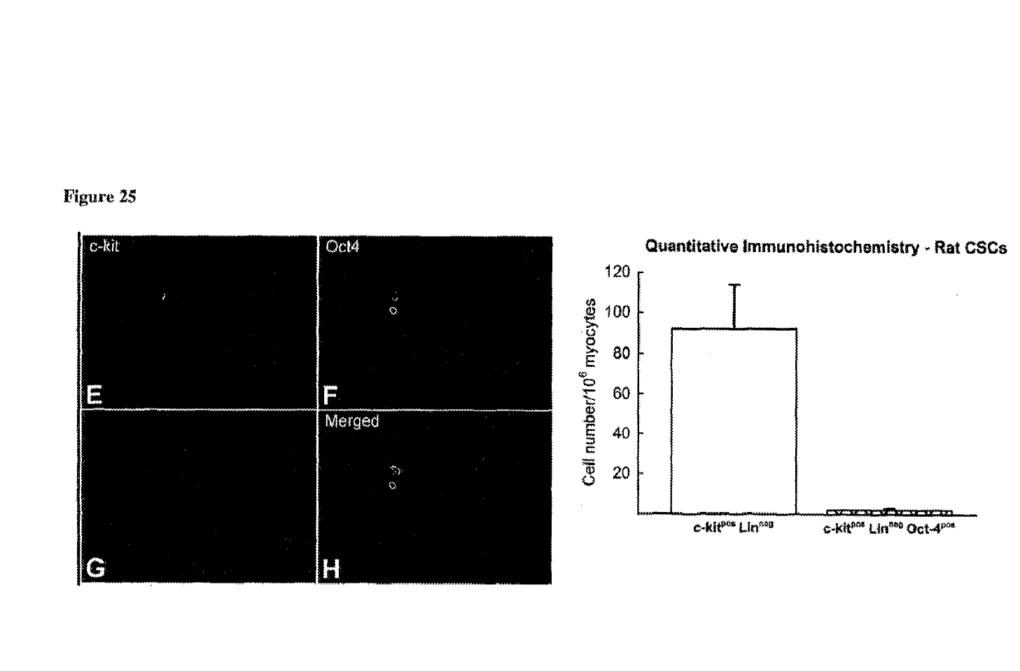

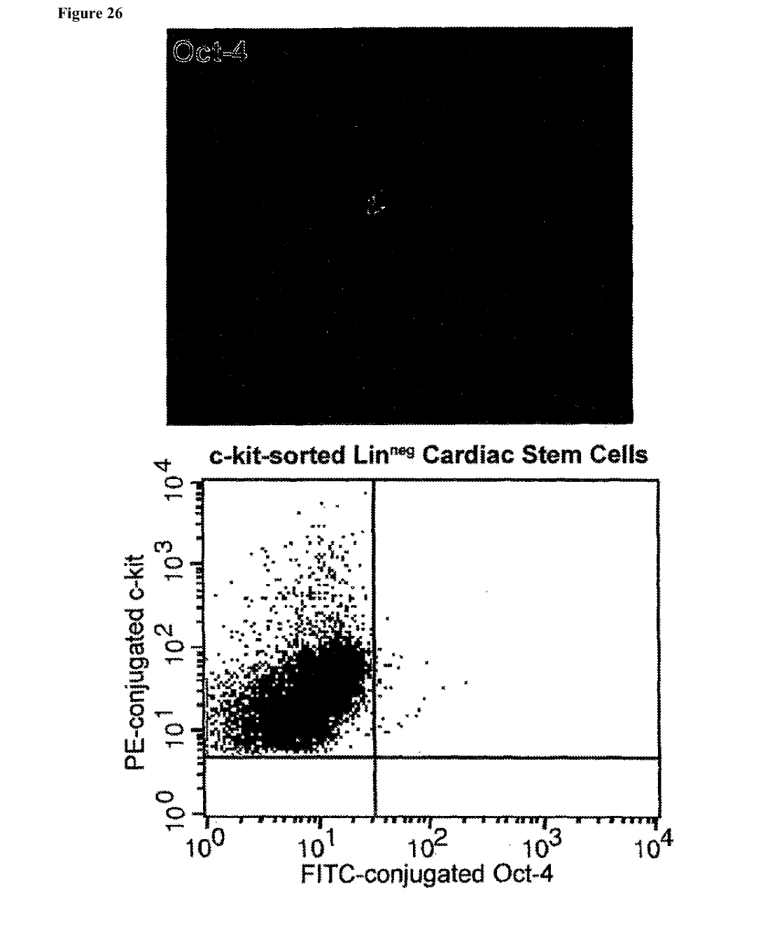

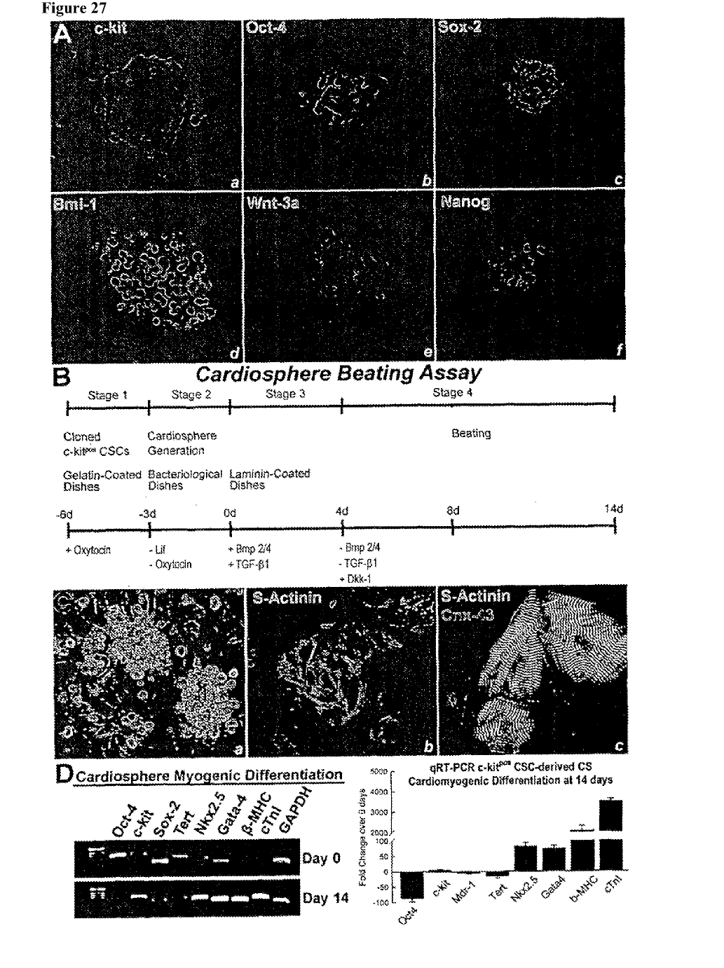

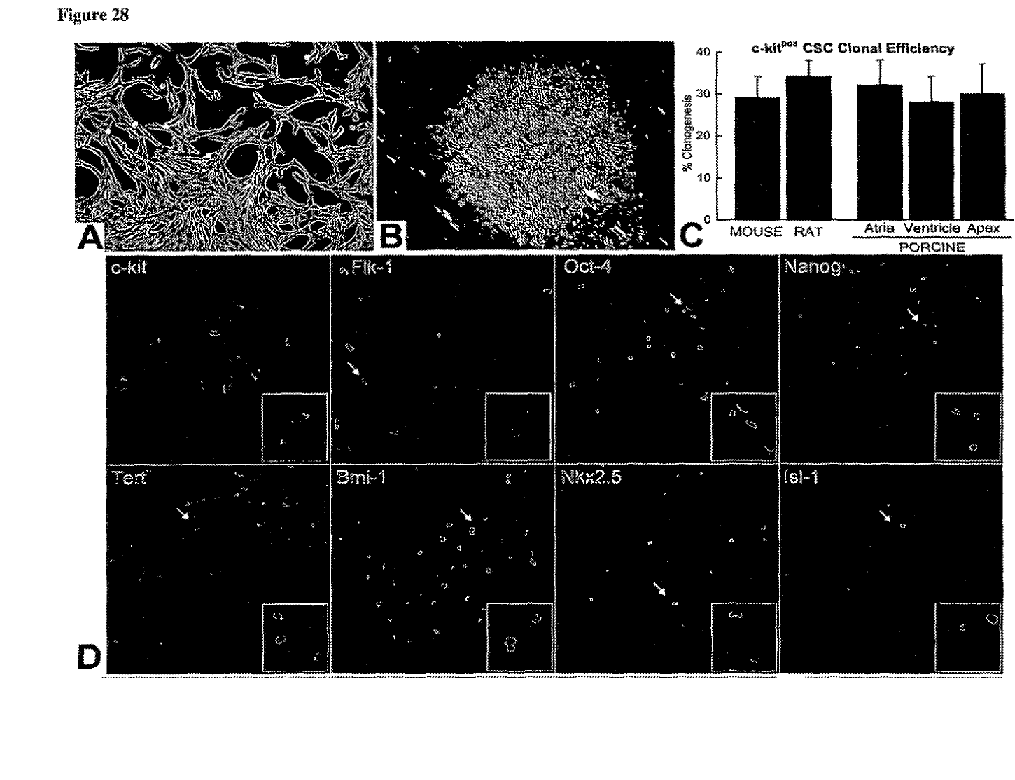

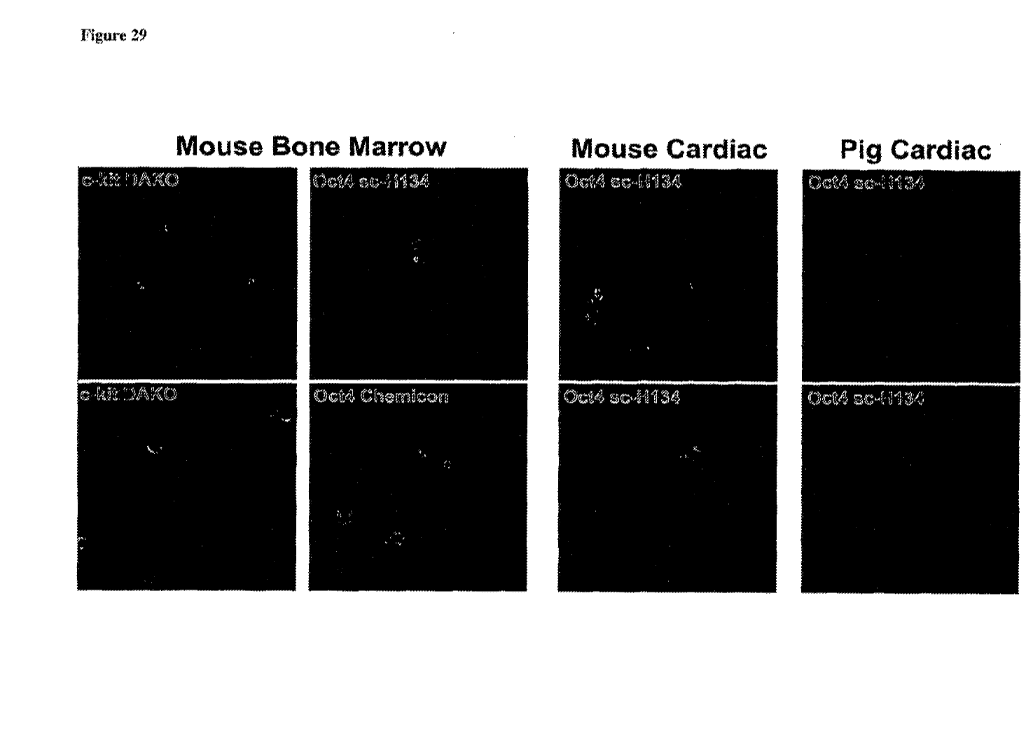

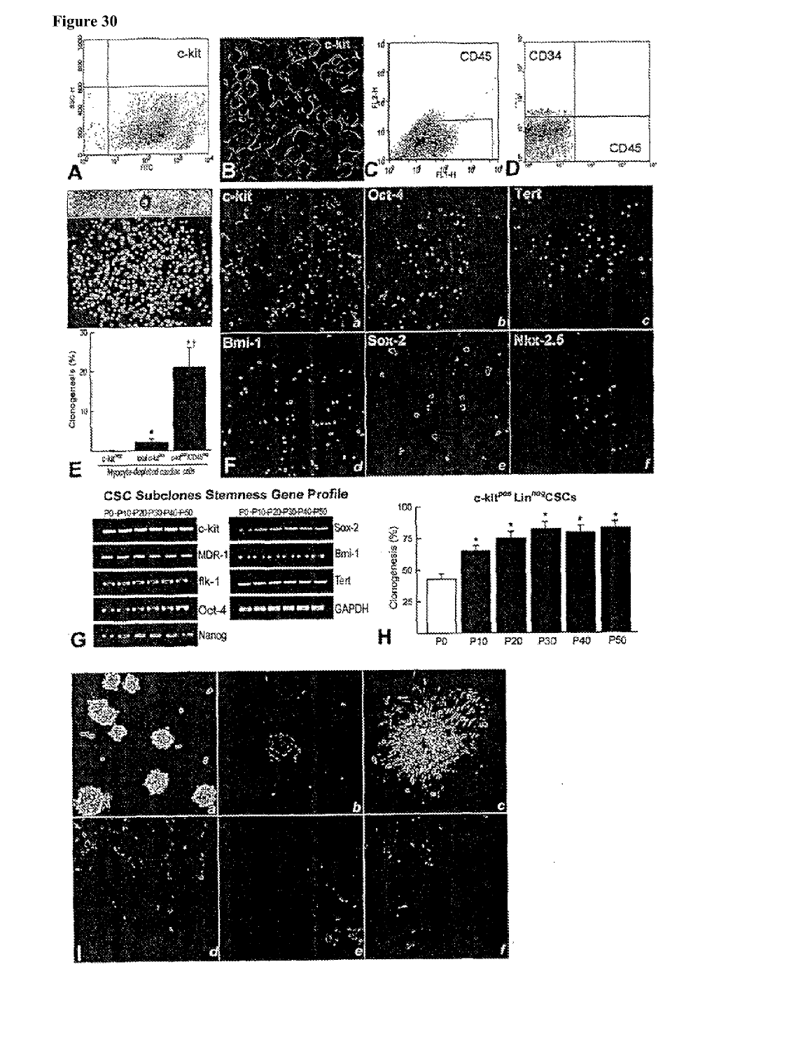

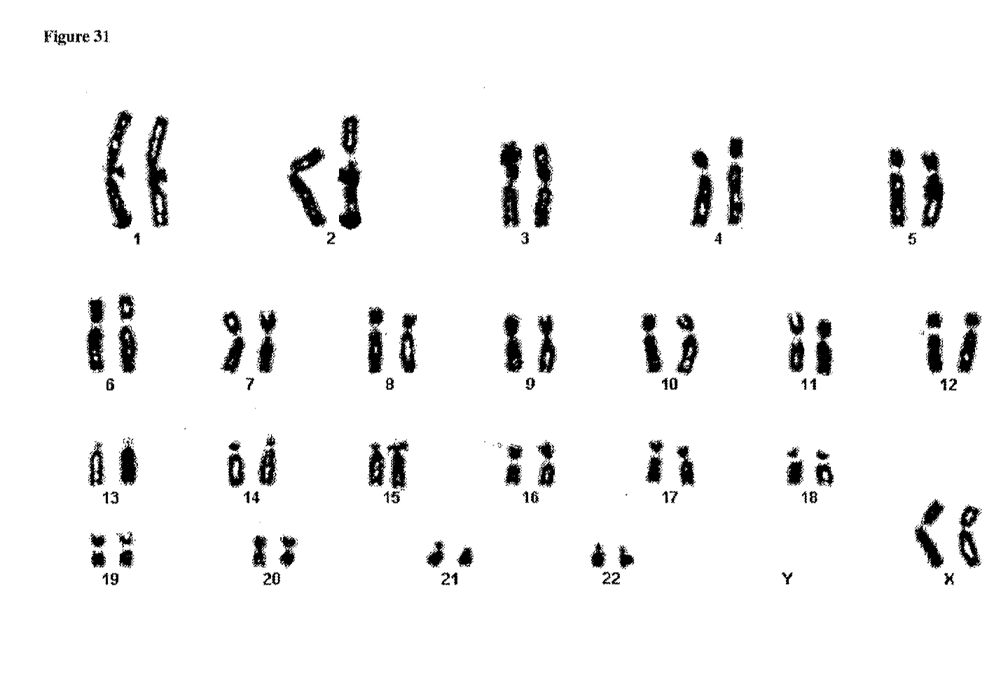

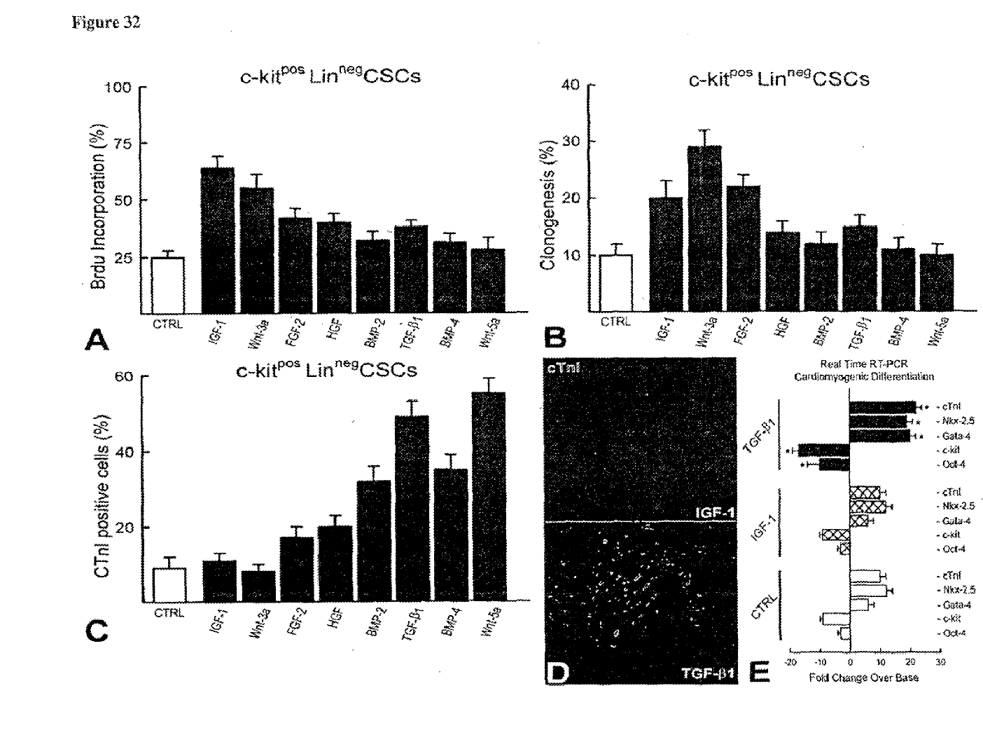

Because so far only the c-kit^{pos} cell population from the adult heart has been shown to be self-renewing, clonogenic and multipotent, c-kit^{pos} cells from the hearts of 8 week old C57BL/6 mice (n=10) were isolated by FACS and expanded in vitro. The c-kit^{pos} cells consistently represented 4-5% of the small cells fraction and, on average, 10⁵ c-kit^{pos} cells were obtained from each adult mouse heart. The purity of the sorted samples was approximately 95% (FIG. 1). Immunocytochemistry studies showed expression of Oct4 in ˜10% of c-kit^{pos} cells (FIG. 2), in agreement with the Q-PCR analysis (FIG. 1).

In order to unambiguously detect Oct4^{pos} cells in the mouse myocardium, a transgenic mouse line expressing EGFP under the control of the Oct4 promoter (Oct4-EGFP) was used. Immunofluorescence for EGFP confirmed the presence of Oct4-EGFP^{pos} cells in the newborn, young (2 weeks), adult (2 months) and senescent (24 months) mouse myocardium, identified by clear, strong signals, well above the intrinsic auto-fluorescence level of the cardiomyocytes (FIG. 2). This finding was confirmed by non-fluorescent immunohistochemistry (n=10; FIG. 3), showing that Oct4-EGFP^{pos} cells are present throughout the atrial and ventricular myocardium but are more abundant in the outflow tract region. This distribution does not change with age, but the abundance of the cells decreases from birth (483±108 cells/mm³) and youth (558±121 cells/mm³) to adulthood (112±12 cells/mm³) and senescence (33±2 cells/mm³; FIGS. 2 and 3).

A similar density of c-kit^{pos} cells expressing Oct4 has been found in the normal adult myocardium of rat, pig and human where ˜1 out of every 10-20 c-kit^{pos}, Lin^{neg}, both in situ and in vitro freshly after isolation (FIGS. 25 and 26), are positive for Oct4 and the other major multipotency genes.

### Example 9

### C-Kit^{pos}Oct4^{pos} Cells are Clonogenic and Self-Renew in Long-Term Culture

Because the c-kit^{pos} cell population isolated from the adult heart is heterogenous, comprising primitive Oct4 cells and also more committed precursors, as described above, in order to obtain pure populations of Oct4 expressing cells it is necessary to either select for expressing clones or to use some of the selection/purification methods described above.

A clonal analysis is described in detail. To better track the cells when transplanted in vivo later on, Oct4-EGFP mice were bred to ROSA26 mice (carrying the LacZ reporter gene). Freshly isolated c-kit^{pos} cells from LacZ/Oct4-EGFP mice were sorted by FACS and the clonal progeny of purified cells was expanded. The fastest growing clones (n=5) were characterized by Q-PCR. Two of them showed expression of Oct4, Nanog, c-myc, Klf4, Rex-1, FoxD3, c-kit, Bmi-1 (as well as the other Polycomb genes Mel-18, M33 and Mph1/Rae-28), BMP-2, BMP-4, Wnt-3A, Wnt-4, Wnt-11, Sca-1, c-kit, the ATP-binding cassette ABCB1/MDR-1, the stromal cell-derived factor 1 (SDF-1/CXCL12) and its receptor CXCR4, the signal transducer and activator of transcription 3 (STAT3), the LIM homeobox gene Isl-1, the leukocyte common antigen (CD45), CD 34, the receptor for the vascular endothelial growth factor (VEGF) Flk-1/KDR, the Notch pathway-related genes Notch-1, Delta-1 and Numb, and the telomerase gene (TERT) by Q-PCR (FIG. 4).

An Oct4 clone was selected for long-term propagation and differentiation in vitro and in vivo. After >130 population doublings, the cells maintained expression of the multipotency markers, although initial differentiation towards the cardiac lineages in some of the cells in the culture has been observed, as evidenced by expression of the myosin heavy chain gene MHC, the transcription factors MEF2C, GATA-4 and NRx2.5, as well as the atrial natriuretic factor (ANF), atrial and ventricular myosin light chains genes MCL2a, MCL2v and neural crest specific genes, like Musashi-1 (Msi-1), neurotropin receptor P75, Pax3, protein zero (P0) and Nestin (FIG. 4).

### Example 10

### In Vitro Differentiation of Clonal Adult Murine Cardiac c-Kit^{pos} Oct4^{pos} Cells

To induce in vitro differentiation, embryoid bodies were formed and cultured with differentiation media. Cells derived from a single cardiac murine clone differentiated into the cardiomyogenic, smooth muscle, endothelial and neural lineages, as revealed by amplification of lineage-restricted transcripts by Q-PCR: natriuretic peptide precursor type A (ANF), cardiac transcription factors Gata4, Mef2c and NRx2.5, myogenin (Myf4), smooth muscle actin (α-SMA), muscle marker desmin, von Willebrand factor (Vwf), platelet/endothelial cell adhesion molecule-1 (CD31/Pecam-1), vascular endothelial cadherin (VE-Cadherin), neural specific enolase 2 (Eno2), glial fibrillary acidic protein (GFAP), α-synuclein (Snca) and oligodendrocyte-specific cyclic nucleotide phosphodiesterase (CNP1). In all cases, lineage commitment was accompanied by downregulation of Oct-4 expression (FIG. 5). A dramatic phenotypic change was observed after in vitro neural differentiation. Neurofilament-H^{pos} cells with neural-like projections resembling neurons were detected inside the embryoid bodies and migrating on the slide, surrounded by glial-like, GFAP^{pos} cells (FIG. 5).

### Example 11

### In Vitro Differentiation of c-Kit^{pos}Oct4^{pos} Cells into Endodermal, Mesodermal and Ectodermal Cell Lineages

For differentiation into ectodermal, mesodermal and endodermal lineages, cardiac Oct4^{pos} cells were cultivated as embryoid bodies (EBs) by the hanging drop method in differentiation medium (DM). Two DM were tested for their capacity to induce differentiation. DM I contained 20% FCS, 2 mM L-glutamine, 1×MEM non-essential aminoacids, 0.1 mM 13-ME, 50 μg/ml gentamycin, 0.1 mg/ml streptomycin, 100 U/ml penicillin (Sigma), 250 ng/ml amphotericin B, 205 ng/ml sodium deoxycholate (Fungizone, Invitrogen) in Dulbecco's medium (DMEM, Gibco), supplemented with specific differentiating factors (see below). DM II contained 15% DCC-treated (like in propagation medium H, see Supplementary Methods) FBS, 1×ITS supplement (Invitrogen), 2 mM L-glutamine, 1×MEM non-essential aminoacids, 0.1 mM (3-ME, 50 μg/ml gentamycin, 0.1 mg/ml streptomycin, 100 U/ml penicillin (Sigma), 250 ng/ml amphotericin B, 205 ng/ml sodium deoxycholate (Fungizone, Invitrogen) in Dulbecco's medium (DMEM, Gibco), supplemented with specific differentiating factors (see below). For this purpose 20 μl drops of differentiation medium containing cardiac Oct4^{pos} cells (n=80) were placed on the lids of bacteriological Petri dishes filled with PBS containing 50 μg/ml gentamycin, 0.1 mg/ml streptomycin, 100 U/ml penicillin (Sigma), 250 ng/ml amphotericin B, 205 ng/ml sodium deoxycholate (Fungizone, Invitrogen) and cultured in hanging drops for 2 days and in bacteriological petri dishes for 3 days. At day 5, EBs were transferred to gelatine-coated dishes. To induce neural differentiation, DM I/II also contained 100 ng/ml FGFb, 20 ng/ml EGF (Peprotech) and 1×B27 supplement with vitamin A (Invitrogen). The endothelial DM I/II was supplemented with 10⁻⁸ dexamethasone (Sigma) and 10 ng/ml vascular endothelial growth factor (VEGF, Peprotech). To trigger differentiation into the smooth muscle lineage, DM I/II also contained 50 ng/ml platelet-derived growth factor-BB (PDGF-BB, Peprotech). For cardiomyogenic lineage differentiation, different combinations of reagents including 1% dimethyl sulfoxide (DMSO), 10 μM 5-azacytidine, 10 μM oxytocin, 10⁻⁸ M retinoic acid, 0.1 mM ascorbic acid (Sigma), 29 nM FGFb, 2.5 ng/ml transforming growth factor beta-1 (TGFβ1), 4 nM cardiotrophin-1 (Peprotech), 40 nM thrombin (Sigma) were tested in DM I/II.

### Example 12

### In Vivo Differentiation of Freshly Isolated Adult Murine Cardiac c-kit^{pos} Oct4^{pos} Cells

To assess whether the multipotency gene expression profile of c-kit^{pos} Oct-4^{pos} cells correlates with an unsuspected in vivo developmental potential, the cells' ability to integrate into the early embryo environment and participate in the formation of different tissues was tested.

To this end, freshly fertilized chicken eggs (White Leghorn) were incubated at 38° C. 300 chicken embryos at E11-13 stage (Hamburger&Hamilton) were exposed after opening the shell and removing the overlaying chorion and injected into the amniotic cavity with 10⁵ (n=200) or 10⁶ (n=100) cardiac c-kit^{pos} cells obtained from Oct4-EGFP/Rosa26 mice using a microsyringe (Hamilton). The shells were sealed with Parafilm® and the eggs incubated for 5 days at 38° C. The embryos were collected and briefly washed in PBS at 4° C. before extracting the genomic DNA.

PCR analysis showed that 7 out of 18 surviving embryos (39%) were chimeric in their head and trunk regions after amplification of the LacZ and the EGFP transgenes from the genomic DNA (FIG. 6) and identification of donor-derived parenchymal cells in different tissues of the injected chicken embryos (data not shown).

To better characterize this putative developmental potential, 10-15 cardiac c-kit^{pos} cells freshly isolated from LacZ/Oct4-EGFP mice, containing on average a single Oct-4^{pos} cell (see above), were injected into 3.5 dpc wild type mouse blastocysts. Wild-type blastocysts (3.5 dpc) were collected from the uteri of superovulated females by flushing with HTF medium (Specialty Media). Superovulation was induced by injection of 7.5 IU pregnant mare serum gonadotropin followed by injection of 5.0 IU human chorionic gonadotropin after a 48-hour interval. The collected blastocysts were washed with and cultivated in KSOM (Specialty Media) under 5% CO₂ in air at 37° C. Blastocyst injection was carried out by injecting 10-15 cells using standard procedures.

The injected embryos were transferred to foster mothers and allowed to develop until 13.5 dpc. At this stage the embryos were collected, briefly washed in PBS with 2 mM MgCl₂ at 4° C. and fixed for 3 h at 4° C. under agitation with freshly prepared 2% paraformaldehyde, 0.2% glutaraldehyde, 5 mM EGTA, 2 mM MgC12, pH 7.4. Conventional X-gal staining was performed for 24 h at 37° C. Embryos were cryopreserved using the isopentane method and 10 μm sections were obtained using a cryostat. Chimerism in adult mice was first assessed by PCR analysis of genomic DNA obtained from tail biopsies, using primers specific for the LacZ gene.

To amplify the transgene, DNA was extracted using the QIAamp® DNA Mini Kit (Qiagen) following the manufacturer's recommendations, with the exception that the elution step was performed in 3 steps. In each step 40 μl elution buffer, pre-warmed at 70° C., was added to the column and a 3 min lapse was applied before centrifuging to facilitate DNA dissolution and recovery. 1 μg of genomic DNA was used for amplification. The PCR mix contained 1 mM MgCl₂, 0.2 mM dNTPs, 0.5 μM primers and was prepared on ice before incubating 5 min at 94° C. (hot start) in the iCycler PCR machine (BioRad) to increase specificity. Forty PCR cycles of 94° C. (30 s), 60° C. (30 s) and 72° C. (30 s) were followed by 10 min elongation at 72° C.

Animals from whose genomic DNA the LacZ gene could be amplified were sacrificed 1-3 months after birth. In order to check for chimerism in adult tissues, animals were heparinized under anesthesia and perfused through the left ventricle with 15 ml of PBS with 2 mM MgCb at 4° C. and 120 ml of freshly prepared 2% paraformaldehyde, 0.2% glutaraldehyde, 5 mM EGTA, 2 mM MgCl₂, pH 7.4. The organs used for analysis were excised and further fixed in the same fixative for 2.5 h at 4° C. under agitation. After 3 washes (30 min) in PBS with 2 mM MgCl₂ at 4° C. under agitation, the organs were cryoprotected, embedded and frozen-sectioned as described above.

Tissue sections were washed twice (5 min) in PBS with 2 mM MgCl₂ and two times (10 min) in X-gal basic buffer: 2 mM MgCl₂, 5 mM EGTA, 0.01% sodium deoxycholate (Sigma), 0.02% Nonidet P-40 (Roche) in 500 ml PBS, pH 7.4. The sections were incubated 12-16 h at 37° C. in a solution containing 5 mM K3(Fe(CN)6), 5 mMK4(Fe(CN)₆) (Sigma) and 1 mg/ml X-gal (Biosynth). In order to assess the specificity of the staining, the X-gal reaction was followed by immunohistochemistry using antibodies against X-gal (1:100; Cappel) and a biotinylated goat anti-rabbit antibody (1:200, Pierce). The avidin/biotin blocking kit and the ABC kit (Vector Laboratories) were used, following the manufacturer's recommendations. Tissue sections were incubated for 30 min with 0.7 mg/ml 3,3′-diaminobenzidine in 60 mM Tris buffer and the peroxidase reaction was performed by incubating the sections with 0.05% H2O2 and 0.7 mg/ml 3,3′-diaminobenzidine in 60 mM Tris buffer. For immunofluorescence, primary antibodies for Lin^{pos} cells (Miltenyi Biotech, 1:100), albumin (Dako, 1:1000), Pecam-1 (Becton Dickinson, 1:100), Desmin (Dako, 1:100) were used.

The foetuses with highest and lowest chimerism according to X-gal staining are shown in FIG. 6. Immunohistochemistry for β-galactosidase confirmed the specificity of the X-gal staining. The LacZ and the EGFP transgenes were also amplified by PCR from the genomic DNA of all the embryos where the histological analysis showed chimerism (n=9; 45%), as described above. Cells of donor origin were particularly abundant in the intestine, liver and peritoneum (FIG. 6). Because the freshly isolated c-kit^{pos} population is heterogeneous and therefore multilineage differentiation could rely on the presence of different progenitors in fresh isolates, a more detailed study was performed using cells derived from a single clone in order to test whether they proved to be multipotential in vivo.

### Example 13

### In Vitro Differentiation of C-Kit^{pos} Oct4^{pos} into Biochemically, Morphologically and Functionally Well Differentiated Spontaneously Beating Cardiac Myocytes

Although CSCs increased transcription of cardiomyocyte lineage-specific genes and expression of cardiomyocyte proteins after supplementation either spontaneously or after supplementation of a differentiation medium with Wnt5a, TGFβ-1, BMP-4 or BMP-2, the resulting myocytes remained immature, without organized sarcomeres and never reached a contractile, fully functional phenotype.

To unambiguously substantiate a precursor-product relationship between cloned c-kit^{pos} Oct4^{pos} CSCs and fully differentiated beating myocytes it is necessary to show specialisation of individual or cloned CSCs into functional, contracting cardiomyocytes and to identify the molecule(s) responsible for inducing this phenotype. Therefore, we tested different factors, either by themselves or in combination with other factors, for their ability to produce spontaneously contractile cardiomyocytes derived from cloned c-kit^{pos} Oct4^{pos} CSCs.

Oxytocin, a mammalian hormone best known for its roles in female reproduction, has been shown to play a key role in myogenic differentiation of stem cells (Oyama et al. 2007; Matsuura et al. 2004 and others from BM and ESCs), although its mechanism of action is still undefined. Adherent cloned c-kit^{pos} CSCs were treated with 100 nM Oxytocin for 72 hours (Oyama et al. 2007; Matsuura et al. 2004) before the generation of cardiospheres. In agreement with their primitive state, these c-kit^{pos} CSC-derived cardiospheres expressed stemness markers, such as Oct-4, Sox-2, Bmi-1, Nanog, Klf-4 as well as early commitment to the cardiomyocyte lineage by expressing NRx2.5 (FIG. 27A).

The cardiospheres were transferred to laminin-coated plastic dishes with a myo-cardiogenic medium consisting of α-MEM (base medium), supplemented with 2% FBS, dexamethasone (1 μM), ascorbic acid (50 μg/ml), β-glycerophosphate (10 mM), TGF-β1 (5 ng/ml), BMP2 (10 ng/ml), and BMP4 (10 ng/ml) (FIG. 27B). After 4 days, TGF-31, BMP2, and BMP4 were removed from the media. For the remaining 10 days, the media was supplemented with the canonical Wnt inhibitor, Dickkopf-1 (DKK-1; 150 ng/ml) (FIG. 27B). At day 8, immunostaining for sarcomeric actinin (SA) showed that the cells within the cardiosphere had differentiated into cardiomyocytes with well organized and abundant sarcomeric structures. These myocytes were connected to one another through connexin 43 gap junction-looking structures (FIG. 27C). Furthermore, cardiospheres had initiated rhythmic beating at day 8 which was maintained for the duration of the culture. A beating phenotype was also exhibited by isolated cells that had migrated from the sphere and were attached on the periphery as an adherent cell layer. qRT-PCR at different time points of CSC culture in the cardiomyogenic differentiation cocktail, showed a progressive decrease in transcripts for stemness and concomitant up regulation of genes specific for the cardiomyocyte lineage (FIG. 27D-E).

These findings unambiguously document the generation of bona fide autonomously beating myocytes by cloned c-kit^{pos} CSCs. In addition, they provide an in vitro cardiomyocyte beating differentiation assay which has not been previously available. This assay, obviates the need for co-culture of the precursor cells with neonatal or adult cardiomyocytes.

### Example 14

### A Single Adult Murine Cardiac c-kit^{pos} Oct4^{pos} Clone Contributes to the Formation of Tissues Derived from Different Germ Layers

To determine whether the progeny of a single cardiac Oct4 cell could give rise to different adult cell lineages when placed in the early murine embryo, 10-15 cells from the LacZ^{pos}/Oct4-EGFP^{pos} clone were injected into 3.5 dpc wild type blastocysts and the foetuses were allowed to develop to term. PCR analysis from genomic DNA extracted from tail biopsies showed chimerism in 26 out of 71 pups (37%). The animals were sacrificed at 1-3 months. Histological analysis (FIG. 8) revealed the presence of cells stained by both X-gal and anti-β-galactosidase antibodies in reproductive organs (6%, n=4), heart (6%, n=4), brain (10%, n=7), kidney (4%, n=3), spleen (6%, n=4), skin (8%, n=6), liver (10%, n=7), lung (6%, n=4), skeletal muscle (6%, n=4), bone marrow (7%, n=5) and intestine (4%, n=3). Localization of X-gal deposits in cells displaying cell type-specific markers confirmed the differentiation of the cloned LacZ^{pos}/Oct4-EGFP^{pos} cells into lineages derived from different germ layers: albumin-containing hepatocytes, lineage markers-expressing (Lin^{pos}) cells in the spleen and the bone marrow and desmin negative satellite cells displaying their characteristic morphology in the surface of the skeletal muscle (FIG. 8).

Similar to mouse CSCs, rat adult stem cells according to the invention also have the potential to differentiate into various tissues. When induced to differentiate in vitro these cells initially form pseudo-embryoid bodies and later differentiate into various tissues (FIG. 7).

In summary, the inventors have shown that the cells of the invention have the potential to develop into tissues derived from all three germ layers. This is in contrast to previously described adult stem cells, which can only differentiate into the tissue from which they were derived.

### Porcine Adult Stem Cells

### Example 15

### Isolation and Culture of Porcine Cardiac Stem Cells

A Young Yorkshire-Albino pig (25 kg) was sedated with an intramuscular injection of ketamine (30 mg/kg), heparinized and euthanized with pentobarbital sodium. Following thoracotocmy, the thoracic aorta was cannulated with a 6.35 mm I.D. Tygon® tube (Scientific Commodities, Inc.) and the cannula was advanced into the ascending aorta until the tip was located just distal to the aortic valve. The cannula was tightly tied to the aorta using umbilical tape (Fisher), and the heart was immersed in a beaker containing perfusion solution at 37° C. and bubbled with 100% O₂. The heart was washed by retrograde perfusion through the coronary circulation, at 100 ml/min flow rate, with 2000 ml buffer containing: 125 mM NaCl, 30 mM HEPES, 1.2 mM KH₂PO₄, 4.75 mM KCl, 1.2 mM MgSO₄; 3.9 g dextrose and 1 U/ml heparin in low potassium Krebs-Henseleit buffer (Sigma), pH 7.5. The heart was further washed with 1000 ml of the same buffer, without heparin, and then perfused, at the same rate, with a similar buffer containing also 0.1% BSA, 1×BME vitamins solution, 1×MEM non-essential amino acids, 24.9 mM creatine, 58.5 mM taurine, 3 mM L-glutamine and 25 μM EGTA (Sigma). After 1 min, 75 U/ml collagenase type II (Worthington) was added, and the perfusion continued until the heart started to show signs of digestion (45 min). At this moment the heart was detached from the perfusion system and divided with a sterile knife into three parts (atrium, ventricle and apex) that were processed separately using the same procedures described above for the isolation of murine cardiac c-kit^{pos} cells. For clonal studies, four 96-well gelatine-coated plates (Becton Dickinson) were seeded for each region of the heart. Medium IV was used for the expansion of porcine cardiac c-kit^{pos} cells, with the only difference that human LIF (Chemicon) was used instead of the murine one.

c-kit^{pos} porcine cardiac cells were obtained by magnetic immunobead sorting and the purity of the preparation assessed by flow cytometry. Cell surface antigen staining was performed at 4° C. for 30 minutes using fluorochrome conjugated monoclonal rat anti-mouse antibodies reactive to c-kit (all from Pharmingen). All c-kit positive cells were negative for CD34 and CD45, and the cells expressing either of these two antigens were eliminated from the population. Respective isotype controls (Pharmingen) were used as negative controls. Propidium iodide (P1) (2 μg/mL) was added before fluorescence-activated cell sorting (FACS) to exclude dead cells.

### Example 16

### Porcine c-Kit^{pos} Cells are Clonogenic, Self-Renewing and Pluripotent

c-kit^{pos}, CD34^{neg} and CD45^{neg} cells were plated for 7-10 days at 2×10⁴ cells/ml in F12K medium containing 10% FCS, bFGF, EPO and LIF. After recovery, they were moved to modified neural stem cell medium (mNSCM): Dulbecco's MEM and Ham's F12 (ratio 1:1), bFGF (10 ng/ml), EGF (20 ng/ml), LIF (10 ng/ml), EPO and insulin-transferrin-selenite. To show clonogenicity of these cells, single cell cloning was employed. Isolated c-kit^{pos} cells were collected with Miltenyi immunomagnetic microbeads. Before sorting, bead-coated cells were treated first in enriched F12K medium and then in mNSCM for 15 days. Subsequently, ˜20,000 cells were sorted (MoFlo High Performance Cell Sorter, Cytomation), and single cells were deposited in Terasaki plates. The individual cells were grown in F12K medium for 1-2 weeks when clones were identified and expanded. Aliquots of each clone were tested for expression of Oct-4 at the mRNA and protein levels (FIG. 28).

The progeny of a single cell clone can be expanded through hundreds of passages for several years without the appearance of detectable chromosomal abnormalities or loss of the growth and differentiation properties of the cells. FIG. 9 shows a karyotype of a porcine CSC which had been cultured for 2.5 years.

### Example 17

### The c-kit^{pos} Oct4^{pos} Cells Derived from Adult Tissues have a High Tropism for their Tissue of Origin when Administered into the Systemic Circulation

To determine whether the c-kit^{pos} Oct4^{pos} cells originated from the myocardium had a tropism for their tissue of origin when this tissue has been damaged and presumably secretes homing factors, we tested whether myocardial injury facilitates the homing of transplanted c-kit^{pos} Oct4 CSCs and their ensuing differentiation into new myocytes. We injected 5×10⁵ cloned rat c-kit^{pos} CSCs, genetically tagged with a GFP expressing vector, through the tail vein of 5 rats 12 hours after myocardial injury. It should be noted that these cells have been passaged in culture >50 times and had also been subcloned twice. Therefore, whatever homing behaviour was exhibited could not be due to the presence of “homing molecules” originated from the tissue still present on the cells.

Two types of controls were carried out in parallel: as a cell control, we injected c-kit^{neg}, myocyte-depleted cardiac cells (MDCCs, 5×10⁵), similarly genetically tagged, to an additional 5 myocardial-injured rats; as a control for the role of the injury in the homing, both cell types were similarly administered to the same number of uninjured control animals. The presence of transplanted CSCs and/or their progeny in the heart and other tissues was determined 6 and 28 days later.

Within the hearts of CTRL, and myocardial-damaged rats transplanted with GFP^{pos} c-kit^{neg} MDCCs, on average there were <1 GFP^{pos} cell per 3×10⁴ nuclei at 6 and 28 days post-transplantation (FIG. 22). We also observed very few GFP^{pos} cells at either time point (≦1/10⁴ nuclei) in the hearts of the CTRL animals transplanted with GFP^{pos} c-kit^{pos} Oct4^{pos} CSCs. Importantly, none of these GFP^{pos} cells expressed any nuclear or cytoplasmic myocyte markers at any of the times analyzed (data not shown).

In contrast to the above, in myocardium-injured hearts transplanted with GFP^{pos} c-kit^{pos} Oct4^{pos} CSCs, there were 83±11 and 26±7 GFP^{pos} cells per 10⁴ nuclei at 6 and 28 days, respectively (FIG. 22). The GFP^{pos} cells were most abundant in the more damaged myocardial layer (sub-endocardium, 43%; Mid-wall, 23%; sub-epicardium, 8% of the GFP^{pos} cells). If this sampling were representative of the whole myocardium of the ISO-injured animals, there would be ˜1.2×10⁶ GFP^{pos} cells per heart at 6 days, indicating a very efficient cardiac homing and replication of the transplanted cells once they had nested in the heart. The cycling of the transplanted cells was confirmed by the high percentage of Ki67^{pos} GFP^{pos} cells at 6 days (20±4% of GFP^{pos} cells) and 28 days (8±3% of GFP^{pos} cells) post CSC transplantation. A significant fraction of these GFP^{pos} cells co-expressed Nkx-2.5 (40±8% at 6 days; 19±5% at 28 days. We also detected an increase in GFP^{pos} cells which expressed CTnI at 6 days (25±3%) and 28 days (42±3) (FIG. 7F-G). At 28 days, there were also increased number of larger and more differentiated GFP^{pos}/CTnI^{pos} cells (4±1%) which in general were in close contact with spared myocytes (FIG. 22).

At 6 days post injection, GFP^{pos} cells were found in all extra-cardiac tissues examined (lung, spleen, liver, skeletal muscle). The highest GFP^{pos} cell count was observed in the lung and spleen, but in each case their number was in the range of a few GFP^{pos} cells per 10⁴ nuclei and, therefore, significantly below the levels detected in the myocardium. At 28 days the GFP^{pos} cells had disappeared from all extra-cardiac tissues, except in skeletal muscle where a few isolated GFP^{pos} cells were still identified, a not surprising finding because skeletal muscle tissue is also damaged by the myocardial insult (see Goldspink et al. 2004). None of the GFP^{pos} cells identified in extra-cardiac tissues expressed nuclear or cytoplasmic cardiomyocyte markers.

These findings suggest that the CSCs have a strong tropism for the damaged myocardium, where the cardiopoietic factors secreted by the surviving (stressed) myocytes serve as positive chemotactic agents, since no such homing occurs to healthy myocardium or by cells which do not express the corresponding membrane receptors. These data also show that the injured myocardium provides a homing milieu which not only attracts circulating CSCs but also foster their survival, stimulates self-renewal as well as their activation and differentiation into new cardiomyocytes.

### Example 18

### Heterologous Porcine Cardiac Oct 4 Cells Efficiently Induce Activation, Multiplication and Differentiation of Resident Stem Cells Resulting in Regeneration of the Myocytes and Microvasculature after Infarct

The cloned porcine myocardial-derived Oct4 positive cells described above were tested for their capacity to induce myocardial regeneration in the pig. The pig was chosen as a model system due to the similarities to humans regarding heart size, poor collaterals, and physiology. The cells had been isolated from a male White York pig from an American strain three years before being transplanted into White York pigs from Spanish strain. These two strains are unrelated and, therefore, immunologically very different.

Before the production of the myocardial infarction, the animals were treated for 3 days with 81 mg of aspirin, 71 mg of Plavix and Labetalol. This anti-plaquete and anti-coagulatory therapy was maintained for a whole week after the production of the infarct. After sedation with 100 mg of Telazol 1M, the animals were anesthetized with isofluran (2-5% in O₂) whose concentration was adjusted as needed.

Once the animal was fully anesthetized, a catheter sheet French size 4.5-6 was introduced into the left femoral artery and 70 units of heparin were administered through this route. A guide catheter 6French AR-1 or AR-2 was introduced into the left coronary artery through the coronary ostium and the vessel was visualized by means of radiographic contrast. The coronary left anterior descending below the emergence of the first septal artery was identified because its occlusion produces an infarct anteroseptal or anteroapical with extension to the interventricular septum. At this point 2 mg/kg of Lidocaine are administered and the balloon of 2.3-3 mm diameter and 15 mm length at the tip of a catheter (143 cm) was inflated and maintained in position for 90 min. Special care was taken not to occlude the first septal artery as this almost always results in mortality. After 90 min of balloon occlusion, the balloon was deflated, a new angiopraphy was performed and the cell preparation (1-2×10⁹ cells expressing high levels of GFP) in 15 ml of porcine serum at body temperature was administered. For the controls, porcine serum without addition of cells was administered. After the cells were transplanted, the balloon was removed and the femoral artery was repaired. An angiography together with a 2D echocardiography was performed and the animal's blood pressure was taken.

Blood samples taken from the systemic circulation during the cell administration, at 12 and 24 hours after did not detect GFP positive cells in the systemic blood. Moreover, blood samples taken from the coronary sinus every 10 min. during the cell administration and up to one hour after the procedure also failed to detect more than a few occasional GFP cells, indicating that the vast majority of the injected cells had remained in the myocardium. This hypothesis was corroborated with the analysis of three animals sacrificed at 4, 24 and 48 hours after the cell infusion. None of these three animals had GFP positive cells in any of the organ analysed (lung, spleen, liver, skeletal muscle). Moreover, planimetric analyses of myocardial sections and quantification of the GFP cell concentration accounted for all the cells administered being present in the myocardium, within the broad margin of error of these techniques.

Three weeks after myocardial infarction, the animals were sacrificed and the heart together with samples of other organs was tested for the presence of the transplanted cells.

It was found that the ventricular function of the treated animals improved ˜15% in ejection fraction and velocity of shortening as compared to the controls. Furthermore, the size of the scar in the treated animals was about 10% smaller compared to the controls.

No transplanted cells were found either in the myocardium or any of the tissues analysed in any of the animals, while in a group of animals sacrificed 24 hours after the myocardial infarction and cell transplant, between 95-100% of the transplanted cells were found into the infarcted areas of the myocardium with a small number in the neighbouring myocardium. No cells were found in any of the tissues tested: liver, lung and spleen.

Immunohistology of the control and treated hearts showed a reduction of collagen in the scars of the treated animals and a reduced number of inflammatory cells. The scar was teeming with a large quantity of resident cells in the cell cycle and large numbers of progenitors, precursor and newly developed myocytes and capillaries. These cells could be identified because they were marked with BrdU, which was administered to the animals after infarction in order to identify all the cells born after the cell transplant. The number of stem cell/progenitors in the infarcted area of the treated animals is 5-7 times higher than in the control group. The total number of myocytes and capillaries lost by the infarct (˜2×10⁷ myocytes per gram of tissue and a similar number of endothelial cells) had fully regenerated.

### Example 19

### C-Kit^{pos} Cells Contribute to Myocardial Regeneration

c-kit^{pos}, CD34^{neg} and CD45^{neg} cells isolated from B16J male mice were plated for 7-10 days at 2×10⁴ cells/ml in F12K medium containing 10% FCS, bFGF, EPO and LIF. After recovery, they were moved to modified neural stem cell medium (mNSCM): Dulbecco's MEM and Ham's F12 (ratio 1:1), bFGF (10 ng/ml), EGF (20 ng/ml), LIF (10 ng/ml), EPO and insulin-transferrin-selenite. To show clonogenicity of these cells, single cell cloning was employed. Isolated c-kit^{pos} cells were collected with Miltenyi immunomagnetic microbeads. Before sorting, bead-coated cells were treated first in enriched F12K medium and then in mNSCM for 15 days. Subsequently, 20,000 cells were sorted (MoFlo High Performance Cell Sorter, Cytomation), and single cells were deposited in Terasaki plates. The individual cells were grown in F12K medium for 1-2 weeks when clones were identified and expanded. Aliquots of each clone were tested for expression of Oct-4 at the mRNA and protein levels. Oct-4^{pos} clonogenic cells were expanded, and aliquots grown in specific differentiation medium for myocyte, vascular smooth muscle and endothelial cell specification. Clones from 5 of these clones were transfected with a lentivirus producing GFP driven by a promiscuous promoter at a high PFU so that >90% of the cells were GFP positive.

Groups of 5 B16J female mice were produced an anterior myocardial infarct by ligation of the anterior descending coronary artery as previously described (Beltrami et al., 2003). Immediately after the production of the infarct 1×10⁵ c-kit^{pos} Oct-4^{pos} cells were injected, divided in two doses, at diametrical sides of the infarct zone in a 2 μl volume together with fluorescent polystyrene nanobeads to mark the side of injection and to determine whether the injection had been made intrmyocardically or through the wall into the ventricular cavity. Cells originated from five different clones were each injected into a group of 5 infarcted animals. One group of 5 similarly infarcted animals and treated with 2 μl of saline served as control.

Ten days after the injection all animals were sacrificed. Based on the presence of fluorescent beads within the ventricular wall it was determined that the cell injection had been successful in 76% of the animal, while into the ventricular cavity in the other 24%. All control animals as well as all those in which the cell injection had been unsuccessful had well organized scars in the anterior face of the ventricle and the apex. Histologically the infarct was transmural with a very thin layer of epicardial or endocardial muscle remaining in the area of the infarct. In contrast, 15 of the successfully cell treated animal (out of 19) showed a strong band of myocardial regeneration constitutes by immature myocytes, capillaries and arterioles with a marked decrease in collagen content as compared to the controls. The cells in this regenerated band were GFP positive and those tested by FISH were positive for the mouse Y chromosome while the surrounding GFP negative cells were negative for this marker, presumably because they were host cells and, therefore, XX as corresponds to females.

### Human Adult Stem c-Kit^{pos} Oct-4^{pos} Cells

### Example 20

### Isolation of Adult Multipotent Stem Cells from Human Myocardium

C-kit^{pos} cells were isolated from surgical biopsies obtained from the right atrium and left ventricle of human patients undergoing cardiac surgery. The cells were isolated from either the atrial or the ventricular myocardium by either one of three methods:

a) After mincing, the tissue was initially dissociated with proteases and collagenases. The very small cells from the myocytes and tissue debris were separated by selection for c-kit^{pos} cells. These cells can either be cloned to select the Oct 4^{pos} cells or plated at high density. The Oct 4^{pos} cells form rounded and loosely attached clones as shown in FIGS. 11 and 14. From an atrial apendice ˜3×10⁶ Lin^{neg} c-kit^{pos} cells are routinely isolated. Of these between 5 and 10% are Oct4^{pos} when analyzed after isolation by immunohistochemistry. Of these Oct4^{pos} cells >90% express the majority of the multipotency genes when expanded and cloned.

b) Small myocardial tissue explants, either obtained from necropsy specimens, surgical or catheter biopsies, were seeded. A halo of cells migrates from the explants, a small percentage of which are c-kit^{pos} cells. From these cells either by single cell cloning or by plating as indicated in “a”, it is possible to obtain clones of Oct 4^{pos} cells. Examples of such an outgrowth are shown in FIG. 11.

c) The cells of the invention can also be isolated from the side-population of cells (see FIG. 12).

The detailed procedures for these isolations, as well as the solutions used, are the same as those described for the isolation of the cells from the murine tissues.

### Example 21

### Isolation of Adult c-Kit^{pos} Oct-4^{pos} Multipotent Cells from the Human Bone Marrow

To isolate c-kit^{pos} Oct-4^{pos} cells from the human bone marrow, frozen bone marrow from a single healthy donor was purchased from Lonza (Lonza Walkerwille, Inc) (cat #2M-125D) containing ˜145×10⁶ mononucleated cells. The CD45^{pos} and 34^{pos} were removed with the appropriate Mylteni beads which yielded 35×10⁶ cells. The c-kit^{pos} cells were isolated using an anti human c-kit antibody and a Mylteni column as described for Example 1. A total of 3×10⁶ Lin^{neg} c-kit^{pos} were obtained from the whole sample. Analysis by immunohistochemistry of these cells after cytospinning them of slides showed that ˜4% of the cells were Oct4^{pos} Nanog^{pos} (FIG. 29). An aliquot of these cells was plated for cloning. At three weeks the cloning efficiency was 13% of the plated cells. Five clones were selected for expansion and further characterization. All 5 clones showed expression of all major multipotency genes as well as TERT. One of these clones was tested in vitro for its regenerative capacity (see Example 23).

### Example 22

### Human Adult c-Kit^{pos} Oct-4^{pos} Stem Cells Express all Major Pluripotency Markers

The isolated CSCs were tested for expression of various stem cell markers. To this end, RT-PCR was performed as described earlier.

The results obtained demonstrate that human Oct-4^{pos} cells express Oct-4, Nanog, Sox-2, c-myc, Klf-4, c-kit, MDR-1, BMI-1, TERT, CD 44, CD63, CD71, CD90, CD105, CD133, CD166, SSEA4, Gata-4 and Gata-6. The cells were negative for CD34 and CD45 as well as for all the blood cell lineage markers CD11b, CD 13, CD 14, CD 29, CD 31, CD 33, CD 34, CD 36, CD 38, CD 45, CD 49f, CD 62, CD 73, and CD 106 (FIG. 13).

These cells when plated one cell per well or in larger dishes at very low density are able to grow clones at a very high frequency, particularly for human primary cells. On the average, ˜16% of the plated cells form clones in a two week period. The phenotype of these clones is very stable and the expression of the multipotency genes and TERT is maintained through many generations (see FIG. 30) while the cells have a normal karyotype past 70 passages in culture (FIG. 31).

### Example 23

### Human Adult c-kit^{pos} Oct-4^{pos} Stem Cells can Differentiate into a Variety of Tissues

When grown in suitable culture conditions, the cells of the invention form pseudo-embryoid bodies. It is noteworthy that expression of c-kit and Oct-4 was lost first in the peripheral cells of the embryoid body, in accordance with the widely accepted hypothesis that cells at the perimeter differentiate first (FIG. 14). After two weeks of culture, the cells had differentiated into cardiac myocytes, smooth vascular cells and endothelial cells.

### Example 24

### Human Adult c-kit^{pos} Oct-4^{pos} Stem Cells Isolated from the Myocardium and the Bone Marrow have Strong Myocardial Regenerative Capacity in Immunodeficient Rat Hearts

In order to test the regenerative potential of the cloned c-kit^{pos} Oct-4^{pos} human cells we injected intramyocardically 5×10⁵ cells isolated from a male to each of 5 immunodeficient female rats (nu/nu) after myocardial infarction as previously described (Beltrami et al., 2003). 5 animals were injected with c-kit^{pos} Oct-4^{pos} isolated from the bone marrow and 5 animals were treated with similar cells isolated from a right atrial biopsy. The animals injected with atrial cells survived up 14 days, while only 2 of those injected with bone marrow cells did so. Four of these 5 animals showed a robust regeneration of the infarcted area which was indistinguishable whether the animal had been treated with bone marrow or with atrial cells. The regenerated area were shown to be constituted by human cells as demonstrated by their positive hybridization to human repetitive sequences which do not cross-hybridize with the rat genome. In addition, the cells in the regenerated area were positive when tested with a human specific mitochondrial probe (see FIG. 16).

### Example 25

### Human Adult Stem Cells do not Trigger a Significant Immune Response

The cells of the invention are unable to trigger a mixed lymphocyte reaction (MLR). Normally, co-culturing T cells from one individual with cells from another non-matched individual results in the proliferation of the T cells, the so-called MLR. However, when the cardiac stem cells of this invention are cultured with allogeneic T-lymphocytes, they do not stimulate the proliferation of the T-lymphocytes.

It has also been discovered that the adult cardiac stem cells actively and in a dose dependent manner reduce the immunologic response of T lymphocytes to other fully immunogenic allogeneic cells. Thus, adult cardiac stem cells of the invention need not be matched to the target cells in an MLR in order to inhibit the proliferative response of alloreactive T cells. This behaviour suggests that the adult cardiac stem cells produce immunomodulatory molecules capable of inhibiting the T lymphocyte response to allogeneic cells.

Interestingly, the molecular explanation for this “immunotolerance” of the cells is different from that reported by the mesenchymal stem cells (MSCs). As shown in FIG. 15, MSCs express the MHC-I antigens but neither MHC-II nor any of the co-stimulatory molecules, while adult human skin fibroblast express MHC-I and CD-40, which explains their low level of antigenicity.

Surprisingly, cardiac stem cells express neither MHC-I, MHC-II, CD80, CD86 nor CD40, a phenotype which should make these cells totally unrecognizable by the immune system. With the exception of adult red blood cells, these are the only normal adult cells described so far which do not express any of the molecules of the major histocompatibility locus. It has been reported that some neoplastic cells lose the expression of MHC-1 and co-stimulatory molecules and, for this reason, become invisible to the immunological surveillance. This phenotype establishes an additional difference between the cells of the invention and the adult stem cells reported to date.

The low immunogenicity of the CSCs was further demonstrated by transplanting humans CSCs to the border zone of acutely infarcted immunodeficient rats (nu/nu). The rats transplanted with human cells had a significantly better ventricular function than the controls, as determined by cardiac echocardiography. The pathology of the transplanted hearts showed reduced remodeling of the transplanted hearts and the presence of myocytes, microvessels and capillaries of human origin which were not present in the placebo transplanted hearts. The transplanted rats showed no immune response to the human cells.

In summary these data demonstrate that the adult CSCs of the invention do not provoke an immune response and also have the potential to regenerate a cardiac injury.

### Example 26

### Optimization of Culture Media for the Growth, Expansion and Maintenance of Self-Renewal Properties of the c-Kit^{pos} Oct4^{pos} Murine and Human Cells

For long-term culture of the stem cells of the invention it is a requirement that the culture medium preserve the self-renewal capabilities of the cells. Otherwise the cells progressively differentiate and after several passages the true stem cells in the culture have disappeared and the culture constitutes a mixture of precursors, progenitors and differentiated cells.

The starting “Growth Medium” for the cells of the invention is Dulbecco's MEM/Ham's F12 (DMEM/F12) modified medium containing 10% FBS, bFGF (10 ng/ml), insulin-transferrin-selenite (ITS), and EPO (2.5 U). “Differentiation medium” is medium constituted by a 1:1 ratio of DMEM/F12, bFGF (10 ng/ml), EGF (20 ng/ml), ITS, and Neural Basal Media supplemented with B27 and N2 supplements (Gibco), for the generation of cardiospheres from the myocardial derived c-kit^{pos} Oct4^{pos} cells (also referred here as embryonic bodies independently of the tissue of origin of the cells).

In order to maintain the undifferentiated state of the murine c-kit^{pos} Oct4^{pos} in vitro, four different culture media were tested.

Medium I: 10% embryonic stem cell-qualified fetal bovine serum (ES-FBS, Invitrogen); 10 ng/ml mouse basic fibroblast growth factor (FGFb, PeproTech), 20 ng/ml mouse endothelial growth factor (EGF, PeproTech), 10 ng/ml mouse leukaemia inhibitory factor (LIF, Chemicon); 6.7 ng/ml sodium selenite, 10 μg/ml insulin, 5.5 mg/ml transferring, 2 mg/ml ethanolamine (ITS, Invitrogen); 50 μg/ml gentamycin, 0.1 mg/ml streptomycin and 100 U/ml penicillin (Sigma); 250 ng/ml amphotericin B, 205 ng/ml sodium deoxycholate (Fungizone, Invitrogen) in 1:1 Dulbecco's Modified Eagle's Medium/Nutrient Mixture F-12 Ham (DMEM/F12, Sigma).

Medium II: the same than Medium I, but in this case the serum was depleted of differentiation factors and other high molecular weight proteins by treatment with DCC solution, prepared as followed: 0.45 g of dextran T500 and 4.5 g activated charcoal (Sigma) were stirred overnight at 4° C. in 1800 ml 0.01M Tris-HCl (Sigma), pH 8.0 in a tightly closed Erlenmeyer bottle. DCC solution was centrifuged at 2000 g for 20 mM in 50 ml plastic tubes, the supernatant was discarded and new DCC solution was added to the same tubes and centrifuged again, in order to obtain “double pellets”. After inactivating the FBS 30 min at 56° C., 50 ml of FBS was mixed with each double pellet and transferred to a glass bottle, incubating the mixture for 45 min at 45° C. under shaking. Afterwards, the mixture was centrifuged 20 min at 2000 g and the supernatant was mixed with a new DCC double pellet and incubated again 45 min at 45° C. in a glass bottle under shaking. After centrifuging 20 min at 2000 g, the FBS supernatant was sterilized through a 0.22 μm low protein binding filter.

Medium III: 10 ng/ml mouse basic fibroblast growth factor (FGFb, PeproTech), 10 ng/ml mouse endothelial growth factor (EGF, PeproTech), 10 ng/ml mouse leukaemia inhibitory factor (LIF, Chemicon); 0.1 mM 2-mercaptoethanol, 1 mM L-glutamate, 15 nM sodium selenite, 25 μg/ml BSA (Sigma); 0.5× Bottenstein's N-2 supplement, 0.5×B27 supplement without vitamin A (Invitrogen); 50 μg/ml gentamycin, 0.1 mg/ml streptomycin and 100 U/ml penicillin (Sigma); 250 ng/ml amphotericin B, 205 ng/ml sodium deoxycholate (Fungizone, Invitrogen) in 1:1 Neurobasal (Invitrogen) and DMEM/F 12 (Sigma) media.

Medium IV: 10% embryonic stem cell-qualified fetal bovine serum (ES-FBS), 5% horse serum (Invitrogen); 10 ng/ml mouse basic fibroblast growth factor (FGFb, PeproTech), 20 ng/ml mouse endothelial growth factor (EGF, PeproTech), 10 ng/ml mouse leukaemia inhibitory factor (LIF, Chemicon); 5 mU/ml erythropoietin, 50 μg/ml porcine gelatin, 0.2 mM L-glutathione, 50 μg/ml gentamycin, 0.1 mg/ml streptomycin and 100 U/ml penicillin (Sigma); 250 ng/ml amphotericin B, 205 ng/ml sodium deoxycholate (Fungizone, Invitrogen) in F-12K nutrient mixture with Kaighn's modification (Invitrogen), pH 7.4.

Medium IV proved to be the most effective preparation for the long term maintenance of the mouse c-kit^{pos} Oct4^{pos} cells. After 10 passages in this medium, the cloning efficiency of the cells was 63%.

To identify the best condition for the growth and maintenance of the self-renewal potential of the human c-kit^{pos} Oct4^{pos}, the following protocol was carried out: 2.5×10⁴ c-kit^{pos} Oct4^{pos} cells were plated in 60×35 mm dishes and were serum starved for 36 hrs in 0% serum growth medium. 6 dishes acted as baseline control and were supplemented with BrdU (1 ug/ml) before being fixed and stained 1 hour later. Then in serum depleted (1% ESQ-FBS) growth medium, 200 ng/ml Wnt3a (6 dishes) or 100 ng/ml HGF (n=6 dishes), or 5 ng/ml TGFβ-1 (n=6 dishes), or 10 ng/ml BMP-2 (n=6 dishes), or 10 ng/ml BMP-4 (n=6 dishes), or 10 ng/ml FGF-2 (n=6 dishes), or 100 ng/ml IGF-1 (n=6 dishes) or 5 ng/ml Wnt5a (n=6 dishes) was supplemented to the remaining 48 dishes. 6 dishes acted as controls, with no growth factors added to the medium. BrdU was added, 1 μg/ml every 6 hours. Recombinant growth factors were obtained from Peprotech and R&D Systems. Cells were fixed after 24 hours and BrdU incorporation was assessed using the BrdU detection system kit (Roche). The nuclei were counterstained with the DNA binding dye, 4,6-diamidino-2-phenylindole (DAPI, Sigma) at 1 μg/ml. Cells were evaluated using fluorescence microscopy (Nikon E1000M). 10 random fields at ×20 magnification were counted for each dish, and numbers expressed as a percentage of BrdU positive cells relative to the total number of cells counted. The results of the assay are shown in FIG. 32.

1-61. (canceled)

62. A medical device containing a composition comprising isolated adult stem cells characterized in that the cells are capable of differentiating into mesoderm, endoderm and ectoderm without recombinant manipulation, wherein the cells express: (i) one or more genes from c-kit, Nanog and Oct-4; (ii) one or more genes from SSEA1, Rex1 and Mph1 and Eed; (iii) one or more genes from MDR-I, TERT, CD133, Gata-4, Gata-6, SOX-2, klf-4, c-myc, CD90, CD166 and Bmi-1; or (iv) one or more genes from IsI-I, FoxD3, MeI-18, M33, Mph1/Rae-28, SDF1/CXCL12, CXCR4, BMP2, BPM-4, Wnt-3A, Wnt-4, and Wnt-11.

63-64. (canceled)

65. The medical device of claim 62 wherein the cells do not naturally express one or more, genes from CdI Ib, CD14, CD29, CD31, CD33, CD36, CD38, CD49f, CD62, CD73, CD105, and CD 106.

66. The medical device of claim 62 wherein the cells express c-kit at a level of between 10⁻³ and 10⁻⁶ mRNA copies per cell relative to GAPDH.

67. The medical device of claim 62 wherein the cells express Nanog at a level of between 10⁻² and 10⁻³ mRNA copies per cell relative to GAPDH.

68. The medical device of claim 62 wherein the cell express Oct-4 at a level of between 10⁻³ and 10⁻⁴ mRNA copies per cell relative to GAPDH.

69. The medical device of claim 62 wherein the cells express telomerase.

70. The medical device of claim 62 wherein the cells do not demonstrate gap junction intracellular communication (GJIC).

71. The medical device of claim 62 wherein the cells do not naturally express or express low levels of MHC I, MHC II, or co-adjuvant genes of MHC I.

72. The medical device of claim 62 wherein the cells do not trigger an immune response in the donor.

73. The medical device of claim 62 wherein the cells do not trigger an immune response when administered to an allogeneic recipient.

74. The medical device of claim 62 wherein the cells do trigger a weak immune response when administered to an allogeneic recipient.

75. The medical device of claim 62 wherein injection of the cells into a host organism does not induce production of a teratoma.

76. The medical device of claim 62 wherein the cells have the capacity to differentiate into any tissue cell-type of the body.

77. The medical device of claim 62 wherein the cells have the capacity to differentiate into cardiac tissue.

78. The medical device of claim 62 wherein the cells have the capacity to differentiate into spleen tissue, bone marrow, lung tissue, skin, intestinal tissue, liver tissue, brain tissue or skeletal muscle.

79. The medical device of claim 62 wherein the cells when injected into systemic circulation target their tissue of origin and/or to freshly damaged tissues.

80. The medical device of claim 62 wherein the cells are capable of growing in growth medium without differentiation.

81. The medical device of claim 62 wherein the cells can be passaged up to 300 times without differentiation.

82. The medical device of claim 62 wherein the cells can be passaged for up to 3 years without differentiation.

83. The medical device of claim 62 wherein the cells do not undergo detectable chromosomal rearrangements during passaging.

84. The medical device of claim 62 wherein the cellular morphology of the adult stem cells resembles a totipotent stem cell.

85. The medical device of claim 62 wherein the cellular morphology of the adult stem cells resembles a multipotent stem cell.

86. The medical device of claim 62 wherein the cells are capable of forming embryoid bodies.

87. The medical device of claim 62 wherein the cells are capable of self-renewing.

88. The medical device of claim 62 wherein the cells are clonogenic.

89. The medical device of claim 62 wherein the cells control differentiation fate of their surrounding progeny.

90. The medical device of claim 62 wherein the cells are mammalian adult stem cells.

91. The medical device of claim 62 wherein the cells are human adult stem cells.

92. The medical device of claim 62 wherein the cells are isolated from cardiac tissue.

93. The medical device of claim 62 wherein the cells are isolated from post-embryonic myocardium.

94. The medical device of claim 62 wherein the cells are isolated from post-natal cardiac tissue.

95. The medical device of claim 62 wherein the cells are isolated from adult cardiac tissue

96. The medical device of claim 62 wherein the cells are isolated from any adult tissue such as bone marrow tissue, pancreas tissue, liver tissue, skeletal muscle tissue, or central nervous tissue.

97. The medical device of claim 62 wherein the cells are in a population of cells and comprises at least 80% adult stem cells.

98. The medical device of claim 62 wherein the composition comprises isolated adult stem cells and a pharmaceutically acceptable carrier.

99. The medical device of claim 62 wherein the cells are autologous with respect to a patient receiving treatment.

100. The medical device of claim 62 wherein the cells are allogeneic but immunologically matched with respect to a patient receiving treatment.

101. The medical device of claim 62 wherein the cells are allogeneic with respect to a patient receiving treatment.

102. A method of tissue regeneration treatment or tissue repair treatment using the medical device of claim 62.

103. The method of claim 96 wherein the tissue is cardiac tissue, myocardium, central nervous tissue, skeletal muscle tissue, epithelial tissue, hepatic tissue, pancreatic tissue or pulmonary tissue.

104. The medical device of claim 62 wherein the medical device is a syringe.

105. The medical device of claim 62 wherein the medical device is a catheter.

106. The medical device of claim 62 wherein the medical device is an implant.

107. The medical device of claim 62 wherein the composition comprises a matrix.

108. The medical device of claim 62 wherein the cells are encapsulated in hollow microspheres.

109. The medical device of claim 102 wherein the microspheres comprise one or more biodegradable polymers.

110. A method for isolating adult stem cells comprising:
a) providing a suspension comprising a population of adult stem cells; and
b) selecting cells that express two or more proteins from c-kit, oct-4, and nanog.

111. A method for isolating adult stem cells comprising:
a) providing a suspension comprising a population of adult stem cells; and
b) selecting cells that express two or more proteins from c-kit, oct-4, nanog, Sox2 and Klf4.

112. A method for isolating adult stem cells comprising:
a) providing a suspension comprising a population of adult stem cells; and
b) selecting cells that express two or more proteins from c-kit, CXCR4, CD13.

113. A method for isolating adult stem cells comprising:
a) providing a suspension comprising a population of adult stem cells; and
b) selecting cells that express one or more proteins from c-kit, oct-4, and Nanog and are negative for CD45.

114. A method for isolating adult stem cells comprising:
a) providing a suspension comprising a population of adult stem cells; and
b) selecting cells that express one or more proteins from c-kit, oct-4, and Nanog and are negative for CD34.

115. The method of claim 110 wherein the cells that express c-kit or SSEA1 is selected by immuno-affinity separation or FACS analysis.

116. The method of claim 115 wherein the cells are found in side population when the cells are selected by FACS analysis.

117. An isolated naturally tripotent population of adult stem cells.
